SEQUENCE LISTING (20 sequences)

SEQ ID NO: 1 - DNA, 22 bp, Artificial sequence
  Single strand DNA oligonucleotide
gaaccagaggggagagacagag

SEQ ID NO: 2 - DNA, 22 bp, Artificial sequence
  Single strand DNA oligonucleotide
ccctcagcttgctttttaggag

SEQ ID NO: 3 - DNA, 22 bp, Artificial sequence
  Single strand DNA oligonucleotide
acagagtttattgaggtgcccc

SEQ ID NO: 4 - DNA, 20 bp, Artificial sequence
  Single strand DNA oligonucleotide
aaggtgaagtgtcccagagg

SEQ ID NO: 5 - DNA, 22 bp, Artificial sequence
  Single strand DNA oligonucleotide
tattggaacatggcctctggat

SEQ ID NO: 6 - DNA, 22 bp, Artificial sequence
  Single strand DNA oligonucleotide
ggtgctgaaggctgattacgtt

SEQ ID NO: 7 - DNA, 22 bp, Artificial sequence
  Single strand DNA oligonucleotide
agacatcgcactgactgagaac

SEQ ID NO: 8 - DNA, 22 bp, Artificial sequence
  Single strand DNA oligonucleotide
gacgggtcactatctgtgcaac

SEQ ID NO: 9 - DNA, 22 bp, Artificial sequence
  Single strand DNA oligonucleotide
gtcattgctgaaaccgagaatg

SEQ ID NO: 10 - DNA, 22 bp, Artificial sequence
  Single strand DNA oligonucleotide
gcaaagtactggatgacacgct

SEQ ID NO: 11 - DNA, 25 bp, Artificial sequence
  Single strand DNA oligonucleotide
gagaacaatgagaaccttcaggaga

SEQ ID NO: 12 - DNA, 23 bp, Artificial sequence
  Single strand DNA oligonucleotide
ttctggcgccggttacagaacca

SEQ ID NO: 13 - DNA, 20 bp, Artificial sequence
  Single strand DNA oligonucleotide
cttcaagccagaggcctacg

SEQ ID NO: 14 - DNA, 20 bp, Artificial sequence
  Single strand DNA oligonucleotide
ccgcctctgtcttcttcagc

SEQ ID NO: 15 - DNA, 22 bp, Artificial sequence
  Single strand DNA oligonucleotide
ggcagcggaagaggatgctgaa

SEQ ID NO: 16 - DNA, 25 bp, Artificial sequence
  Single strand DNA oligonucleotide
gaggcaccaagttgggcatgaacga

SEQ ID NO: 17 - DNA, 22 bp, Artificial sequence
  Single strand DNA oligonucleotide
ccctgcaccagccccaatcaga

SEQ ID NO: 18 - DNA, 21 bp, Artificial sequence
  Single strand DNA oligonucleotide
cgaagcccagcccggtcaact

SEQ ID NO: 19 - DNA, 20 bp, Artificial sequence
  Single strand DNA oligonucleotide
agccacatcgctcagacacc

SEQ ID NO: 20 - DNA, 20 bp, Artificial sequence
  Single strand DNA oligonucleotide
gtactcagcggccagcatcg

(19) United States
(12) Patent Application Publication
(10) Pub. No.: US 2012/0164117 A1
(43) Pub. Date: Jun. 28, 2012

(54) METHODS OF GENERATING HUMAN CARDIAC CELLS AND TISSUES AND USES THEREOF

(75) Inventors: LIOR GEPSTEIN, HAIFA (IL); IZHAK KEHAT, HAIFA (IL); JOSEPH ITSKOVITZ-ELDOR, HAIFA (IL); MICHAL AMIT, MISGAV (IL)

(73) Assignee: TECHNION RESEARCH & DEVELOPMENT FOUNDATION LTD., HAIFA (IL)

(21) Appl. No.: 13/417,505

(22) Filed: Mar. 12, 2012

Related U.S. Application Data
(63) Continuation of application No. 12/701,648, filed on Feb. 8, 2010.
Application No. 12/701,648 is a division of application No. 10/759,734, filed on Jan. 20, 2004, now abandoned.
Application No. 10/759,734 is a continuation-in-part of application No. PCT/IL02/00606, filed on Jul. 21, 2002.
(60) Provisional application No. 60/306,462, filed on Jul. 20, 2001.

(51) Int. Cl. A61K 35/34; G01N 33/567; A61P 9/00; C12Q 1/68

(52) U.S. Cl. 424/93.7; 435/6.12; 435/7.21

(57) ABSTRACT

A method of generating cells predominantly displaying at least one characteristic associated with a cardiac phenotype is disclosed. The method comprises (a) partially dispersing a confluent cultured population of human stem cells, thereby generating a cell population including cell aggregates; (b) subjecting the cell aggregates to culturing conditions suitable for generating embryoid bodies; (c) subjecting the embryoid bodies to culturing conditions suitable for inducing cardiac lineage differentiation in at least a portion of the cells of the embryoid bodies, the culturing conditions suitable for inducing cardiac lineage differentiation including adherence of the embryoid bodies to a surface, and culture, medium supplemented with serum, thereby generating cells predominantly displaying at least one characteristic associated with a cardiac phenotype.

### RELATED APPLICATIONS

This application is a continuation of U.S. patent application Ser. No. 12/701,648 filed on Feb. 8, 2010, which is a divisional of U.S. patent application Ser. No. 10/759,734 filed on Jan. 20, 2004, which is a continuation-in-part (CIP) of PCT Patent Application No. PCT/IL02/00606 filed on Jul. 21, 2002, which claims the benefit of priority of U.S. Provisional Patent Application No. 60/306,462 filed on Jul. 20, 2001. The contents of all of the above applications are incorporated by reference as if fully set forth herein.

### FIELD AND BACKGROUND OF THE INVENTION

The present invention relates to methods of generating cardiac cells and tissue by in-vitro culture of differentiable cells, to methods of using such cardiac cells and tissues to repair dysfunctional human cardiac tissues and to test the effect of treatments on human cardiac cells and tissues, and to characterize biological states or processes of cardiac cells and tissues. More particularly, the present invention relates to methods of generating highly differentiated, highly functional, proliferating cardiac cells and tissues by in-vitro culture of human embryonic stem cells, and to methods of using such cardiac cells and tissue to repair human cardiac tissue, to test the therapeutic effect and toxicity of pharmacological and electrical treatments on human cardiac cells and tissues, and to model the development and physiology of cardiac cells and tissues in-vitro.

Heart disease is the predominant cause of disability and death in all industrialized nations, and, in addition, the incidence of heart failure is increasing in the United States, with more than half a million Americans dying of this disease yearly (Braunwald E., 1997. N Eng J Med. 337:1360; Eriksson H., 1995. J Inter Med. 237:135). In addition, in the United States, cardiac disease accounts for about 335 deaths per 100,000 individuals (approximately 40% of the total mortality) overshadowing cancer, which follows with 183 deaths per 100,000 individuals. Four categories of heart disease account for about 85-90% of all cardiac-related deaths. These categories are ischemic heart disease, hypertensive heart disease and pulmonary hypertensive heart disease, valvular disease, and congenital heart disease. Ischemic heart disease alone, notably myocardial infarction, accounts for about 60-75% of all deaths caused by heart disease. Despite considerable advances in the diagnosis and treatment of heart disease, cardiac dysfunction following myocardial infarction remains a major cardiovascular disorder that is increasing in incidence, prevalence, and overall mortality. Myocardial infarction is a life-threatening event responsible for cardiac sudden death or heart failure involving blockage of cardiac blood vessels, and concomitant death and damage of cardiac tissues induced by oxygen deprivation. Following acute myocardial infarction, dead and damaged cardiac muscle cells (cardiomyocytes) are gradually replaced by fibroid nonfunctional tissue, and in many cases ventricular remodeling results in wall thinning and loss of regional contractile function. One of the factors that renders ischemic heart disease, such as myocardial infarction, so devastating is the extremely low capacity of healthy adult cardiomyocytes to divide, and thus repopulate areas of ischemic heart damage. Thus, cardiac cell loss as a result of injury or diseases such as myocardial infarction is essentially irreversible. When occurring at critical sites in the conduction system of the heart, such cell loss or injury may lead to inefficient rhythm initiation or impulse conduction. Consequentially, these processes may result in abnormally low heart rate (bradyarrhythmias) requiring the implantation of a permanent pacemaker.

Human to human heart transplants have become the most effective form of therapy for severe heart damage. Heart transplantation, however, suffers from numerous drawbacks. For example, this form of therapy is severely limited by the scarcity of suitable donor organs, and, in addition, the expense of heart transplantation prohibits its widespread application. Another unsolved problem is graft rejection. Foreign hearts are poorly tolerated by the recipient and are rapidly destroyed by the immune system in the absence of immunosuppressive drugs, such as cyclosporin, the immunosuppressant of choice. However, drugs such as cyclosporin severely impair immune responses such as those against bacterial and viral infections, thereby placing the transplant recipient at risk of infection. Yet further complications caused by cyclosporin include hypertension, renal dysfunction, rapidly progressive coronary atherosclerosis, and immunosuppressant-related cancers. Thus, alternatives to heart transplantation for treating cardiac disorders are urgently required.

One approach for overcoming deficits of cardiac transplantation, of treating cardiac disease has suggested transplantation of adult cardiomyocytes into affected cardiac tissues since, for example, functional engraftment of such cells or tissues within dead or dysfunctional cardiac tissues could restore impaired cardiac function. However, such approaches are hampered by the inability of adult cardiomyocytes to proliferate, and hence to efficiently colonize and regenerate, dead or damaged cardiac tissue, or to remodel deformed cardiac tissue.

The extremely low capacity of adult cardiomyocytes to proliferate further represents an obstacle to approaches attempting to utilize in-vitro culture thereof to generate cells and tissues suitable for testing the therapeutic effect and toxicity of treatments, such as pharmacological and electrical treatments, on cardiomyocytic cells and tissues, and to model the development and physiology of such cells and tissues.

Several prior art approaches have attempted to use in-vitro culture of differentiable cells (cells having a capacity to differentiate) to generate cardiac cells or tissue suitable for treating human cardiac disorders, and for testing the effects of treatments, such as pharmacological and electrical treatments, on human cardiac tissues.

One approach has utilized delivery of mouse embryonic/fetal cardiomyocytes into syngeneic host myocardium in an attempt to demonstrate the engraftment capacity of such developing cells (Soonpaa, M. H. et al., 1994. Science 264:98).

Yet another approach has utilized transplantation of rat fetal myocardial tissue into infarcted rat myocardium in an attempt to repair such infarcted tissues (Leor, J. et al., 1996. Circulation 94(suppl. II):II-332-II-336).

Still another approach has used culturing of mouse embryonic stem cells in attempts to generate cardiomyocytic cells and tissue (Igelmund et al., 1999. Flugers Arch. 437:669; Maltsev V. A. et al., 1994. Circ. Res. 75:233; Metzger J M. et al., 1995. Circ Res. 76:710; Sánchez A. et al., 1991. J Biol Chem. 266:22419).

A further approach has employed genetic transformation of murine embryonic stem cells with a fusion gene consisting of the alpha-cardiac myosin heavy chain promoter and a cDNA encoding aminoglycoside phosphotransferase to generate cardiomyocytic cells (Klug, M. G. et al., 1996. J. Clin. Invest. 98:216).

Still a further approach has utilized suspension culture of cystic human embryoid bodies in an attempt to generate cardiomyocytic cells and tissues (Itskovitz-Eldor et al., 2000. Mol. Med. 6:88-95).

However, all of the aforementioned approaches are unsatisfactory for providing cells and tissues suitable for human application; approaches utilizing genetic transformation entail safety risks, pose ethical concerns, and are labor intensive, administratively difficult to implement, and unproven in human systems; and approaches utilizing suspension culture of cystic human embryoid bodies are inefficient, have not demonstrated a satisfactory range of cardiac specific structure and function, have not provided isolated human cardiac cells and tissues, have not demonstrated long term cardiac functionality in-vitro, and have not demonstrably provided cells and tissues capable of conferring cardiac function when engrafted in-vivo.

Thus, all prior art approaches have failed to provide an adequate solution for using in-vitro culture of differentiable cells to generate cardiac cells and tissues suitable for treating human cardiac disorders, for testing the therapeutic and toxic effects of treatments, such as pharmacological and electrical treatments, on human cardiac cells and tissues, and for modeling processes such as development and physiology of human cardiac cells and tissues.

There is thus a widely recognized need for, and it would be highly advantageous to have, a method of using in-vitro culture of differentiable cells to generate cardiac cells and tissues devoid of the above limitation.

### SUMMARY OF THE INVENTION

According to one aspect of the present invention there is provided a method of generating cells predominantly displaying at least one characteristic associated with a cardiac phenotype, the method comprising: (a) partially dispersing a confluent cultured population of human stem cells, thereby generating a cell population including cell aggregates; (b) subjecting the cell aggregates to culturing conditions suitable for generating embryoid bodies; and (c) subjecting the embryoid bodies to culturing conditions suitable for inducing cardiac lineage differentiation in at least a portion of the cells of the embryoid bodies thereby generating cells predominantly displaying at least one characteristic associated with the cardiac phenotype.

According to further features in preferred embodiments of the invention described below, the method of generating cells predominantly displaying at least one characteristic associated with a cardiac phenotype further comprises isolating the cell aggregates from the cell population prior to step (b).

According to still further features in the described preferred embodiments, the method of generating cells predominantly displaying at least one characteristic associated with a cardiac phenotype further comprises isolating the embryoid bodies prior to step (c).

According to still further features in the described preferred embodiments, the method of generating cells predominantly displaying at least one characteristic associated with a cardiac phenotype further comprises screening and optionally isolating cells predominantly displaying at least one characteristic associated with a cardiac phenotype, the screening effected by at least one method selected from the group consisting of detection of mechanical contraction, detection of a cardiac specific structure, detection of a cardiac specific protein, detection of a cardiac specific RNA, detection of cardiac specific electrical activity, and detection of cardiac specific changes in the intracellular concentration of a physiological ion.

According to still further features in the described preferred embodiments, the method of generating cells predominantly displaying at least one characteristic associated with a cardiac phenotype further comprises screening and optionally isolating cells substantially displaying proliferation.

According to another aspect of the present invention there is provided a method of generating tissue predominantly displaying at least one characteristic associated with a cardiac phenotype, the method comprising: (a) partially dispersing a confluent cultured population of human stem cells, thereby generating a cell population including cell aggregates; (b) subjecting the cell aggregates to culturing conditions suitable for generating embryoid bodies; and (c) subjecting the embryoid bodies to culturing conditions suitable for inducing cardiac lineage differentiation in at least a portion of the cells of the embryoid bodies thereby generating tissue predominantly displaying at least one characteristic associated with the cardiac phenotype.

According to further features in preferred embodiments of the invention described below, the method of generating tissue predominantly displaying at least one characteristic associated with a cardiac phenotype further comprises isolating the cell aggregates from the cell population prior to step (b).

According to still further features in the described preferred embodiments, the method of generating tissue predominantly displaying at least one characteristic associated with a cardiac phenotype further comprises isolating the embryoid bodies prior to step (c).

According to still further features in the described preferred embodiments, the method of generating tissue predominantly displaying at least one characteristic associated with a cardiac phenotype further comprises screening and optionally isolating tissue predominantly displaying at least one characteristic associated with a cardiac phenotype, the screening effected by at least one method selected from the group consisting of detection of mechanical contraction, detection of a cardiac specific structure, detection of a cardiac specific protein, detection of a cardiac specific RNA, detection of cardiac specific electrical activity, and detection of cardiac specific changes in the intracellular concentration of a physiological ion.

According to still further features in the described preferred embodiments, the method of generating tissue predominantly displaying at least one characteristic associated with a cardiac phenotype further comprises screening and optionally isolating tissue substantially displaying proliferation.

According to still another aspect of the present invention there is provided a method of characterizing a biological state or a biological process of cardiac cells or cardiac tissue, the method comprising: (a) partially dispersing a confluent cultured population of human stem cells, thereby generating a cell population including cell aggregates; (b) subjecting the cell aggregates to culturing conditions suitable for generating embryoid bodies; (c) subjecting the embryoid bodies to culturing conditions suitable for inducing cardiac lineage differentiation in at least a portion of the cells of the embryoid bodies thereby generating the cells predominantly displaying at least one characteristic associated with a cardiac phenotype, or the tissue predominantly displaying at least one characteristic associated with a cardiac phenotype; and (d) obtaining data characterizing the biological state or the biological process in the cells predominantly displaying at least one characteristic associated with a cardiac phenotype, or the tissue predominantly displaying at least one characteristic associated with a cardiac phenotype.

According to further features in preferred embodiments of the invention described below, the method of characterizing a biological state or a biological process further comprises isolating the cell aggregates from the cell population prior to step (b).

According to still further features in the described preferred embodiments, the method of characterizing a biological state or a biological process further comprises isolating the embryoid bodies prior to step (c).

According to still further features in the described preferred embodiments, the method of characterizing a biological state or a biological process further comprises screening and optionally isolating cells predominantly displaying at least one characteristic associated with a cardiac phenotype, or tissue predominantly displaying at least one characteristic associated with a cardiac phenotype, the screening effected by at least one method selected from the group consisting of detection of mechanical contraction, detection of a cardiac specific structure, detection of a cardiac specific protein, detection of a cardiac specific RNA, detection of cardiac specific electrical activity, and detection of cardiac specific changes in the intracellular concentration of a physiological ion.

According to still further features in the described preferred embodiments, the method of characterizing a biological state or a biological process further comprises screening and optionally isolating cells substantially displaying proliferation or tissue substantially displaying proliferation.

According to still further features in the described preferred embodiments, the method of characterizing a biological state or a biological process further comprises inducing the biological state or the biological process in the cells predominantly displaying at least one characteristic associated with a cardiac phenotype, or the tissue predominantly displaying at least one characteristic associated with a cardiac phenotype.

According to still further features in the described preferred embodiments, the method of characterizing a biological state or a biological process further comprises co-culturing the cells predominantly displaying at least one characteristic associated with a cardiac phenotype, or the tissue predominantly displaying at least one characteristic associated with a cardiac phenotype with primary cardiac cells or primary cardiac tissue prior to step (d).

According to still further features in the described preferred embodiments, the method of characterizing a biological state or a biological process further comprises transplanting the cells predominantly displaying at least one characteristic associated with a cardiac phenotype, or the tissue predominantly displaying at least one characteristic associated with a cardiac phenotype into cardiac tissue of a recipient prior to step (d).

According to still further features in the described preferred embodiments, the biological state or the biological process is selected from the group consisting of cardiac specific mechanical contraction, a cardiac specific structure, expression of a cardiac specific RNA, expression of a cardiac specific protein, cardiac specific changes in the intracellular concentration of a physiological ion, cardiac specific electrical activity, and cardiomyogenesis.

According to still further features in the described preferred embodiments, the biological state or the biological process is cardiac specific electrical activity and obtaining data characterizing the biological state or the biological process is effected using a multielectrode array.

According to yet another aspect of the present invention there is provided a method of qualifying the effect of a treatment on a biological state or a biological process of cardiac cells or tissue, the method comprising: (a) partially dispersing a confluent cultured population of human stem cells, thereby generating a cell population including cell aggregates; (b) subjecting the cell aggregates to culturing conditions suitable for generating embryoid bodies; (c) subjecting the embryoid bodies to culturing conditions suitable for inducing cardiac lineage differentiation in at least a portion of the cells of the embryoid bodies thereby generating cells predominantly displaying at least one characteristic associated with a cardiac phenotype, or the tissue predominantly displaying at least one characteristic associated with a cardiac phenotype; (d) subjecting the cells predominantly displaying at least one characteristic associated with a cardiac phenotype, or the tissue predominantly displaying at least one characteristic associated with a cardiac phenotype to the treatment; and (e) monitoring the biological state or the biological process in the cells predominantly displaying at least one characteristic associated with a cardiac phenotype, or the tissue predominantly displaying at least one characteristic associated with a cardiac phenotype, thereby qualifying the effect of the treatment on the biological state or the biological process.

According to still further features in the described preferred embodiments, the treatment is effected by subjecting the cells predominantly displaying at least one characteristic associated with a cardiac phenotype, or the tissue predominantly displaying at least one characteristic associated with a cardiac phenotype to an exposure to a compound or to an electrical treatment.

According to still further features in the described preferred embodiments, the method of qualifying the effect of a treatment on a biological state or a biological process further comprises isolating the cell aggregates from the cell population prior to step (b).

According to still further features in the described preferred embodiments, the method of qualifying the effect of a treatment on a biological state or a biological process further comprises isolating the embryoid bodies prior to step (c).

According to still further features in the described preferred embodiments, the method of qualifying the effect of a treatment on a biological state or a biological process further comprises screening and optionally isolating cells predominantly displaying at least one characteristic associated with a cardiac phenotype, or tissue predominantly displaying at least one characteristic associated with a cardiac phenotype, the screening effected by at least one method selected from the group consisting of detection of mechanical contraction, detection of a cardiac specific structure, detection of a cardiac specific protein, detection of a cardiac specific RNA, detection of cardiac specific electrical activity, and detection of cardiac specific changes in the intracellular concentration of a physiological ion.

According to still further features in the described preferred embodiments, the method of qualifying the effect of a treatment on a biological state or a biological process further comprises screening and optionally isolating cells substantially displaying proliferation or tissue substantially displaying proliferation.

According to still further features in the described preferred embodiments, the method of qualifying the effect of a treatment on a biological state or a biological process further comprises inducing the biological state or the biological process in the cells predominantly displaying at least one characteristic associated with a cardiac phenotype, or the tissue predominantly displaying at least one characteristic associated with a cardiac phenotype.

According to still further features in the described preferred embodiments, the method of qualifying the effect of a treatment on a biological state or a biological process further comprises co-culturing the cells predominantly displaying at least one characteristic associated with a cardiac phenotype, or the tissue predominantly displaying at least one characteristic associated with a cardiac phenotype with primary cardiac cells or primary cardiac tissue following step (c).

According to still further features in the described preferred embodiments, the method of qualifying the effect of a treatment on a biological state or a biological process further comprises transplanting the cells predominantly displaying at least one characteristic associated with a cardiac phenotype, or the tissue predominantly displaying at least one characteristic associated with a cardiac phenotype into cardiac tissue of a recipient following step (c).

According to still further features in the described preferred embodiments, the inducing the biological state or the biological process is effected by treating the cells predominantly displaying at least one characteristic associated with a cardiac phenotype, or the tissue predominantly displaying at least one characteristic associated with a cardiac phenotype with a treatment selected from the group consisting of a treatment with a drug, a treatment with a physiological ion, and an electrical treatment.

According to still further features in the described preferred embodiments, the drug is selected from the group consisting of 1-heptanol, isoproterenol, carbamylcholine, forskolin, IBMX, atropine, tetrodotoxin, and diltiazem hydrochloride.

According to still further features in the described preferred embodiments, the physiological ion is selected from the group consisting of a potassium ion, a sodium ion, and a calcium ion.

According to still further features in the described preferred embodiments, the recipient is a swine.

According to still further features in the described preferred embodiments, the propagative electrical activity is characterized by slow conduction.

According to still further features in the described preferred embodiments, the biological state or the biological process is cardiac specific electrical activity and monitoring the biological state or the biological process is effected using a multielectrode array.

According to still further features in the described preferred embodiments, the multielectrode array comprises electrodes positioned 100 μm or less apart.

According to still further features in the described preferred embodiments, the multielectrode array comprises at least 60 electrodes.

According to still further features in the described preferred embodiments, the multielectrode array measures electrical activity with a frequency of 10 kHz or higher.

According to an additional aspect of the present invention there is provided a method of repairing cardiac tissue in a subject, the method comprising: (a) partially dispersing a confluent cultured population of human stem cells, thereby generating a cell population including cell aggregates; (b) subjecting the cell aggregates to culturing conditions suitable for generating embryoid bodies; (c) subjecting the embryoid bodies to culturing conditions suitable for inducing cardiac lineage differentiation in at least a portion of the cells of the embryoid bodies thereby generating cells predominantly displaying at least one characteristic associated with a cardiac phenotype, or tissue predominantly displaying at least one characteristic associated with a cardiac phenotype; and (d) administering a therapeutically effective dose of the cells predominantly displaying at least one characteristic associated with a cardiac phenotype, and/or the tissue predominantly displaying at least one characteristic associated with a cardiac phenotype to the heart of the subject, thereby repairing cardiac tissue in the subject.

According to further features in preferred embodiments of the invention described below, the method of repairing cardiac tissue further comprises isolating the cell aggregates from the cell population prior to step (b).

According to still further features in the described preferred embodiments, the method of repairing cardiac tissue further comprises isolating the embryoid bodies prior to step (c).

According to still further features in the described preferred embodiments, the method of repairing cardiac tissue further comprises screening and optionally isolating cells predominantly displaying at least one characteristic associated with a cardiac phenotype or tissue predominantly displaying at least one characteristic associated with a cardiac phenotype, the screening effected by at least one method selected from the group consisting of detection of mechanical contraction, detection of a cardiac specific structure, detection of a cardiac specific protein, detection of a cardiac specific RNA, detection of cardiac specific electrical activity, and detection of cardiac specific changes in the intracellular concentration of a physiological ion.

According to still further features in the described preferred embodiments, the detection of cardiac specific electrical activity is effected using a microelectrode array.

According to still further features in the described preferred embodiments, the multielectrode array comprises electrodes positioned 100 μm or less apart.

According to still further features in the described preferred embodiments, the multielectrode array comprises at least 60 electrodes.

According to still further features in the described preferred embodiments, the multielectrode array is configured to obtain data characterizing the cardiac specific electrical activity with a frequency greater than a range selected from 1-25 kHz.

According to still further features in the described preferred embodiments, the method of repairing cardiac tissue further comprises screening and optionally isolating cells substantially displaying proliferation or tissue substantially displaying proliferation.

According to still further features in the described preferred embodiments, the method of repairing cardiac tissue further comprises treating the subject with an immunosuppressive regimen, thereby promoting engraftment of the cells predominantly displaying at least one characteristic associated with a cardiac phenotype, or the tissue predominantly displaying at least one characteristic associated with a cardiac phenotype in the subject.

According to still further features in the described preferred embodiments, the method of repairing cardiac tissue further comprises inactivating or removing pathogenic cardiac cells or pathogenic cardiac tissue in the subject.

According to still further features in the described preferred embodiments, the administering is effected by injection of the cells predominantly displaying at least one characteristic associated with a cardiac phenotype, or the tissue predominantly displaying at least one characteristic associated with a cardiac phenotype into the heart of the subject.

According still further features in the described preferred embodiments, the culturing conditions suitable for inducing cardiac lineage differentiation include adherence of the embryoid bodies to a surface.

According to still further features in the described preferred embodiments, the human stem cells are embryonic stem cells.

According to still further features in the described preferred embodiments, the human stem cells are syngeneic with the subject.

According to still further features in the described preferred embodiments, the partially dispersing a confluent cultured population of human stem cells is effected via a non-trypsin based method.

According to still further features in the described preferred embodiments, the partially dispersing a confluent cultured population of human stem cells is effected via treatment with collagenase.

According to still further features in the described preferred embodiments, the culturing in step (b) is effected for a time period selected from the range of 1 to 20 days.

According to still further features in the described preferred embodiments, the culturing conditions in step (b) include inhibiting adherence of the cell aggregates to a surface.

According to still further features in the described preferred embodiments, the culturing conditions in step (b) include culture medium supplemented with serum.

According to still further features in the described preferred embodiments, the culturing in step (c) is effected for a time period selected from the range of 1-60 days.

According to still further features in the described preferred embodiments, the culturing in step (c) is effected in the presence of dimethyl sulfoxide.

According to still further features in the described preferred embodiments, the culturing conditions in step (c) include exposing the embryoid bodies to a surface coated with gelatin.

According to still further features in the described preferred embodiments, the culturing conditions suitable for inducing cardiac lineage differentiation further include culture medium supplemented with serum.

According to yet an additional aspect of the present invention there is provided an in-vitro culture of isolated human cells which will display substantial proliferation for at least as long as a time period selected from the range of 1-35 days, and which will predominantly display at least one characteristic associated with a cardiac phenotype for at least as long as a time period selected from the range of 1-60 days.

According to still an additional aspect of the present invention there is provided an in-vitro culture of an isolated human tissue which will display substantial proliferation for at least as long as a time period selected from the range of 1-35 days, and which will predominantly display at least one characteristic associated with a cardiac phenotype for at least as long as a time period selected from the range of 1-60 days.

According to still further features in the described preferred embodiments, the at least one characteristic associated with a cardiac phenotype is selected from the group consisting of cardiac specific mechanical contraction, a cardiac specific structure, expression of a cardiac specific RNA, expression of a cardiac specific protein, cardiac specific changes in the intracellular concentration of a physiological ion, and cardiac specific electrical activity.

According to still further features in the described preferred embodiments, the cardiac specific mechanical contraction is selected from the group consisting of spontaneous mechanical contraction, rhythmic mechanical contraction, synchronous mechanical contraction, and propagative mechanical contraction.

According to still further features in the described preferred embodiments, the cardiac specific structure is selected from the group consisting of a sarcomere, a Z-band, an intercalated disc, a gap junction, a desmosome, a fibrillar bundle, a fibrillar bundle striation, and a myocytic syncytium.

According to still further features in the described preferred embodiments, the cardiac specific RNA encodes a protein selected from the group consisting of cardiac α-myosin heavy chain, cardiac β-myosin heavy chain, α-actinin, cardiac troponin I, cardiac troponin T, GATA-4, Nkx2.5, MLC-2A, MLC-2V, atrial myosin light chain, ventricular myosin light chain, and connexin-43.

According to still further features in the described preferred embodiments, the cardiac specific protein is selected from the group consisting of cardiac α-myosin heavy chain, cardiac β-myosin heavy chain, atrial natriuretic peptide, cardiac troponin I, desmin and connexin-43.

According to still further features in the described preferred embodiments, the cardiac specific electrical activity is selected from the group consisting of spontaneous electrical activity, rhythmic electrical activity, synchronized electrical activity, and propagative electrical activity.

According to further features in the described preferred embodiments, the isolated human cells are cultured in contact with a multielectrode array configured for monitoring the cardiac specific electrical activity.

According to further features in the described preferred embodiments, the isolated human tissue is cultured in contact with a multielectrode array configured for monitoring the cardiac specific electrical activity.

According to still further features in the described preferred embodiments, the subject is a human or a nonhuman mammal.

According to still further features in described preferred embodiments, the subject has a cardiac disorder characterized by cardiac arrhythmia, and the administering is effected by intra-myocardial injection of the cells predominantly displaying at least one characteristic associated with a cardiac phenotype, or tissue predominantly displaying at least one characteristic associated with a cardiac phenotype, thereby treating the disorder characterized by cardiac arrhythmia.

According to still further features in described preferred embodiments, the subject has a cardiac disorder characterized by abnormal generation of the electrical impulse or impaired conduction, and whereas the administering is effected by intra-myocardial injection of the cells predominantly displaying at least one characteristic associated with a cardiac phenotype, or the tissue predominantly displaying at least one characteristic associated with a cardiac phenotype, thereby treating the disorder characterized by impaired cardiac conducting tissue.

According to still further features in described preferred embodiments, the subject has a cardiac disorder characterized by myocardial ischemia, and the administering is effected by intra-myocardial injection of the cells predominantly displaying at least one characteristic associated with a cardiac phenotype, or tissue predominantly displaying at least one characteristic associated with a cardiac phenotype, thereby treating the disorder characterized by myocardial ischemia.

The present invention successfully addresses the shortcomings of the presently known configurations by providing a method of using in-vitro culture of human differentiable cells to generate highly differentiated, highly functional cardiac cells and tissues.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1a is a schematic diagram depicting the three stages of differentiation of human embryonic stem cells into cells and tissues displaying cardiomyocytic characteristics. Initially, the embryonic stem cell colonies are grown on top of the mouse embryonic feeder (MEF) feeder layer (left). To induce differentiation, cells are transferred to suspension, where they aggregate to form embryoid bodies (middle). Following 10 days in suspension, embryoid bodies are plated on gelatin-coated culture dishes, where they are examined for the appearance of spontaneous contractions (right).

FIGS. 1b-d are photomicrographs depicting cells during the three stages of differentiation of human embryonic stem cells into cells displaying cardiomyocytic characteristics: embryonic stem cell colony (FIG. 1b), embryoid bodies in suspension (FIG. 1c), and a contracting area in the outgrowth on an embryoid body (FIG. 1d).

FIG. 2 is a data plot depicting cumulative percentage of embryoid bodies containing spontaneously contracting areas as a function of the number of days postplating of the embryoid body. Embryoid bodies were found to display plateau levels of contraction for at least 60 days, the longest time period studied (not shown in Figure).

FIGS. 3a-e are transmission electron micrographs depicting cardiomyocyte specific ultrastructural characteristics of embryonic stem cell-derived cardiomyocytic cells. FIG. 3a depicts a section of a beating embryoid body 10 days postplating. Relatively unorganized myofibrillar bundles can be seen in some myocytes. FIG. 3b depicts a cell 27 days postplating displaying a more mature sarcomeric organization. FIG. 3c depicts different cells from the same embryoid body as in FIG. 3b demonstrating more organized sarcomeres and Z-bands (arrow). FIGS. 3d and 3e are high-power electron micrographs showing the presence of a gap junction (arrow) and desmosomes (arrow), respectively.

FIGS. 4a-f are fluorescence photomicrographs depicting expression of cardiac muscle specific, but not skeletal muscle specific proteins in embryonic stem cell-derived cardiomyocytic cells. FIG. 4a depicts immunostaining of dispersed cells from a beating embryoid body (day 16 postplating) with anti cardiac α/β-myosin heavy chain mAbs. Several cells stained positively (×40 magnification). FIG. 4b depicts immunostaining with anti cardiac α/β-myosin heavy chain mAbs of dispersed cells from a beating embryoid body of a cardiomyocyte (day 16 postplating) at a more developed stage (×63 magnification). Note the appearance of early striation pattern (arrow). FIG. 4c depicts positive staining with anti sarcomeric α-actinin mAbs (day 17 postplating; ×63 magnification). FIG. 4d depicts positive staining with cTnI mAbs (day 30 postplating; ×63 magnification). FIG. 4e depicts positive staining with anti desmin mAbs (day 18 postplating; ×63 magnification). FIG. 4f depicts positive staining with anti atrial natriuretic peptide antibody (day 16 postplating ×63 magnification).

FIG. 5 is a series of fluorescence photographs depicting expression of cardiac specific genes in the contracting embryoid bodies. RNA samples from undifferentiated embryonic stem cells (ES) and contracting embryoid bodies (C-EBs) were analyzed by RT-PCR for the expression of cardiac-specific markers: cardiac troponin I (cTnI), cardiac troponin T (cTnT), GATA-4, human atrial natriuretic peptide (hANP), MLC-2A, MLC-2V, Nkx2.5, and α-myosin heavy chain (α-MHC). Octamer-binding protein 4 (Oct-4) is an embryonic stem cell marker. GAPDH served as internal standard.—RT indicates no cDNA.

FIGS. 6a-b are data plots depicting a typical calcium transient (FIG. 6a) and a continuous recording of calcium transients (FIG. 6b) in cultured human embryonic stem cell-derived cardiomyocytic cells as determined by fura-2 fluorescence. Tp—time to peak, T_{T}—total transient time, and T_{1/2}—time to half-peak relaxation, ms—milliseconds.

FIG. 6c is a data plot depicting typical extracellular electrophysiological recordings from different areas of the embryoid body. Note the presence of a sharp and slow component. ms—milliseconds.

FIG. 7 is a photograph depicting a microelectrode array (MEA) plate, which consists of 60 electrodes spaced 100 μm apart.

FIGS. 8a-d are fluorescence photomicrographs depicting immunostaining of contracting areas within the EBs. FIG. 8a—anti-cTnI antibodies (red) and ToPro3 (nuclear staining, blue); FIG. 8b—double staining using anti-cTnI antibodies (red) and anti-Cx45 antibodies (green); FIG. 8c—confocal immunostaining using anti Cx43 antibodies; FIG. 8d—confocal immunostaining using anti Cx45 antibodies. FIGS. 9a-d depict multielectrode electrophysiological analysis of a unifocal contracting embryoid body. FIG. 9a is a photomicrograph depicting a contracting embryoid body plated on a microelectrode array plate. FIG. 9b is a set of data plots depicting typical extracellular recordings from all 60 electrodes of the microelectrode array. FIG. 9c is a data plot depicting a high-resolution activation map generated from LATs measured at each electrode. Note that, in this example, activation propagates from the lower left side of the microelectrode array (earliest site—red) towards the right upper area (latest activation site—blue). A conduction velocity vectorial map, represented by arrows whose sizes correlate with the local conduction velocity, is superimposed on the activation map. FIG. 9d is a histogram depicting the resulting activation time histogram of the activation map depicting in FIG. 9c, characterized by a single cluster of activation times, which is continuous from early to late LATs, typical of an embryoid body with a broad conducting area.

FIGS. 10a-c depict multielectrode electrophysiological analysis of an embryoid body with two contracting areas connected through a narrow conducting zone. FIG. 10a is fluorescence photomicrograph depicting immunostaining of the embryoid body with anti cTnI antibody (green). Note the narrow connecting region between the two contracting areas located between the two gray markers. FIG. 10b is an activation map of the contracting areas showing the presence of early activated sites (red and yellow) in the top portion of the microelectrode array and late activated sites (blue and dark blue) in the lower part, with slow conduction between these two zones. Black areas indicate electrodes in which no electrical activity was recorded. The gray markers allow comparison between this activation map and the micrograph in FIG. 10a since they mark the same electrodes in both figures. Note the spatial correlation between the narrow connecting region and the area of slow conduction. FIG. 10c is a histogram depicting early and late activation times clusters with a paucity of intermediate values.

FIGS. 11a-d depict activation maps of the same embryoid body depicting the slowing of conduction induced by gradual elevation of extracellular potassium concentration. Note the significant slowing of conduction that was manifested by an increase in total activation time from a baseline value of 25 milliseconds (5.3 mM, FIG. 11a) to values of 40, 70, and 120 milliseconds at extracellular potassium concentrations of 10, 15, and 20 mM (FIGS. 11b-d, respectively).

FIGS. 12a-b depict activation maps of the same embryoid body during baseline (FIG. 12a) and following application of 10 μM TTX (FIG. 12b). Note the increase in total microelectrode array activation time from a baseline value of 15 milliseconds to a value of 35 milliseconds in this example.

FIGS. 13a-b depict activation maps of the same embryoid body during baseline (FIG. 13a) and following application of 0.3 mM of 1-heptanol (FIG. 13b). Reduction of cell-to-cell coupling by 1-heptanol resulted in slowing of conduction, as manifested by an increase of total activation time from a baseline value of 15 to a value of 32 milliseconds.

FIGS. 14a-b are photomicrographs of a structurally inhomogeneous embryoid body and its activation map, respectively, depicting the effect of structural inhomogeneities (FIG. 14a) on microconduction (FIG. 14b). Note that the presence of noncontracting tissue (identified by the set of 3 parallel arrows) resulted in alteration of the propagation pathway. The resulting activation wavefront curvature (identified by the angled arrow) was associated with significant slowing of conduction, as can be appreciated by the prolonged total microelectrode array activation time.

FIGS. 15a-b depict a microelectrode array generated high-resolution activation map (FIG. 15a) and positive chronotropic responses following addition of 10 μM isoproterenol (FIG. 15b, indicated by arrow) of spontaneously contracting cultured human embryonic stem cell-derived cardiomyocytic cells used to generate hybrid rat ventricular myocyte-human embryonic stem cell-derived cardiomyocyte co-cultures. FIG. 16a depicts a phase-contrast photomicrograph of a hybrid human embryonic stem cell-derived cardiomyocyte-rat primary cardiomyocyte culture grown on a microelectrode array plate showing the cultured human embryonic stem cell-derived cardiomyocytic cells in the upper part as a white cluster.

FIGS. 16b-c depict an activation map of spontaneous activity showing the activation origin (red) in the rat tissue propagating to the rest of the co-culture (FIG. 16b), and simultaneous recordings from the human embryonic stem cell-derived (upper trace) and rat (lower trace) tissues (FIG. 16c). The activation map corresponds to the micrograph shown in FIG. 16a and the human and rat traces were generated from the electrodes indicated by red and green circles, respectively, in FIG. 16a.

FIGS. 16d-e depict an activation map (FIG. 16c) and simultaneous recordings (FIG. 16d) from the rat (lower trace) and human (upper trace) tissues during pacing from an electrode positioned in the rat tissue in the lower left corner area of the culture. The activation map corresponds to the micrograph shown in FIG. 16a and the human and rat traces were generated from the electrodes indicated by red and green circles, respectively, in FIG. 16a.

FIGS. 16f-g depict an activation map (FIG. 16f) and simultaneous recordings (FIG. 16g) from the rat (lower trace) and human (upper trace) tissues during pacing from an electrode positioned in the human tissue in the top, left-of-center area of the culture. The activation map corresponds to the micrograph shown in FIG. 16a and the human and rat traces were generated from the electrodes indicated by red and green circles, respectively, in FIG. 16a.

FIG. 16h is a data plot depicting optical recordings of the mechanical activity in the contracting embryoid body (top) synchronous with the electrical activity in the human (middle) and rat (bottom) tissues.

FIG. 16i-l are histograms depicting the cycle-lengths ratios between the rat and human tissues. The narrow peak at a ratio of 1 represents synchronous activity and was found during long-term recordings in all cultures at baseline (FIG. 16i), following isoproterenol administration (FIG. 16j) and in the majority of cultures following Heptanol application (FIG. 16l). In the minority of cultures studied mild gap junction uncoupling with Heptanol resulted in the appearance of episodes of 2:1 conduction blocks (FIG. 16k).

FIGS. 17a-b are photomicrographs depicting formation of abundant gap junctions between cultured human embryonic stem cell-derived cardiomyocytic cells and primary ventricular cardiomyocytes. FIG. 17a is a representative high magnification confocal fluorescence photomicrograph depicting the spatial distribution of gap junction (positive punctate Cx43 staining, green) in the co-cultures. The human embryonic stem cell-derived cardiomyocytic cells were identified via anti human HLA antibody (red cells). Note the presence of gap junctions between the cultured human embryonic stem cell-derived cardiomyocytic cells (arrow head) and at the interphase between the cultured human embryonic stem cell-derived cardiomyocytic cells and the primary ventricular cardiomyocytes (arrow). FIG. 17b is a confocal photomicrograph demonstrating transfer of lucifer yellow from the primary ventricular myocytes to the cultured human embryonic stem cell-derived cardiomyocytic cells (arrow).

FIGS. 17c-e are photomicrographs of confocal images depicting the spatial distribution of gap junctions at the interphase between the human ES cell-derived and rat cardiomyocytes. FIG. 17c—Spatial relationship between the human cells (stained in red by anti human mitochondrial antibodies) and rat cardiomyocytes (identified by ToPro3 blue staining of cell nuclei and lack of red cytoplasmatic staining); FIG. 17d—Spatial distribution of gap junctions in the hybrid cultures (positive punctuate Cx43 green staining, arrows mark the presence of gap junctions at the tissue's junction); FIG. 17e—Spatial association of the human ES derived cells, rat cells, and gap junction distribution.

FIGS. 18a-c depict typical body surface recordings (leads I, II, III) of nodal escape rhythm, ventricular rhythm, and new ventricular rhythm originating from the posterolateral area of the left ventricle (FIGS. 18a-c, respectively) following the creation of complete atrioventricular block showing complete atrioventricular dissociation.

FIGS. 19a-i depict electroanatomical mapping of the nodal escape rhythm (FIGS. 19a and 19c) and the new ventricular rhythm following cell transplantation (FIGS. 19b, 19d, 19f, 19g, 19h, 19i). Maps are shown from an anteroposterior (FIGS. 19a-b), left lateral (FIGS. 19c-d), and left posterolateral (FIGS. 19f-i) views. Note that the earliest activation during nodal rhythm (red area) originates from the superior septum and propagates to the rest of the ventricle, activating the lateral wall last (blue-purple area). In contrast, earliest activation during mapping of the new rhythm originated in the posterolateral wall with the septum being activated last. In order to correlate the electroanatomical map (FIG. 190 with pathology (FIG. 19e), the earliest activation was identified (FIG. 19f, arrow), the catheter was navigated 2 cm from this position and a radiofrequency ablation was performed (FIG. 19f, arrow head). Note the spatial correlation in pathology with the ablation site (FIG. 19e, marked by the pink needle) being exactly 2 cm away from the suture, indicating the site of cell injection. FIGS. 19g-i depict reproducibility of the electrophysiological findings. The same animal was mapped at two separate occasions and the corresponding electroanatomical maps are presented in the left posterior oblique view (FIGS. 19g-h). Note the reproducibility of the findings with the earliest activation (red) located at the posterolateral region in both case. A focal ablation was delivered during each of the two procedures at opposite sides of the earliest activation. An excellent correlation was found in pathology (FIG. 19i) with the cell injection site (blue suture) located exactly between the two ablation sites (marked by the two green needles).

FIGS. 20a-d are photomicrographs of histological analyses depicting the site of cell transplantation. FIG. 20a depicts hematoxylin and eosin (H&E) staining of transplanted cells within the recipient myocardial tissue; FIG. 20b depicts the presence of CM-Dil-labeled cells within the myocardium (red fluorescence); FIG. 20c depicts immunostaining with anti-human mitochondria antibodies (green) verifying the human phenotype of the transplanted cells. Nuclei were counterstained with ToPro3 (blue); FIG. 20d is a confocal image depicting gap junctions (immunostained for connexin-43, green) between the transplanted cells (identified by CM-Dil labeling, red) and host cells. Nuclei of host and transplanted cells are marked in blue.

FIGS. 21a-d are photomicrographs depicting changes in cell cycle activity during in-vitro maturation of ES cell-derived cardiomyocytes. Shown are confocal images depicting the results of double staining of whole EBs with anti-cTnI antibodies (FIGS. 21a, c, red labeling) and with antibodies directed against the marker of cycling cells, Ki-67 (FIGS. 21a, c, green nuclear staining). In addition, all nuclei of both cycling and noncycling cells can be viewed using the To-Pro-3 nuclear staining (FIGS. 21b, d). Note the presence of cycling myocytes (FIG. 21a, red cells with positively stained green nuclei) in early-stage EB (18 days postplating) and lack of any such cycling cells (FIG. 21c, absence of nuclear staining in all cardiomyocytes) in late-stage EBs (38 days postplating).

FIG. 22 is a bar graph depicting changes in the labeling index (percentage of Ki-67 positive nuclei) of undifferentiated stem cells, early (20 days), intermediate (30 days), and late (40 days) stage human ES cell-derived cardiomyocytes.

### DESCRIPTION OF THE PREFERRED EMBODIMENTS

The present invention is of methods of generating human cells and tissues predominantly displaying at least one characteristic associated with a cardiac phenotype, and of methods of using such cells and tissues to repair cardiac tissues, to qualify the effects of treatments on biological states and processes of cardiac cells and cardiac tissues, and to characterize biological states and processes of cardiac cells and cardiac tissues.

Specifically, the present invention can be used to generate cultured human cardiomyocytic cells and tissues displaying a broader range of human cardiac specific structures and functions than prior art cultured differentiable cell-derived cells and tissues, displaying such human specific and cardiac specific structures and functions for longer time periods than prior art cultured differentiable cell-derived cells and tissues, and displaying significant proliferative capacity. As such, the present invention can be used to generate human cardiomyocytic cells and tissues which can be used to treat human cardiac disorders with far greater efficacy than prior art cultured differentiable cell-derived cells and tissues, to test the therapeutic and toxic effects of treatments, such as pharmacological and electrical treatments, on human cardiac cells and tissues more efficiently and accurately than with prior art cultured differentiable cell-derived cells and tissues, to characterize cardiac specific gene expression, and to optimally model human cardiomyogenesis and human cardiac physiology relative to prior art cultured differentiable cell-derived cells and tissues.

The principles and operation of the present invention may be better understood with reference to the accompanying descriptions and examples.

Before explaining at least one embodiment of the invention in detail, it is to be understood that the invention is not limited in its application to the details of construction and the arrangement of the components set forth in the following description or exemplified in the Examples. The invention is capable of other embodiments or of being practiced or carried out in various ways. Also, it is to be understood that the phraseology and terminology employed herein is for the purpose of description and should not be regarded as limiting.

Death or damage of cardiomyocytes or myocardium as a result of diseases associated with cardiac cell or tissue necrosis, such as ischemic cardiac disease oftentimes leads to mortality or disability since adult cardiomyocytes are incapable of proliferating in-vivo.

Such inability of adult cardiomyocytes to proliferate represents a major stumbling block to approaches attempting to utilize in-vitro culture of adult cardiomyocytes to generate cardiomyocytic cells and tissues suitable for treating cardiac disorders, such as myocardial infarction, for testing the effects, such as the therapeutic and toxic effects of treatments, such as pharmacological and electrical treatments, on human cardiac cells and tissues, for characterizing cardiac specific gene expression, and for modeling aspects of human cardiac biology, such as human cardiac development and human cardiophysiology.

Various prior art approaches have attempted to use in-vitro culture of differentiable cells to generate cardiomyocytic cells and tissues suitable for such applications.

However, all prior art approaches have failed to provide an adequate solution for utilizing in-vitro culture of differentiable cells to generate cardiac cells and tissues suitable for treating human cardiac disorders, and for testing the pharmacological effect and toxicity of compounds on human cardiac cells and tissues.

While experimenting with cultured human embryonic stem cells, the present inventors unexpectedly uncovered that culturing human stem cells according to the method of the present invention could be used to generate cardiomyocytic cells and tissues displaying far greater cardiac specific differentiation and functionality than any prior art cultured differentiable cell-derived cells and tissues. It was further uncovered that the cardiomyocytic cells and tissues generated according to the method of the present invention displayed, for the first time by cultured differentiable cell-derived cells and tissues, a broad spectrum of human specific and cardiac specific responses, such as physiological responses, to treatments, such as pharmacological and electrical treatments. It was yet further uncovered, also for the first time by cultured differentiable cell-derived cells and tissues, that the cardiomyocytic cells and tissues of the present invention displayed human specific functional integration with primary cardiomyocytes and myocardium in-vitro and in-vivo, respectively. It was still yet further uncovered that the cardiomyocytic cells and tissues of the present invention display significant proliferative capacity.

Thus, the cells and tissues of the present invention can be used to treat human cardiac disorders, to test the effects, such as the therapeutic and toxic effects, of treatments, such as pharmacological and electrical treatments, on human cardiac cells and tissues, to characterize cardiac specific gene expression, and to model aspects of cardiac biology, such as human cardiac development and human cardiac electrophysiology, all of which optimally relative to methods employing prior art cultured differentiable cell-derived cells and tissues.

As used herein, “differentiable” cells are cells that can differentiate. Differentiable cells include, but are not limited to, non-differentiated cells, stem cells, totipotent cells, partially differentiated cells, progenitor cells, precursor cells, pluripotent cells, de-differentiated cells, and the like.

Thus, according to one aspect of the present invention, there is provided a method of generating cardiac cells and tissues predominantly displaying at least one characteristic associated with a cardiac phenotype.

As described in detail in Example 1 of the Examples section below, the method of generating the cardiac cells and tissues of the present invention is effected by partially dispersing a confluent cultured population of human stem cells into cell aggregates, preferably containing two or more cells, most preferably containing 3-20 cells.

As used herein, the phrase “cells and tissues of the present invention” refers to the cells and tissues predominantly displaying at least one characteristic associated with a cardiac phenotype of the present invention.

As used herein, a “confluent” population of cultured cells is a population of cultured cells which are adhesively interconnected, and which may or may not cover the entire available growth surface of the culture recipient in which they are cultured.

The stem cells used to generate the cells and tissues of the present invention are preferably embryonic stem cells which are grown as established cell lines, such as, for example, those described in U.S. Pat. No. 5,843,780 to J. Thomson, or by Thomson J. et al., 1998. Science 282:1145-1147. In order to ensure optimal results, single-cell clone derived embryonic stem cell lines are established and cultured according to the method of the present invention. This facilitates selection of optimal cell lines for generating the cells and tissues of the present invention, as described in Example 1 of the Examples section below.

The use of human cell lines, such as H9.2 (Amit, M. et al., 2000. Dev Biol. 227:271) according to the present invention, is greatly advantageous over prior art methods since it uniquely enables the generation of highly differentiated, highly functional, substantially proliferating human cardiomyocytic cells and tissues in-vitro.

Various techniques, such as physical or, more preferably, chemical techniques can be employed for partially dispersing a confluent population of cells. Confluent cells are preferably partially dispersed enzymatically, preferably via a non-trypsin based method, preferably using collagenase, preferably collagenase IV. Alternately, enzymes such as dispase can be employed. Application of shear flow to enzymatically treated cells may be employed to obtain a desired degree of dispersion.

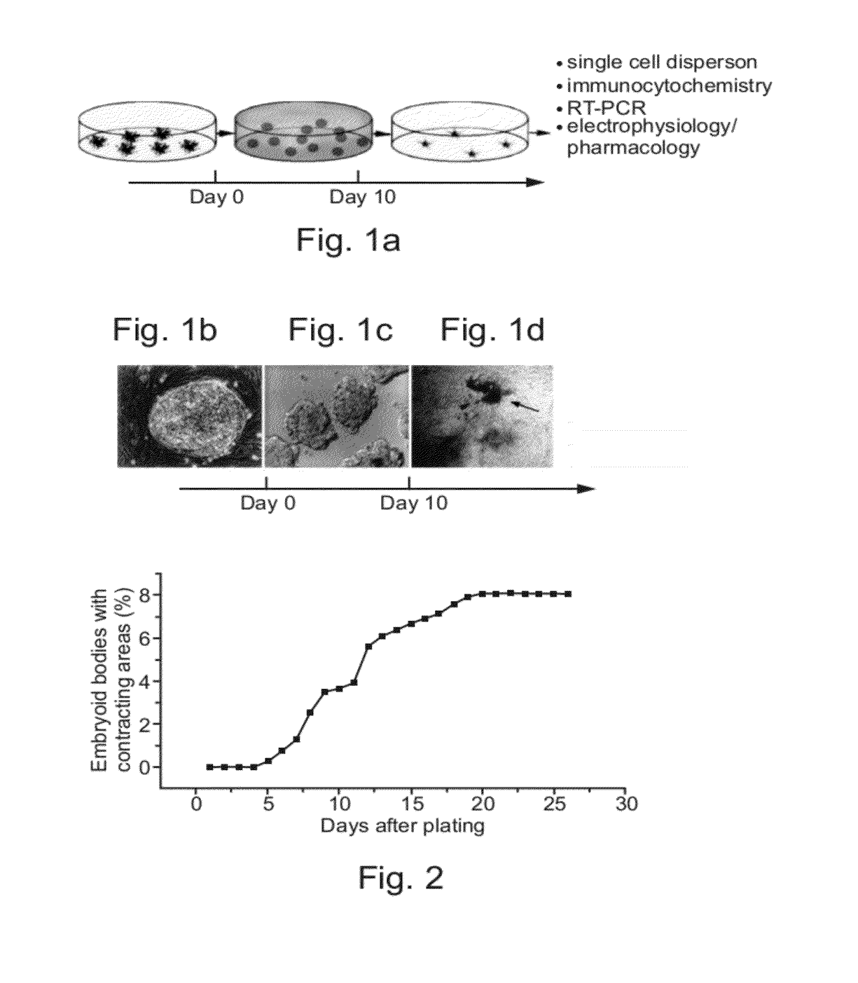

Following partial dispersion of confluent stem cells, cell aggregates are subjected to culturing conditions suitable for generating embryoid bodies, as described in further detail in Example 1 of the Examples section which follows. Embryoid bodies are easily recognized by the ordinary artisan as being coalesced embryonic stem cells displaying a characteristic structure and morphology in culture, as furthermore abundantly taught by the literature of the art. Optionally, cell aggregates are isolated, for example via microcapillary suction under a stereoscope prior to being subjected to culturing conditions suitable for generating embryoid bodies. The typical appearance of embryoid body morphology, according to the present invention, is illustrated in FIG. 1c of the Examples section below.

Culturing conditions suitable for generating embryoid bodies from cell aggregates preferably comprise inhibiting adherence of the cell aggregates to a surface, and preferably further comprise supplementing the culturing medium with serum, as described further in Example 1 of the Examples section which follows.

Inhibiting adherence of cells to a surface, such as the lower surface of a culturing recipient, is optimally effected using a culturing recipient having non-coated cell-contacting surfaces, such as, for example, non-tissue culture coated plastic bacteriology culture dishes, as described in Example 1 of the Example section below. Preventing adherence of cells to a surface of a culturing recipient can be further enhanced by culturing the cells with shaking.

Culturing of cell aggregates to induce generation of embryoid bodies is preferably effected by culturing embryoid bodies at a cell-to-surface density ranging from about 5×10⁴ to 1×10⁶ cells/cm², more preferably ranging from about 100,000 to about 500,000 cells/cm², and most preferably at a cell density of about 200,000 cells/cm². Formation of embryoid bodies can be enhanced by culturing cell aggregates under conditions allowing cell-cell contacts therebetween.

Culturing cell aggregates to generate embryoid bodies can be effected, for example, by culturing about five million cells in a volume of about 7 ml of culture medium, for example in a circular 58 millimeter diameter Petri dish, as described in Example 1 of the Examples section which follows.

Culturing of cell aggregates for generation of embryoid bodies is preferably effected for a time period ranging from about 1 day to 20 days, more preferably ranging from about 5 days to 20 days, more preferably ranging from about 5 days to 15 days, and most preferably for about 7 days to 10 days, as described in Example 1 of the Examples section below.

Prior art approaches have taught either trypsinization to disperse confluent stem cells, or the use of a hanging drop culture phase during the process of generating embryoid bodies from dispersed cells, or both. Such approaches are not adequate for generating the human cells and tissues of the present invention. For example, trypsinization of confluent human stem cells was found by the present inventors to lead to widespread karyotypic abnormalities in cells and tissues derived therefrom.

Following generation thereof, embryoid bodies are preferably subjected to culturing conditions suitable for inducing cardiac lineage differentiation in at least a portion of the cells of the embryoid bodies. Optionally, embryoid bodies are isolated, for example via microcapillary suction under a stereoscope prior to being subjected to culturing conditions suitable for inducing cardiac lineage differentiation in at least a portion of the cells of the embryoid bodies. A typical embryoid body with cells having undergone cardiac lineage differentiation via the method of the present invention is shown in FIG. 1d of the Examples section which follows.

As used herein, cells or tissues having undergone “cardiac lineage differentiation” are cells or tissues predominantly displaying at least one characteristic associated with a cardiac phenotype, as further described hereinbelow.

Culturing conditions suitable for inducing cardiac lineage differentiation preferably promote adherence of embryoid bodies to a surface, preferably the lower, cell-contacting surface of the culture recipient, and preferably further include the use of culture medium supplemented with serum.

Serum supplementation preferably comprises addition of fetal bovine serum to a volume/volume (v/v) concentration of at least about 1%, more preferably at least about 5%, more preferably at least about 10%, more preferably at least about 15%, and most preferably at least about 20%.

To promote adherence of embryoid bodies to a surface, such as the lower surface of a culture recipient, the surface is preferably coated with gelatin. Coating a surface with gelatin is preferably effected by exposing the surface to a solution containing gelatin, and incubating the surface in the presence of the solution, for example, for about sixteen hours at about 4° C., or for about thirty minutes to two hours at about 37° C. Preferably, a solution containing about 0.1% gelatin is used, as described in Example 1 of the Examples section which follows.

Alternately, adherence of embryoid bodies to a surface can be promoted, for example, by culturing the embryoid bodies in a recipient having cell-contacting surfaces coated with an extracellular matrix (ECM) component, such as fibronectin, or with a mixture of extracellular matrix components, such as Matrigel™, or by culturing the embryoid bodies in tissue culture coated tissue culture dishes, such as Falcon™ tissue culture-coated culture dishes.

Culturing embryoid bodies to induce cardiac lineage differentiation in at least a portion of the cells of the embryoid bodies is preferably effected for a duration of at least: 1 day, more preferably 2 days, more preferably 3 days, more preferably 4 days, more preferably 5 days, more preferably 6 days, more preferably 7 days, more preferably 8 days, more preferably 9 days, more preferably 10 days, more preferably 11 days, more preferably 12 days, more preferably 13 days, more preferably 14 days, more preferably 15 days, more preferably 16 days, more preferably 17 days, more preferably 18 days, more preferably 19 days, more preferably 20 days, more preferably 21 days, more preferably 22 days, more preferably 23 days, more preferably 24 days, more preferably 25 days, more preferably 26 days, more preferably 27 days, more preferably 28 days, more preferably 29 days, more preferably 30 days, more preferably 31 days, more preferably 32 days, more preferably 33 days, more preferably 34 days, more preferably 35 days, more preferably 36 days, more preferably 37 days, more preferably 38 days, more preferably 39 days, more preferably 40 days, more preferably 41 days, more preferably 42 days, more preferably 43 days, more preferably 44 days, more preferably 45 days, more preferably 46 days, more preferably 47 days, more preferably 48 days, more preferably 49 days, more preferably 50 days, more preferably 51 days, more preferably 52 days, more preferably 53 days, more preferably 54 days, more preferably 55 days, more preferably 56 days, more preferably 57 days, more preferably 58 days, more preferably 59 days, and most preferably 60 days.

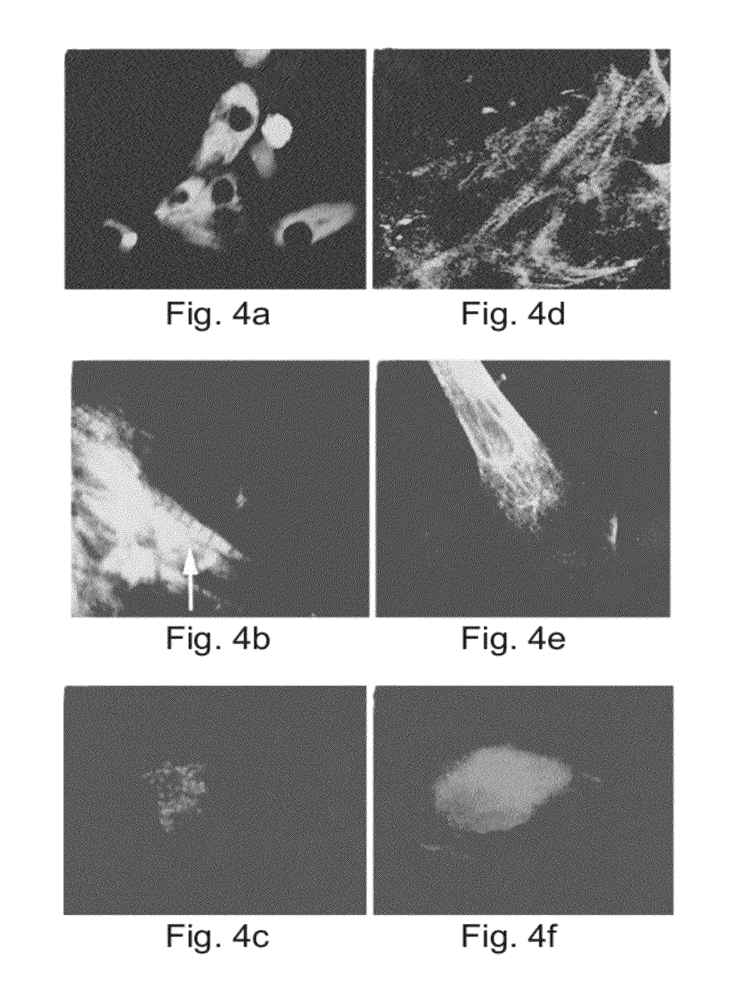

The time period during which embryoid bodies are cultured can be varied according to the cardiac differentiation features which are desired. For example, as described in FIG. 3 of the Examples section below, culturing embryoid bodies for a time period ranging from 10 days to 27 days can be used to generate cells displaying increasingly mature myofibrillar organization. As described in Example 4 of the Examples section below, culturing embryoid bodies for 7-20 days can be used to generate optimally proliferating cardiomyocytic cells and tissues. In general, as described in Example 4 of the following Examples section, the shorter the duration of embryoid body culture, the greater the proliferative capacity of the cardiomyocytic cells and tissues generated.

Culturing conditions suitable for inducing cardiac lineage differentiation in at least a portion of the cells of the embryoid bodies preferably comprise supplementing the culture medium with dimethyl sulfoxide (DMSO), preferably 0.75 percent (v/v) dimethyl sulfoxide. As described in the Examples section which follows, this can be used to increase the percentage of embryoid bodies with cells displaying cardiac lineage differentiation by about 20%.

As described in Example 1 of the Examples section hereinbelow it was uncovered by the present inventors that not all embryoid bodies cultured under conditions suitable for inducing cardiac lineage differentiation only contain cells displaying cardiac lineage differentiation. Thus, the method according to this aspect of the present invention preferably further comprises screening and identifying cells and tissues displaying cardiac lineage differentiation within the cultured embryoid bodies.

According to the method of the present invention, screening is performed by a method enabling detection of at least one characteristic associated with a cardiac phenotype, as described hereinbelow, for example via detection of cardiac specific mechanical contraction, detection of cardiac specific structures, detection of cardiac specific proteins, detection of cardiac specific RNAs, detection of cardiac specific electrical activity, and detection of cardiac specific changes in the intracellular concentration of a physiological ion.

Preferably, screening the cells and tissues of the present invention comprises detection of mechanical contraction. Cardiac specific mechanical contraction can often be identified in outgrowths of embryoid bodies such as that highlighted in FIG. 1d of the Examples section below.

Various techniques can be used to detect each of cardiac specific mechanical contraction, cardiac specific structures, cardiac specific proteins, cardiac specific RNAs, cardiac specific electrical activity, and cardiac specific changes in the intracellular concentration of a physiological ion.

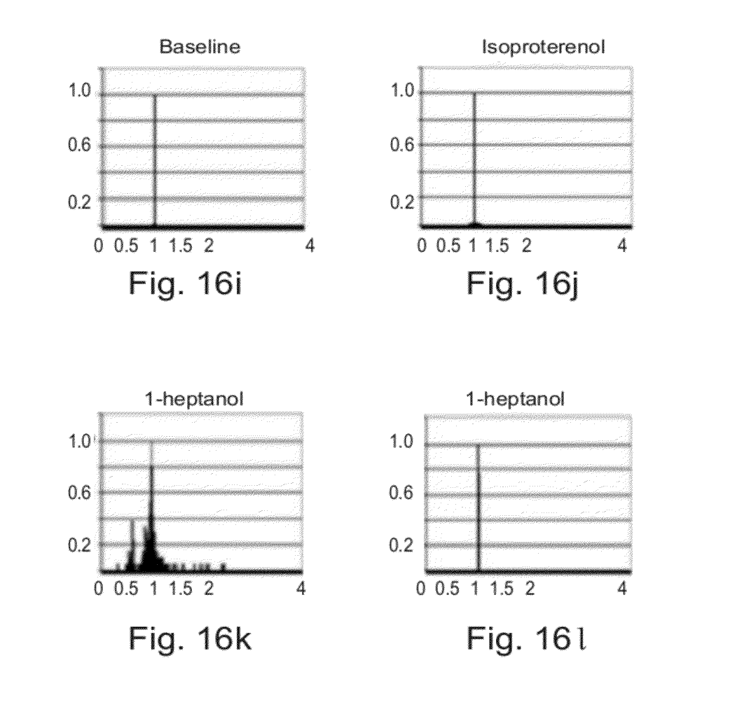

Detection of cardiac specific mechanical contraction is preferably effected visually using an optical microscope. Alternately, such detection can be effected and recorded using a microscope equipped with a suitable automated motion detection system using, for example, a photodiode, as described in Example 3, and as demonstrated in FIG. 16h of the Examples section below.

Detection of cardiac specific structures is preferably performed via light microscopy, fluorescence affinity labeling and fluorescence microscopy, or electron microscopy, depending on the type of structure whose detection is desired. Light microscopy can be used to detect various cardiac specific structures, such as mononuclear cells, about 10-30 μm in diameter, with round or rod-shaped morphology characteristic of immature cardiomyocytes, as described in Example 1 of the Examples section which follows. For resolution of details, phase contrast microscopy may be employed, as described in the Examples section.

To detect cardiac specific structures via electron microscopy, the transmission electron microscopy protocol described in detail in Example 1 of the following Examples section is preferably employed. Alternately, numerous suitable protocols which are available in the literature can also be utilized. Electron microscopy can be used to detect various cardiac specific structures, such as sarcomeres, Z-bands, Z-bodies, intercalated discs, gap junctions, desmosomes, fibrillar bundles, fibrillar bundle striations, and myocytic syncytia, as demonstrated in FIG. 3 of the Examples section which follows.

To detect cardiac specific structures via fluorescence affinity labeling and fluorescence microscopy, such structures may be fluorescently labeled via a molecule, commonly an antibody, which specifically binds such a structure, and which is either directly or indirectly conjugated to a fluorophore. Automated quantitation of such structures can be performed using appropriate detection and computation systems, for example, as described and as demonstrated in the Examples section below. When using an antibody as an affinity labeling reagent, this technique may be referred to as immunocytochemistry or immunohistochemistry. Using the appropriate affinity labeling reagents and fluorophores, fluorescence affinity labeling and fluorescence microscopy can be used to detect various cardiac specific structures, including, but not limited to, sarcomeres, gap junctions, fibrillar bundles, fibrillar bundle striations, and myocytic syncytia, as is described and illustrated in the Examples section which follows.

Detection of cardiac specific proteins is preferably effected via fluorescence affinity labeling and fluorescence microscopy, as described hereinabove for detection of a cardiac specific structure. Alternately, techniques such as Western immunoblotting or hybridization micro arrays (“protein chips”) may be employed. As is illustrated in the Examples section below, fluorescence affinity labeling and fluorescence microscopy can be used to detect cardiac specific proteins, such as cardiac α-myosin heavy chain, cardiac β-myosin heavy chain, atrial natriuretic peptide, cardiac troponin I, desmin and connexin-43.

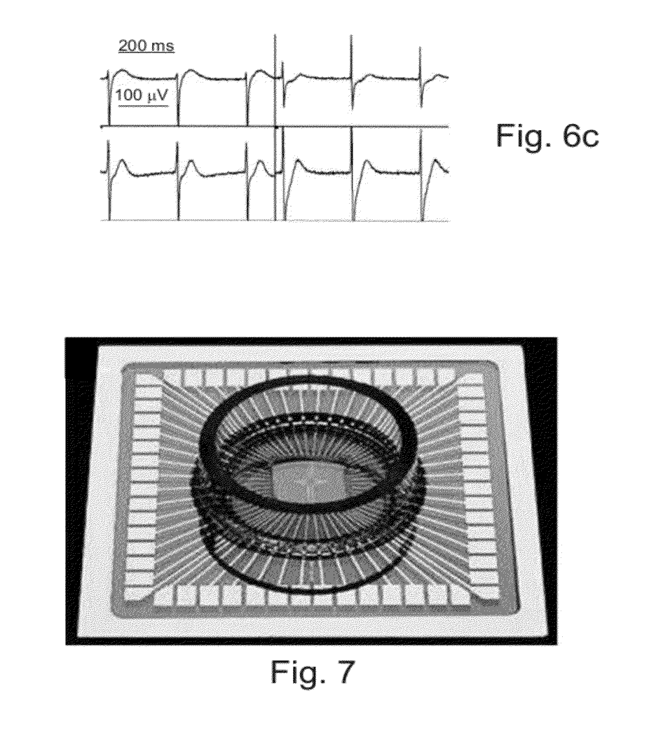

Detection of cardiac specific RNAs is preferably effected using RT-PCR, for example using the protocol and primers described Example 1 of the Examples section below. Alternately, other commonly used methods, such as hybridization microarray (“RNA chip”) or Northern blotting, may be employed. RT-PCR can be used to detect cardiac specific RNAs encoding essentially any protein, including cardiac α-myosin heavy chain, cardiac β-myosin heavy chain, α-actinin, cardiac troponin I, cardiac troponin T, GATA-4, Nkx2.5, MLC-2A, MLC-2V, atrial myosin light chain, ventricular myosin light chain, and connexin-43, as is demonstrated in FIG. 5 of the Examples section.

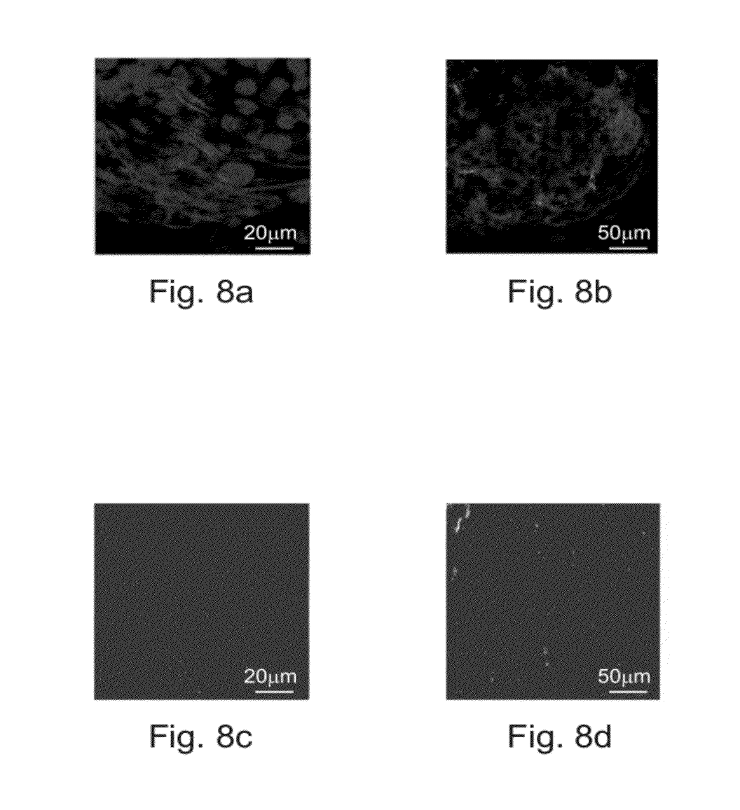

Detection of cardiac specific changes in the intracellular concentration of a physiological ion, such as calcium, is preferably effected using assays based on fluorescent ion binding dyes such as the fura-2 calcium binding dye (for example, refer to Brixius, K. et al., 1997. J Appl Physiol. 83:652). Such assays can be advantageously used to detect changes in the intracellular concentration of calcium ions, such as calcium transients, as is illustrated in FIGS. 6a-b of the Examples section below.

Detection of cardiac specific electrical activity of the cells and tissues of the present invention is preferably effected by monitoring the electrical activity thereof via a multielectrode array. Suitable multielectrode arrays may be obtained from Multi Channel Systems, Reutlingen, Germany, and are preferably employed as described in the following Examples section.

Preferably, the multielectrode array utilized by the present invention is a two-dimentional orthogonal array which includes 60 or more electrodes positioned 100 ₗam or less apart.

Preferably, the multielectrode array is configured to obtain data characterizing cardiac specific electrical activity with a frequency greater than a range selected from 1-25 kHz.

To detect cardiac specific electrical activity in the cells and tissues of the present invention, the latter can be advantageously cultured, under conditions suitable for inducing cardiac differentiation directly on a multielectrode array, thereby conveniently enabling monitoring the electrical activity such cells and tissues. Regions of embryoid bodies displaying cardiac differentiation, preferably in the form of cardiac specific mechanical contraction, can be advantageously microdissected from embryoid bodies and cultured on microelectrode arrays, as described in Example 1 of the Examples section, below. Such direct culturing on a multielectrode array advantageously enables the monitoring of long-term electrical activity of the cells and tissues of the present invention, as described in the Examples section which follows.

Monitoring electrical activity in the cells and tissues of the present invention can be used to provide many different types of important and novel information regarding electrical activity of cells and tissues of the present invention. For example, such monitoring can be used to monitor electrical activity individually at each electrode, or more advantageously, such monitoring can be used to generate electrical activity propagation maps, also termed herein “activation maps”, depicting electrical activity as a function of local activation time at each electrode, for example in the form of a color-coded gradient. Such activation maps can be used to depict conduction velocity and conduction directionality of propagative electrical activity, preferably in the form of conduction velocity vectors, of electrical activity propagation over an area of the microelectrode array, as is illustrated, for example in FIG. 9 of the following Examples section.

According to a preferred embodiment of the present invention, the present method further comprises isolating the cells and tissues of the present invention from embryoid bodies. Such isolation is preferably performed by mechanical dissection of cells and tissues displaying the desired characteristic with a pulled-glass micropipette or microscalpel, as described in the Examples section which follows. Alternately, cells and tissues displaying a cell surface molecule which can be specifically bound by a reagent may be isolated by fluorescence-activated cell sorting (FACS).

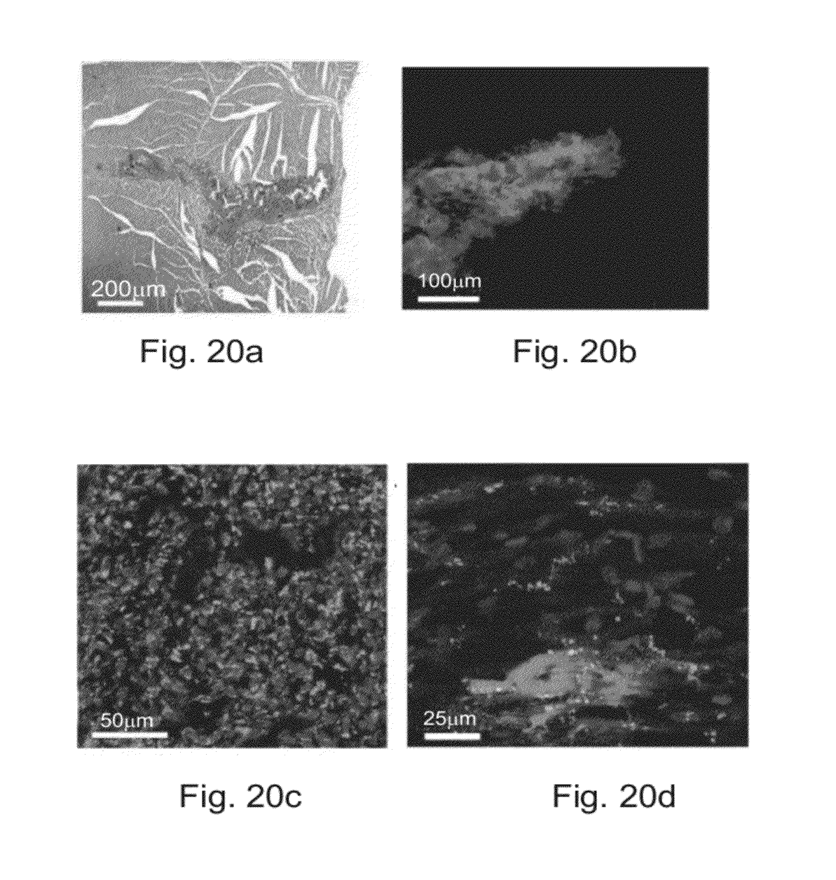

Since the proliferative capacity of the cells and tissues of the present invention may be essential to a given use thereof, for example, to repair cardiac tissue, or to test the effects of a treatment on growth, differentiation, or electric activity of cardiac cells and tissues, the method according to this aspect of the present invention may advantageously further comprise screening and optionally isolating cells and tissues substantially displaying proliferation. Determining the proliferative capacity of cells can be performed by numerous standard techniques. Preferably, determination of proliferation is effected via ³[H]-thymidine uptake assay and autoradiographic detection of such uptake. Alternately, colorimetric assays employing metabolic dyes such as XTT, or direct cell counting may be employed to ascertain proliferative capacity. In addition, as is shown in FIGS. 21a-d and in Example 4 of the Examples section which follows, the proliferation capacity can be evaluated via the expression of cell cycle markers such as Ki-67.

Thus, the method of the present invention is unique over prior art methods in that it enables generation of proliferating cells and tissues predominantly displaying characteristics associated with a cardiac phenotype by in-vitro culture of differentiable cells. Furthermore, since the method of the present invention can be used to generate such cells and tissues in-vitro, the method of the present invention possesses the unique and extremely useful capacity to generate essentially unlimited numbers of such cells and tissues.

As is illustrated in the Examples section which follows, the cells and tissues generated using the methodology of the present invention display characteristics associated with a cardiac phenotype including, but not limited to, cardiac specific mechanical contraction, cardiac specific structures, expression of cardiac specific RNAs, expression of cardiac specific proteins, cardiac specific changes in the intracellular concentration of a physiological ion, and cardiac specific electrical activity, as further described hereinbelow.

Preferably the cardiac phenotype is a cardiomyocytic phenotype. As evidenced by the characteristic spectrum of structures and functions displayed by the cells and tissues of the present invention described below and in the Examples section below, the cells and tissues of the present invention clearly display a highly functional, highly differentiated cardiomyocytic phenotype.

Preferably, the cells and tissues of the present invention display cardiac specific mechanical contraction, more preferably in combination with at least some, and most preferably in combination with at least all of the following characteristics associated with a cardiac phenotype: cardiac specific structures, expression of cardiac specific RNAs, expression of cardiac specific proteins, cardiac specific changes in the intracellular concentration of a physiological ion, and cardiac specific electrical activity.

The cells and tissues of the present invention display types of cardiac specific mechanical contraction including, but not limited to, spontaneous mechanical contraction, rhythmic mechanical contraction, synchronous mechanical contraction, and propagative mechanical contraction.

As is shown in FIG. 16h of the Examples section below, the cells and tissues of the present invention can be generated displaying spontaneous mechanical contraction and rhythmic mechanical contraction. As observed visually, as described in the Examples section below, and as can be deduced from FIG. 9c of the following Examples section, the cells and tissues of the present invention can be generated displaying synchronized mechanical contraction and propagative mechanical contraction.

The rhythmic mechanical contraction of the cells and tissues of the present invention exhibit cardiac specific chronotropic responses to pharmacological agents. Preferably, the cells and tissues of the present invention display chronotropic responses to catecholamines such as isoproterenol, carbamylcholine, adenylate cyclase activators such as forskolin, phosphodiesterase inhibitors such as IBMX, atropine, and diltiazem hydrochloride. As is shown in the Examples section which follows, the cells and tissues of the present invention display cardiac specific increases in mechanical contraction rhythm in response to forskolin, the phosphodiesterase inhibitor IBMX, and isoproterenol, and cardiac specific, decreases in mechanical contraction rhythm in response to muscarinic antagonists such as carbamylcholine, and atropine mediated reversal of decreases in mechanical contraction rhythm in response to muscarinic antagonists such as carbamylcholine.

The cells and tissues of the present invention further display types of cardiac specific structures including, for example, sarcomeres, Z-bands, Z-bodies, intercalated discs, gap junctions, desmosomes, fibrillar bundles, fibrillar bundle striations, myocytic syncytia, and mononuclear cells, about 10-30 μm in diameter, with round or rod-shaped morphology characteristic of immature cardiomyocytes, as described in Example 1 of the Examples section which follows.

The cells and tissues of the present invention further display expression of cardiac specific RNAs encoding proteins such as, for example, cardiac α-myosin heavy chain, cardiac β-myosin heavy chain, α-actinin, cardiac troponin I, cardiac troponin T, GATA-4, Nkx2.5, MLC-2A, MLC-2V, atrial myosin light chain, ventricular myosin light chain, and connexin-43, as is demonstrated in FIG. 5 of the Examples section.

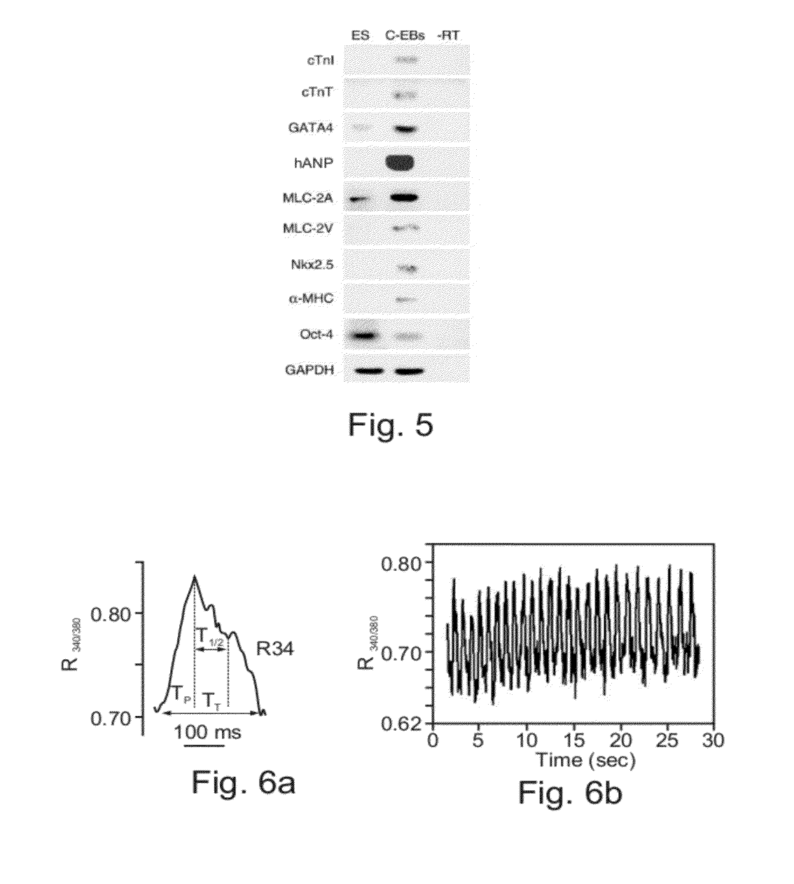

The cells and tissues of the present invention still further display cardiac specific proteins including, for example, cardiac α- and β-myosin heavy chains, desmin, atrial natriuretic peptide, cardiac troponin I, and connexin-43, as is illustrated in FIGS. 4b, 4e, 4f, 8b and 20d, respectively, of the Examples section below.

The cells and tissues of the present invention additionally display types of cardiac specific electrical activity such as, for example, spontaneous electrical activity, rhythmic electrical activity, and synchronized/propagative electrical activity, as is demonstrated in FIGS. 9b, 6c, and 9c, respectively, of the following Examples section.

The cells and tissues of the present invention yet additionally display types of cardiac specific changes in the intracellular concentration of a physiological ion including, for example, cardiac specific changes in the intracellular concentration of Ca²⁺, for example in the form of calcium transients, as is illustrated in FIGS. 6a-b of the Examples section below.

Thus, as is shown in the Examples section which follows, the cells and tissues of the present invention display cardiac specific mechanical contraction, cardiac specific structures, expression of cardiac specific RNAs, expression of cardiac specific proteins, cardiac specific changes in the intracellular concentration of a physiological ion, and/or cardiac specific electrical activity. The cells and tissues of the present invention therefore uniquely display a broad spectrum of human specific and cardiac specific structural and functional characteristics relative to prior art cultured differentiable cell-derived cells and tissues.

The propagative electrical activity of the cells and tissues of the present invention exhibit cardiac specific responses to pharmacological agents. Preferably, the cells and tissues of the present invention can display slowing of conduction in response to exposure to suitable concentrations of fast sodium channel blockers, such as 1-heptanol, tetrodotoxin (TTX), and to suitable increases in extracellular potassium ion, as is described in Example 2 of the Examples section which follows.

As is described in Example 1 of the Examples section which follows, the cells and tissues of the present invention display characteristics associated with a cardiac phenotype for at least 60 days. This extended period of time during which the cells and tissues of the present invention display such characteristics represents a large improvement over that of cells and tissues generated via prior art methods which do not, for example, have the capacity to display highly functional mechanical contraction for extended periods of time.

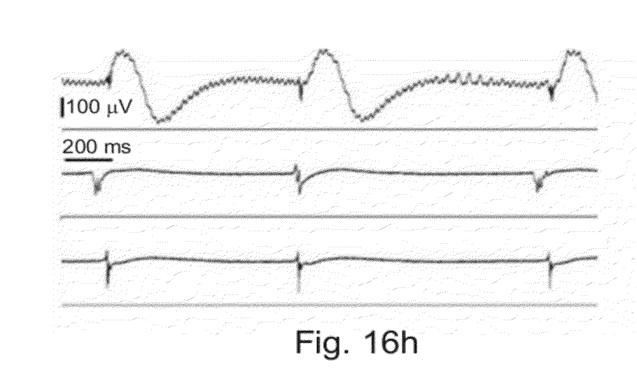

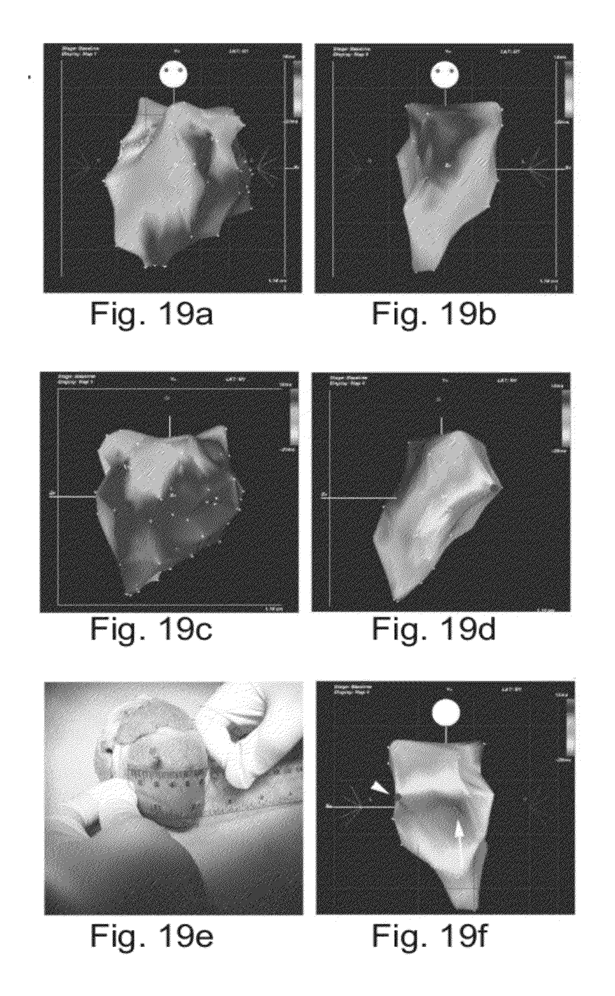

The above described characteristics of the cells and tissues of the present invention imply that these cells and tissues have the capacity to functionally integrate with primary cardiac cells and tissues in-vitro and in-vivo. Preferably, such functional integration comprises formation of an integrated excitatory/excitable cardiomyocytic syncytium with primary cardiac cell and tissues. As is shown in FIGS. 15a and 19c-d, respectively, of the Examples section which follows, the cells and tissues of the present invention have the capacity to form an integrated excitable/excitatory cardiomyocytic syncytium with primary ventricular cardiomyocytes in-vitro and with myocardium in-vivo.

The cells and tissues of the present invention also display substantial proliferative qualities. Preferably, such proliferation is characterized by a proliferation index of at least about 10%, more preferably at least about 20%, yet more preferably at least about 30%, still more preferably at least about 40%, yet still more preferably about 50% and most preferably at least about 60%. As shown in Example 4 of the following Examples section, the cells and tissues of the present invention may display a proliferative index of about 60%.

Thus, the method of the present invention can be used to generate highly differentiated, highly functional, proliferating human cells and tissues displaying a broad range of cardiac functional and structural characteristics, which cells and tissues having a potent capacity of functionally integrating with primary cardiac tissues, such as myocardium, in-vivo.

As such, the isolated cells and tissues generated by the method of the present invention can be optimally employed, for example, in various therapeutic, pharmacological, analytic, and modeling applications discussed herein.

Thus, according to another aspect of the present invention, there is provided a method of repairing cardiac tissue in a human subject.

The method is preferably applied to repair cardiac tissue in a human subject having a cardiac disorder so as to thereby treat the disorder. The method can also be applied to repair cardiac tissue susceptible to be associated with future onset or development of a cardiac disorder so as to thereby inhibit such onset or development.

The present invention can be advantageously used to treat disorders associated with, for example, necrotic, apoptotic, damaged, dysfunctional or morphologically abnormal myocardium. Such disorders include, but are not limited to, ischemic heart disease, cardiac infarction, rheumatic heart disease, endocarditis, autoimmune cardiac disease, valvular heart disease, congenital heart disorders, cardiac rhythm disorders, impaired myocardial conductivity and cardiac insufficiency. Since the majority of cardiac diseases involve necrotic, apoptotic, damaged, dysfunctional or morphologically abnormal myocardium, and since the cells and tissues of the present invention display a highly differentiated, highly functional, and proliferating cardiomyocytic phenotype, the method of repairing cardiac tissue of the present invention can be used to treat the majority of instances of cardiac disorders.

According to a preferred embodiment, the method according to this aspect of the present invention can be advantageously used to efficiently reverse, inhibit or prevent cardiac damage caused by ischemia resulting from myocardial infarction.

According to another embodiment, the method according to this aspect of the present invention can be used to treat cardiac disorders characterized by abnormal cardiac rhythm, such as, for example, cardiac arrhythmia.

As used herein the phrase “cardiac arrhythmia” refers to any variation from the normal rhythm of the heart beat, including, but not limited to, sinus arrhythmia, premature beat, heart block, atrial fibrillation, atrial flutter, pulsus alternans and paroxysmal tachycardia.

According to another embodiment, the method according to this aspect of the present invention can be used to treat impaired cardiac function resulting from tissue loss or dysfunction that occur at critical sites in the electrical conduction system of the heart, that may lead to inefficient rhythm initiation or impulse conduction resulting in abnormalities in heart rate.

As described in the Examples section which follows, the cells and tissues of the present invention form excitable/excitatory cardiomyocytic syncytia with primary cardiac cells and tissues in-vitro and with myocardium in-vivo, and furthermore display pace-making capacity in the context of such syncytia. Thus, the cells and tissues of the present invention can be used, for example, to provide pace-making activity to impaired myocardium and to treat an abnormal cardiac rhythm when administered to impaired cardiac tissue in-vivo.

The method according to this aspect of the present invention is effected by administering a therapeutically effective dose of the cells and/or tissues of the present invention to the heart of the subject, preferably by injection into the heart of the subject. As used herein, a therapeutically effective dose is an amount sufficient to effect a beneficial or desired clinical result, which dose could be administered in one or more administrations. Preferably, a single administration is employed. The injection can be administered into various regions of the heart, depending on the type of cardiac tissue repair required. Administration is preferably intramyocardial. Intramyocardial administration is particularly advantageous for repairing cardiac tissue in a subject having a cardiac disorder characterized by cardiac arrhythmia, impaired cardiac conducting tissue or myocardial ischemia.

Such administration directly into cardiac tissue ensures that the administered cells/tissues will not be lost due to the contracting movements of the heart.

The cells and tissues of the present invention can be administered via transendocardial or transepicardial injection, depending on the type of cardiac tissue repair being effected, and the physiological context in which the cardiac repair is effected. This allows the administered cells or tissues to penetrate the protective layers surrounding membrane of the myocardium.

Preferably, a catheter-based approach is used to deliver a transendocardial injection. The use of a catheter precludes more invasive methods of delivery wherein the opening of the chest cavity would be necessitated. As one skilled in the art is aware, optimum time of recovery would be allowed by the more minimally invasive procedure, which as outlined here, includes a catheter approach.

In Example 3 of the Examples section which follows, a dose of 20-40 contracting embryoid bodies (each embryoid body containing approximately 20,000 cells) was injected into cardiac tissue of impaired hearts of pigs weighing twenty to thirty kilograms. Such a dose of injected cells restored cardiac functionality, by forming a functionally integrated excitable/excitatory cardiomyocytic syncytium and providing pacemaking activity thereto. Thus, the results presented herein clearly provide and approach for repairing cardiac tissue in a subject having a cardiac disorder associated with cardiac arrhythmia or ischemic myocardium.

In addition, as is shown in Example 1 of the Examples section which follows, the positive and negative chronotropic responses to isoproterenol and carbamylcholine demonstrate the presence of functional adrenergic and cholinergic receptors, respectively, in the pacemaker cells/tissue of the present invention.

Thus, the cultured cardiomyocytes of the present invention can be utilized to regulate the contraction rate of a heart in response to physiological or metabolic state of the recipient individual, thereby serving as a biological pacemaker.

A biological pacemaker is superior to all prior art synthetic pacemakers, since cells of a biological pacemaker can respond to both metabolic and physiological changes in the body much in the same way as a natural pacemaker.

In the case of repairing cardiac tissue in a subject having a cardiac disorder characterized by cardiac arrhythmia, electrophysiological mapping of the heart and/or inactivation of cardiac tissue by radiofrequency treatment may be advantageously performed in combination with administration of the cells and tissues of the present invention if needed. As described in detail in Example 3 of the Examples section, the cells and tissues of the present invention can be used to confer pace-making activity to myocardium inactivated by radiofrequency treatment.

To repair cardiac tissue damaged by ischemia, for example due to a cardiac infarct, the cells and tissues of the present invention are preferably administered to the border area of the infarct. As one skilled in the art would be aware, the infarcted area is grossly visible, allowing such specific localization of application of therapeutic cells to be possible. The precise determination and timing of an effective dose in this particular case may depend, for example, on the size of an infarct, and the time elapsed following onset of myocardial ischemia.

Preferably, administration of the cells/tissues of the present invention for repair of damaged myocardium is effected following sufficient reduction of inflammation of affected cardiac tissues and prior to formation of excessive scar tissue.

As mentioned hereinabove, the present invention can be used to generate cardiomyocytic cells and tissues displaying a desired proliferative capacity, thus cells and tissues are preferably selected displaying a suitable proliferative capacity for administration, depending on the type of cardiac tissue repair being effected. Administration of highly proliferative cells may be particularly advantageous for reversing myocardial damage resulting from ischemia since, as previously described, it is the essential inability of normal adult cardiomyocytes to proliferate which causes the irreversibility of ischemia induced myocardial damage.

Since porcine models are widely considered to be excellent models for human therapeutic protocols and since such models have been widely employed and characterized, it is well within the grasp of the ordinarily skilled artisan to determine a therapeutically effective dose for a human based on the guidance provided herein, and on that provided by the extensive literature of the art.

Preferably, determination of an effective dose can further be based on factors individual to each subject, including, for example, weight, age, physiological status, medical history, and parameters related to the cardiac disorder, such as, for example, infarct size and elapsed time following onset of ischemia. One skilled in the art, specifically a cardiologist, would be able to determine the number of the cells and tissues of the present invention that would constitute an effective dose, and the optimal mode of administration thereof without undue experimentation.

It will be recognized by the skilled practitioner that when administering non-syngeneic cells or tissues to a subject, there is routinely immune rejection of such cells or tissues by the subject. Thus, the method of the present invention preferably further comprises treating the subject with an immunosuppressive regimen, preferably prior to such administration, so as to inhibit such rejection. Immunosuppressive protocols for inhibiting allogeneic graft rejection, for example via administration of cyclosporin A, immunosuppressive antibodies, and the like are widespread and standard practice in the clinic.

The treatment method of this aspect of the present invention can be effected by administering the cells and tissues of the present invention as isolated cells or tissues or as an engineered cardiomyocytic tissue having a therapeutically useful two or three-dimensional form.

Engineered cardiomyocytic tissues can be generated via standard tissue engineering techniques, for example by seeding a tissue engineering scaffold having the designed form with the cells and tissues of the present invention and culturing the seeded scaffold under conditions enabling colonization of the scaffold by the seeded cells and tissues, thereby enabling the generation of the formed cardiac tissue. The formed tissue is then administered to the cardiac tissue of the recipient, for example using standard surgical implantation techniques. Suitable scaffolds may be generated, for example, using biocompatible, biodegradable polymer fibers or foams, comprising extracellular matrix components, such as, laminins, collagen, fibronectin, etc. Detailed guidelines for generating or obtaining suitable scaffolds, culturing such scaffolds and therapeutically implanting such scaffolds are available in the literature of the art (for example, refer to Kim S S. and Vacanti J P., 1999. Semin Pediatr Surg. 8:119; U.S. Pat. No. 6,387,369 to Osiris Therapeutics, Inc.; U.S. Pat. App. No. US20020094573A1 to Bell E.).

Thus, the method of repairing cardiac tissues of the present invention, by virtue of its uniqueness in utilizing in-vitro culture of human differentiable cells to generate essentially unlimited numbers of highly differentiated, highly functional and proliferating cardiomyocytic cells and tissues is far superior to all prior art methods.

According to yet another aspect of the present invention, there is provided a method of characterizing biological states and biological processes of cardiac cells and cardiac tissues.

As used herein, “characterizing” a state or process refers to generating data or obtaining information defining or describing such a state or process.

Since the cells and tissues of the present invention display an exceptionally broad spectrum of human and cardiac specific characteristics, and since the present invention can be used to generate essentially unlimited quantities of such cells and tissues, such cells and tissues can be used to accurately model in-vitro and in-vivo a very broad range of cardiac specific biological states and processes. Furthermore, since the method of the present invention can be used to generate cells and tissues displaying high cardiac functionality for at least up to 60 days in culture, the present invention can advantageously be employed to characterize cardiac specific biological states and processes over extended periods of time. Thus, the method of the present invention can uniquely be used to characterize substantially long-term processes such as, for example, human cardiac physiology and human cardiac development. For example, as is shown in FIG. 4 of the Examples section below, the method can be used to characterize the long-term development of cardiac cells and tissues in-vitro.

As described in detail hereinabove and in the Examples section hereinbelow, the method can be used to characterize biological states and processes corresponding to all of the characteristics associated with a cardiac phenotype displayed by the cells and tissues of the present invention described hereinabove, such as cardiac specific mechanical contraction, cardiac specific structures, expression of cardiac specific RNAs, expression of cardiac specific proteins, cardiac specific changes in the intracellular concentration of a physiological ion, cardiomyogenesis and most preferably cardiac specific electrical activity.

As used herein, the word “cardiomyogenesis” refers to any process of growth and/or differentiation of cardiac cells or tissues.

By virtue of enabling characterization of gene transcription in the cells and tissues of the present invention, the method according to this aspect of the present invention can be advantageously used to obtain comprehensive profiles of cardiac specific gene expression, such as for example gene expression profiles related to cardiomyocytic growth and differentiation, or gene expression profiles related to a characteristic associated with a cardiac phenotype, as described hereinabove.

Thus, the method according to this aspect of the present invention can be advantageously employed to identify novel cardiac specific genes, such as novel cardiac specific genes related to growth and differentiation of cardiac cells and tissues, or novel cardiac specific genes associated with a characteristic associated with a cardiac phenotype, as described hereinabove.

Due to the proliferative capacity of the cells and tissues of the present invention, the method according to this aspect of the present invention may be advantageously employed to characterize biological states and processes related to growth and differentiation of cardiac cells.

The method of characterizing biological states and processes is preferably effected by generating the cells and tissues of the present invention, essentially as described hereinabove and in the Examples section below, and obtaining data characterizing selected biological states or biological processes in such cells and tissues. Obtaining such data is effected essentially as described hereinabove for screening characteristics associated with a cardiac phenotype in such cells and tissues.

As described hereinabove and in the Examples section below, the cardiac cells and tissues of the present invention possess the capacity to functionally integrate with primary cardiac cells and tissues in-vitro or with primary cardiac tissue in-vivo. Thus, the method according to this aspect of the present invention can further comprise co-culturing the cells or tissues of the present invention with primary cardiac cells or primary cardiac tissue prior to obtaining data characterizing a biological states or process of such cells and tissues, as described in the Examples section which follows.

As is illustrated in FIG. 15b and described in the Examples section below, biological states and processes characterized by cardiac specific chronotropic responses can be induced in integrated myocytic syncytia comprising the cells and tissues of the present invention and primary cardiomyocytes.

Alternately, the method may include transplanting the cells or tissues of the present invention into cardiac tissue of a recipient prior to obtaining data characterizing a biological state or process of such cells and tissues. Preferably the recipient is a swine. Such co-culturing or transplanting can be used to characterize biological states and processes of cardiac cells and tissues to which the cells and tissues of the present invention have been applied. This type of characterization provides important information, for example, for optimizing and testing strategies for therapeutic transplantation of cardiac cells and tissues. As is shown in Example 3 of the Examples section which follows, transplantation can be used to characterize development of the cardiac rhythm of the cells and tissues of the present invention in an in-vivo transplantation context.

The method according to this aspect of the present invention may further comprise a step of inducing such biological states and processes in the cells and tissues of the present invention prior to obtaining data characterizing a biological state or process. As described in the Examples section and hereinabove, the cells and tissues of the present invention display various cardiac specific biological states or processes in response to pharmacological and electrical treatments. Preferably, the method is used to induce abnormal states and processes in the cells and tissues of the present invention, thereby enabling characterization of cardiac disorders associated with such abnormal states. As described in detail hereinabove and the following Examples section, various treatments can be used to induce cardiac specific chronotropic responses or cardiac specific changes in conduction of propagative electrical activity. For example, as is demonstrated in Example 2 of the following Examples section, increasing extracellular potassium concentration or treatment with tetrodotoxin or 1-heptanol can be used to induce slowing of propagative electrical activity conduction in the cells and tissues of the present invention.

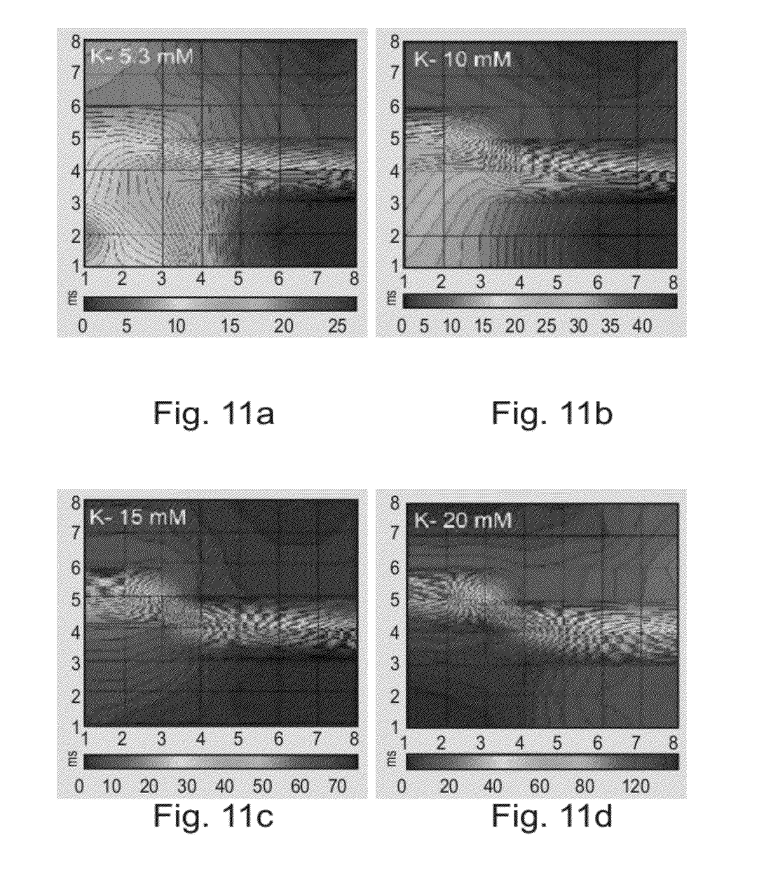

Alternately, the cells and tissues of the present invention can be screened for cells and tissues displaying selected biological states and/or processes such as abnormal biological states and processes. As is illustrated in FIG. 10b of the Examples section below, slow conduction of propagative electrical activity can be observed in embryoid bodies screened for the presence of foci of the cells and tissues of the present invention comprising two regions connected by a narrow strand of such cells and tissues, which narrow strand exhibiting the slowing of conduction.

Thus, the method according to this aspect of the present invention can be advantageously used to model various cardiac biological states and processes, including abnormal cardiac biological states and processes. Such modeling can be used, for example, for optimizing and testing strategies for therapy of cardiac disorders. As previously described hereinabove and in the Examples section below, atropine treatment can be shown to lead to the expected cardiac specific reversal of carbamylcholine induced negative chronotropic responses in the cells and tissues of the present invention.

Inducing of biological states and processes is preferably effected by treating the cells or tissues of the present invention with a treatment such as a treatment with a drug, a treatment with a physiological ion, and an electrical treatment. Alternately, inducing of biological states and processes may be effected by subjecting the cells and tissues to biomechanical stress treatment or to treatment with a growth or differentiation factor. Biomechanical stress treatment can be used, for example, to model various aspects of high blood pressure.

Preferably, the drug is selected from the group consisting of 1-heptanol, isoproterenol, carbamylcholine, forskolin, IBMX, atropine, tetrodotoxin, and diltiazem hydrochloride. As described hereinabove and in the following Examples section, these drugs can be used to induce selected biological states and processes, such as positive and negative chronotropic responses, and slowing of propagative electrical activity conduction in the cells and tissues of the present invention.

Preferably, the physiological ion is selected from the group consisting of a potassium ion, a sodium ion, and a calcium ion. Treatment with a physiological ion can be used, for example, to induce slowing of electrical activity conduction in the cells and tissues of the present invention. As is described and shown in the Examples section which follows, elevation of extracellular potassium can be used to induce slow conduction of propagative electrical activity, a clinically relevant cardiac abnormality, in the cells and tissues of the present invention.

Thus, according to yet an additional aspect of the present invention, there is provided a method of qualifying the effect of a treatment on a biological state or a biological process of cardiac cells or cardiac tissue. Qualifying the effect of a treatment on a biological state or a biological process of the cells and tissues of the present invention, for example an abnormal biological state or process thereof, can be used to identify and optimize treatments capable of restoring the normal biological state or process, and hence can be used to identify and optimize treatments suitable for treating cardiac disorders. Furthermore, qualifying the effect of a treatment on a biological state or a biological process of cardiac cells or tissues can be used to assess the toxicity of such a treatment on such a biological state or process. Since the method can be used to characterize the effect of a treatment on cardiomyogenesis, according to one embodiment the method can be used to assess the embryotoxicity of a treatment, in particular a treatment with a compound. Thus, failure to generate a characteristic associated with a cardiac phenotype, preferably cardiac specific mechanical contraction, in the cells and tissues of the present invention in response to treatment with a compound can be used to qualify the embryotoxicity, such as the cardiac specific or systemic embryotoxicity, of such a compound.

The method of qualifying the effect of a treatment on a biological state or a biological process of cardiac cells or tissue is effected by generating the cells or tissues of the present invention, as described hereinabove and in the Examples section below, subjecting the cells or tissues of the present invention to the treatment, and monitoring the biological state or process in such cells and tissues.

When qualifying the effect of a treatment in cells and tissues of the present invention co-cultured with primary cardiac cells or primary cardiac tissue, the treatment can be effected prior to or following such co-culturing or transplanting, allowing the determination of the effects of the treatment in these separate contexts.

Thus, this aspect of the present invention can be preferably utilized to determine the therapeutic and toxic effects of various treatments, such as drug treatments, and electrical treatments, on abnormal biological states and processes of cardiac cells and tissues, such as pathogenic biological states or processes of cardiac cells or tissues. Hence the method of the present invention can be used to screen and/or test cardiac drugs.

This aspect of the present invention can be utilized to obtain gene expression profiles and changes thereof in cardiomyocytic cells and tissues subjected to a treatment. Thus, the method according to this aspect of the present invention can be used to determine, for example, gene expression pattern changes in response to a treatment.

According to yet this aspect of the present invention, qualifying the effect of a treatment can be effected by following the effect of the treatment on electric conduction and electric activity in the cells or tissues of the present invention.

Preferably, the treatment to which the cells or tissues of the present invention are subjected is an exposure to a compound or an electrical treatment. Preferably the compound is a drug, or a physiological ion. Alternately the compound can be a growth factor or differentiation factor, such as, for example, vascular endothelial growth factor (VEGF).

Since drugs used for treating various diseases including cardiac diseases can unexpectedly result in desired or undesired blockage of ionic channels in heart tissue, they may ultimately lead to modification of the myocardial electrophysiological substrate and to the development of lethal arrhythmias, a property termed proarrhythmia. Hence, it is highly desirable to have a method which is capable of predicting such side effects on cardiac tissue prior to administering the drug.

Thus, according to another embodiment of the present invention, the cardiomyocyte multielectrode array used by the present invention can also be used to screen drug effects on electrical conduction and/or activity in cardiac cells and/or tissues.

Exposure to a compound is preferably effected as previously described in detail hereinabove and in the Examples section which follows, for example as described for compounds inducing chronotropic responses or changes in propagative electrical activity conduction.

Exposure to an electrical treatment is preferably effected by applying an electrical source to the cells and tissues of the present invention, for example via an electrode of a multielectrode array, as shown in Example 3 of the Examples section, or via any of the numerous suitable methods described in the literature of the art.

Subjecting the cells and tissues to a biomechanical stress treatment may be effected via any of numerous techniques known in the art. For example, biomechanical stress may be applied to the cells and tissues of the present invention using glass micropipettes controlled, for example, by a micromanipulating device.

Preferably, monitoring biological states and processes in the cells and tissues of the present invention following treatment thereof is preferably effected as extensively detailed hereinabove and in the Examples section which follows.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4, 666, 828; 4, 683, 202; 4, 801, 531; 5, 192, 659 and 5, 272, 057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8^{th} Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Generation of Highly Differentiated, Highly Functional Cardiomyocytic Cells Via In-Vitro Culture of Human Embryonic Stem Cells

Despite decades of intensive research, heart failure remains the primary cause of death and, to date, there do not exist satisfactory methods of generating cardiomyocytic cells which could be used therapeutically or as a satisfactory in-vitro model of cardiac development and function. Thus, in order to fulfill this critical need, the present inventors have generated for the first time in-vitro functional embryonic stem cell-derived cardiomyocytic cells, as follows.

### Materials and Methods:

Generation of human embryonic stem cell-derived cardiomyocytic cells: Ten different human embryonic stem cell lines including line H9 or its single cell clonal derivative H9.2 (Amit, M. et al., 2000. Dev. Biol. 227: 271) were individually grown on a mitotically inactivated (mitomycin C) mouse embryonic fibroblast feeder cell layer in culture medium, as previously described (Thomson, J. A. et al., 1998. Science 282: 1145). The culture medium consisted of 80% knockout DMEM (no-pyruvate, high-glucose formulation; Life Technologies, Inc., Rockville, Md., USA.) supplemented with 20% FBS (HyClone, Logan, Utah, USA.), 1 mM L-glutamine, 0.1 mM mercaptoethanol, and 1% nonessential amino acid stock (all from GIBCO-BRL).

To induce differentiation, embryonic stem cells were dispersed into small clumps (3-20 cells) using collagenase IV (Life Technologies, Inc., 1 mg/ml for 20 minutes). Dispersion of embryonic stem cells using trypsin, as is routinely used to generate cardiomyocytic cells from murine embryonic stem cells, was found to be highly damaging, leading, for example, to a high incidence of abnormal karyotypes.

The cells were then transferred to plastic Petri dishes (Miniplast, Ein Shemer, Israel), at a cell density of about 5×10⁶ cells in a 58 mm dish, where they were cultured under nonadherent conditions for 7-10 days. During this stage the cells aggregated to form embryoid bodies, which were then plated on 0.1% gelatin-coated culture dishes and observed microscopically for the appearance of spontaneous contractions. The different stages in generation of embryonic stem cell-derived cardiomyocytic cells are summarized in FIG. 1a.

In order to assess the efficacy of the cardiomyocyte differentiation system, a total of 1,884 embryoid bodies were plated on gelatin-coated dishes and monitored microscopically daily for the presence of contractions for up to 30 days postplating. The percentage of embryoid bodies displaying contracting areas, as well as the distribution of the timing of onset of spontaneous beating were evaluated. In a preliminary study, it was noted that varying embryonic stem cell density input in the suspension phase, while modifying the number of embryoid bodies produced, did not affect the percentage of beating embryoid bodies. Similarly, screening of two different lots of serum also did not modify the cardiomyocytic cell yield significantly. In addition, the effect of DMSO, a known stimulant of differentiation into cardiomyocytic lineage was assessed by adding dimethyl sulfoxide (Sigma Chemical Co., St. Louis, Mo., USA.) at a concentration of 0.75% (vol/vol) to the culture medium during the 10 days of growth in suspension. The percentage of contracting embryoid bodies as well as the timing of onset of spontaneous contractions were examined microscopically.

Immunostaining: Contracting areas were mechanically dissected using a pulled-glass micropipette and enzymatically dispersed using trypsin-EDTA (0.5% trypsin, 0.53 mM EDTA; Life Technologies, Inc.) for 15 minutes at 37° C. The dispersed cells were plated on laminin-coated glass coverslips, incubated for 48 h, fixed in 4% paraformaldehyde with sucrose, and permeated using 0.5% Triton X-100 (Sigma). The fixed and permeated cells were blocked with 10% BSA and incubated with primary antibody overnight at 4° C. Labeling with primary antibody was performed using mAbs specific for myosin cardiac heavy chain α/β at a dilution of 1:50, mAbs specific for cardiac muscle troponin I at a dilution of 1:5000, mAbs specific for desmin at a dilution of 1:100, and polyclonal antibody specific for atrial natriuretic peptide at a dilution of 1:250 (all from CHEMICON International, Inc., Temecula, Calif., USA). Staining of sarcomeric α-actinin and nebulin was performed using anti sarcomeric α-actinin mAbs at a dilution of 1:800 and anti nebulin mAbs at a dilution of 1:200 (both from Sigma), respectively. Following three washes with PBS, primary antibody stained cells were incubated with: FITC-conjugated donkey anti mouse IgG absorbed against human tissue for cardiac myosin heavy chain, desmin, and troponin I staining; or rhodamine-conjugated anti rabbit IgG for α-actinin, atrial natriuretic peptide, and nebulin staining (both from CHEMICON International) at a dilution of 1:100 for 1 h at room temperature. Immunostained preparations were examined by fluorescence microscopy. Dispersed cells isolated from noncontracting embryoid bodies served as controls.

RT-PCR: Total RNA from undifferentiated embryonic stem cells and contracting embryoid bodies was extracted using TRI reagent kit (Sigma), according to the manufacturer's instructions. Complementary DNA (cDNA) was synthesized from 1 μg total RNA using SuperScript II reverse transcriptase (Life Technologies, Inc.) and used as template for PCR amplifications with primers selective for human cardiac genes. The PCR primers and the reaction conditions used are listed in Table 1. The PCR products were size fractionated by 2% agarose gel electrophoresis.

Transmission electron microscopy: For transmission electron microscopy, the spontaneously contracting areas were mechanically dissected, fixed in 3% glutaraldehyde in 0.1 M cacodylate buffer, pH 7.4, at 4° C. for 24 h, postfixed in 1% OsO₄ in the same buffer for 1 h, dehydrated in graded ethanols, and embedded in Epon 812. Thin sections (250-300 nm) were used for ultrastructural evaluation using a JEOL 100 SX transmission electron microscope (Peabody, Mass., USA.) operating at 80 kV.

TABLE 1
PCR primers employed

| | | Reaction | | | |
|---|---|---|---|---|---|
| | | conditions | | | Product |
| Gene | | # | T_{A} | MgCl₂ | size |
| product | Primer pairs | cycles | (° C.)** | (mM) | (bp) |
| hANP | GAACCAGAGGGGAGAGACAGAG | 35 | 61 | 1 | 406 |
| | (SEQ ID NO: 1) | | | | |
| | CCCTCAGCTTGCTTTTTAGGAG | | | | |
| | (SEQ ID NO: 2) | | | | |
| MHC-2A | ACAGAGTTTATTGAGGTGCCCC | 35 | 61 | 1 | 381 |
| | (SEQ ID NO: 3) | | | | |
| | AAGGTGAAGTGTCCCAGAGG | | | | |
| | (SEQ ID NO: 4) | | | | |
| MHC-2V | TATTGGAACATGGCCTCTGGAT | 35 | 61 | 1 | 382 |
| | (SEQ ID NO: 5) | | | | |
| | GGTGCTGAAGGCTGATTACGTT | | | | |
| | (SEQ ID NO: 6) | | | | |
| GATA-4 | AGACATCGCACTGACTGAGAAC | 30 | 60 | 1 | 475 |
| | (SEQ ID NO: 7) | | | | |
| | GACGGGTCACTATCTGTGCAAC | | | | |
| | (SEQ ID NO: 8) | | | | |
| α-MHC | GTCATTGCTGAAACCGAGAATG | 40 | 61 | 2 | 413 |
| | (SEQ ID NO: 9) | | | | |
| | GCAAAGTACTGGATGACACGCT | | | | |
| | (SEQ ID NO: 10) | | | | |
| Oct-4 | GAGAACAATGAGAACCTTCAGGAGA | 35 | 55 | 1.5 | 219 |
| | (SEQ ID NO: 11) | | | | |
| | TTCTGGCGCCGGTTACAGAACCA^{§} | | | | |
| | (SEQ ID NO: 12) | | | | |
| Nkx2.5 | CTTCAAGCCAGAGGCCTACG | 35 | 55 | 1.5 | 233 |
| | (SEQ ID NO: 13) | | | | |
| | CCGCCTCTGTCTTCTTCAGC^{†} | | | | |
| | (SEQ ID NO: 14) | | | | |
| cTnT | GGCAGCGGAAGAGGATGCTGAA | 35 | 60 | 1.5 | 150 |
| | (SEQ ID NO: 15) | | | | |
| | GAGGCACCAAGTTGGGCATGAACGA^{‡} | | | | |
| | (SEQ ID NO: 16) | | | | |
| cTnI | CCCTGCACCAGCCCCAATCAGA | 35 | 60 | 1.5 | 250 |
| | (SEQ ID NO: 17) | | | | |
| | CGAAGCCCAGCCCGGTCAACT^{‡} | | | | |
| | (SEQ ID NO: 18) | | | | |
| GAPDH | AGCCACATCGCTCAGACACC | 25 | 61 | 1.5 | 302 |
| | (SEQ ID NO: 19) | | | | |
| | GTACTCAGCGGCCAGCATCG^{¶} | | | | |
| | (SEQ ID NO: 20) | | | | |

hANP - human atrial natriuretic peptide,
MHC - myosin heavy chain,
Oct-4 - octamer-binding protein-4;
^{§}(Abdel-Rahman, B. et al., 1995. Hum. Reprod. 10: 2787-2792);
^{†}(Shiojima et al., 1996. Circ. Res. 79: 920-929);
^{‡}(Ricchiuti V. and Apple S., 1999. Clin. Chem. 45: 2129-2135);
^{¶}(Itskovitz-Eldor et al., 2000. Mol. Med. 6: 88-95);
TA = annealing temperature

Multielectrode array (MEA) extracellular electrophysiology mapping and pharmacological studies: Intact contracting areas within the embryoid bodies were mechanically dissected using a pulled-glass micropipette and plated on gelatin-coated multielectrode arrays (Multi Channel Systems MCS GmbH, Reutlingen, Germany; Igelmund, P. et al., 1999. Pflugers Arch. 437: 669). The microelectrode array consists of 60 titanium nitride electrodes with gold contacts 30 μm in diameter with an interelectrode distance of 100-200 μm. The contracting areas were plated on top of the microelectrode arrays and cells were grown to confluence over the electrodes. In some embryoid bodies with relatively small contracting areas, the contracting area did not cover all electrodes, and hence recordings were performed from fewer than 60 electrodes. Extracellular signals were recorded simultaneously from all 60 electrodes at 25 kHz and band-pass filtered from 1 to 3000 Hz. Recordings were performed in culture medium at 37° C. A pH of 7.4 was maintained using perfusion with an atmosphere containing 5% CO₂ and 95% air. Chronotropic responses were assessed by extracellular recordings for 10 minutes prior to and following replacement of the culture medium with culture media containing 1 μM isoproterenol, 1 μM carbamylcholine, 10 μM 3-isobutyl-1-methylxanthine (IBMX), or 1 μM forskolin (all from Sigma).

Measurement of intracellular calcium ([Ca²⁺]ᵢ) transients: The embryoid bodies were loaded with fura-2-AM (Molecular Probes, Eugene, Oreg., USA.) for 25 minutes at room temperature (24-25° C.) at a final concentration of 5 μM, in a 1:1 mixture of Tyrode's solution. The dye loaded embryoid bodies were then transferred to a nonfluorescent chamber mounted on the stage of an inverted microscope (Diaphot 300, Nikon, Tokyo, Japan). The chamber was perfused with Tyrode's solution at a rate of 1 ml/minute and the temperature in the chamber was maintained at 37° C. Fura-2 fluorescence was measured using a dual-wavelength system (Deltascan, Photon Technology International [PTI], New Jersey, USA) as previously described (Felzen, B. et al., 1998. Circ. Res. 82: 438). Briefly, two different wavelengths (340 and 380 nm) were used for excitation, and the emitted fluorescence (510 nm) was collected and detected by a photomultiplier tube (710 PMT, photomultiplier detection system; PTI). Raw data were stored for off-line analysis using the FeliX software (PTI) as 340 nm and 380 nm counts and as the ratio R=F₃₄₀/F₃₈₀. The Savitzky-Golay smoothing algorithm was used to reduce noise level.

Statistical analysis: Data are expressed as mean ±standard deviation. In order to assess possible chronotropic effects, the average spontaneous beating rate was compared prior to and following drug application using two-tailed paired Student's t test. P values less than 0.05 were considered significant.

### Experimental Results:

Human Embryonic Stem Cell-Derived Cardiomyocytic Cells form Spontaneously Contracting Areas:

Typical examples of embryonic stem cell colonies grown on top of the mouse embryonic fibroblast feeder layer, the formation of embryoid bodies during the suspension phase, and an embryoid body containing a contracting area following plating are shown in FIGS. 1b-d, respectively. Out of the ten different embryonic stem cell lines subjected to the culturing protocol described above in the Materials and Methods section, only embryoid bodies derived from line H9.2 displayed a significant incidence of cardiomyocytic differentiation. Thus, all results presented herein were generated using cell line H9.2.

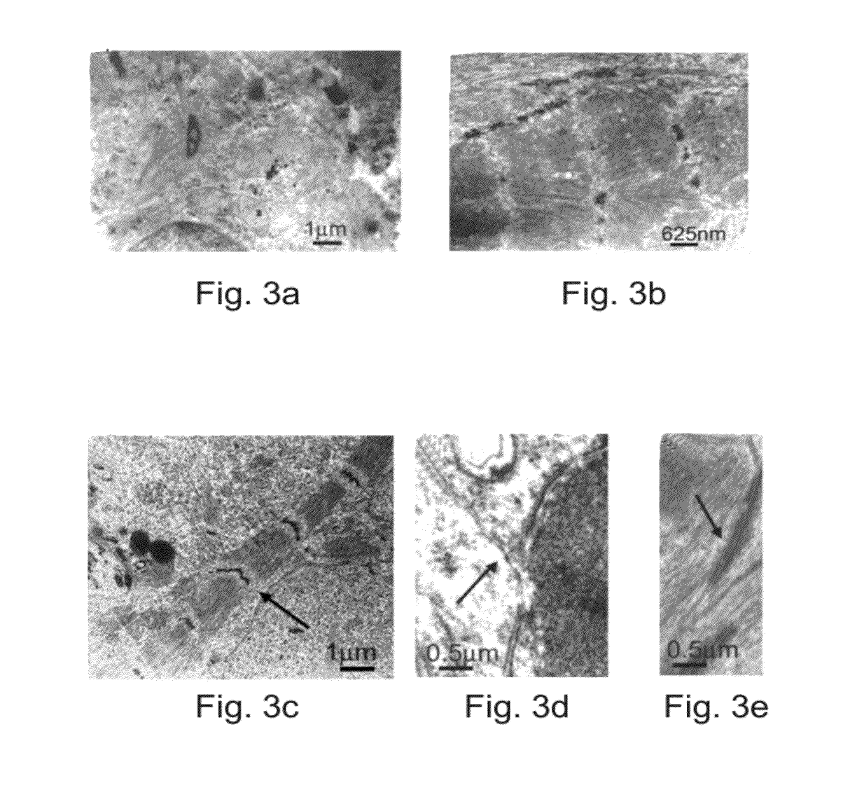

Embryoid bodies were found to develop rhythmically contracting areas between 4 and 22 days postplating, as shown in FIG. 2 which illustrates the cumulative percentage of embryoid bodies containing contracting areas as a function of days postplating. Such contracting areas appeared in 153 (8.1%) of the 1,884 embryoid bodies studied. In contrast, the incidence of spontaneous contraction observed in embryoid bodies derived the H9.2 parental line H9 was about 0.1%. The onset of contractions peaked between 6 and 11 days postplating, when contracting areas appeared in 4.9% of the embryoid bodies. The contracting areas usually appeared in the outgrowth of the embryoid bodies, displayed diameters ranging between 0.2 and 2 mm, and continued to beat vigorously for up to 60 days, the longest period studied. The presence of DMSO at a concentration of 0.75% increased the percentage of embryoid bodies demonstrating contracting areas to 10.1%.

Human Embryonic Stem Cell-Derived Cardiomyocytic Cells form Organized Sarcomeric Structures, Z-Bands, Intercalated Discs, Gap Junctions and Desmosomes:

Light microscopy revealed that the contracting areas were composed mainly of relatively small mononuclear cells, 10-30 μm in diameter, with round or rod-shaped morphology. Transmission electron microscopy of these cells revealed mononuclear cells displaying parallel arrays of myofibrillar bundles oriented in an irregular manner in some cells (FIG. 3a), while a more mature sarcomeric organization was apparent in others (FIGS. 3, b and c). The degree of myofibrillar organization varied within different areas of the same cell, among different cells in the same embryoid body, and among different embryoid bodies. Nevertheless, in general, a shift from an immature phenotype manifested by disorganized myofibrillar stacks in early stage embryoid bodies (FIG. 3a) to a more organized sarcomeric structure in later stage embryoid bodies (FIGS. 3b-c) was noted. In some foci, the formation of early and more developed Z bands could be observed (FIGS. 3b-c). The intercalated disc, another cellular structure that characteristically appears during in-vivo cardiomyocyte differentiation, was observed in many of the differentiating embryoid bodies. Intercalated discs composed of gap junctions and desmosomes were observed to connect adjacent cells (FIGS. 3d-e).

### Display of Fibrillar Bundle Striation and Expression of Cardiac Muscle Specific, but not Skeletal Muscle Specific Proteins, by Cultured Human Embryonic Stem Cell-Derived Cardiomyocytic Cells:

Using immunocytochemistry, the presence of cardiac-specific proteins and their spatial organization were studied in dispersed cells forming contracting embryoid bodies. FIG. 4a shows positive immunostaining of dispersed myocytes with anti cardiac α/β-myosin heavy chain mAbs. Varying degrees of myofibrillar organization were noted among the cells. The staining patterns ranged from cytoplasmic clamps in some cells to nonparallel bundles of elongated fibrillar structures in others. Some of these bundles displayed an early striated pattern (FIG. 4b). The contracting myocytes also stained positively with anti α-actinin mAbs (FIG. 4c), anti cTnI mAbs (FIG. 4d), and anti desmin mAbs (FIG. 4e), with different cells demonstrating varying degrees of sarcomeric organization. In addition, the speckled perinuclear staining by anti atrial natriuretic peptide (FIG. 4f) suggested the presence of cytoplasmic atrial natriuretic peptide granules. In contrast to the positive staining with cardiac-specific proteins, cells from the contracting areas did not demonstrate nebulin immunoreactivity, confirming the cardiomyocytic, rather than skeletal, nature of the cells (Begum, S. et al., 1998. Cell Tissue Res. 293: 305). In order to determine the percentage of cardiomyocytic cells in the contracting areas, the regions exhibiting spontaneous contracting activity were microdissected, enzymatically dispersed, and plated at low density to allow identification of individual cells by immunocytochemistry. Using anti cTnI mAbs, the percentage of positively stained cells was found to be 29.4%.

### Cultured Human Embryonic Stem Cell-Derived Cardiomyocytic Cells Exhibit Expression of Cardiac Specific Genes:

The expression of several cardiac-specific genes was assessed in the cultured human embryonic stem cell-derived cardiomyocytic cells and in undifferentiated human embryonic stem cells using RT-PCR. As shown in FIG. 5, myocytes from contracting embryoid bodies expressed the cardiac transcription factors GATA-4 and Nkx2.5 as well as the cardiac-specific genes cTnI, cardiac troponin T (cTnT), atrial myosin light chain (MLC-2A), ventricular myosin light chain (MLC-2V), and α-myosin heavy chain. GATA-4 and MLC-2A expression was also noted in the undifferentiated embryonic stem cells, but was markedly increased in the contracting embryoid bodies. Octamer-binding protein 4, a marker of undifferentiated cells, was expressed in the embryonic stem cells. This expression significantly declined in the contracting embryoid bodies. The housekeeping gene GAPDH served as an internal control.

### Display of Synchronous Contractions and Cardiac Specific Intracellular Calcium Transients by Cultured Human Embryonic Stem Cell-Derived Cardiomyocytic Cells:

Intracellular calcium transients ([Ca²⁺]ᵢ) were measured from spontaneously contracting embryoid bodies using fura-2 (n=7). FIG. 6a displays a typical recording with an initial rise in systolic [Ca²⁺]ᵢ and a slower decay. The time to peak systolic [Ca²⁺]ᵢ averaged 130±27 milliseconds, the time to half-peak relaxation was 143±94 milliseconds, and total transient length was 465±180 milliseconds. In all cases, the [Ca²⁺]ᵢ signals were synchronous with the contraction rate observed microscopically. FIG. 6b depicts a continuous recording.

### Human Embryonic Stem Cell-Derived Cardiomyocytic Cells Exhibit Cardiomyocyte Specific Extracellular Electrophysiology and Chronotropic Responses to Pharmacological Agents:

Extracellular recordings from the contracting areas displayed electrograms consisting of a sharp component with a peak-to-peak amplitude of 630±33 μV lasting 30±25 milliseconds, followed by a slow component of 347±120 milliseconds, representing the depolarization and repolarization processes, respectively (FIG. 6c). The average spontaneous beating rate was 94±33 beats/minute (n =8) and was stable (mean frequency standard deviation of 1.55 beats/minute) during a recording period of 20 minutes in all embryoid bodies studied.

Positive and negative chronotropic responses were observed following administration of the β-agonist isoproterenol and the muscarinic agonist carbamylcholine, respectively. Isoproterenol at 10⁻⁶ M significantly increased spontaneous contraction rate to 146±43% of its baseline value (n=8; P<0.01). Similarly, the direct adenylate cyclase activator forskolin and the phosphodiesterase inhibitor IBMX increased spontaneous contraction rate to 182±48% (n=6; P<0.01) and 152±77% (n=6; P<0.05) of its initial value. In contrast, the muscarinic agonist carbamylcholine at 10⁻⁶ M decreased the rate to 78±20% of its initial value (n=6; P<0.05). The latter effect was reversed by application of the muscarinic antagonist atropine. All the above responses were prompt, occurring within 90 seconds of drug application.

### Analysis:

The present invention provides a novel and reproducible system in which human embryonic stem cells differentiate into cardiomyocytic cells and tissue. These results demonstrate that the spontaneously contracting tissue within the developing embryoid bodies contain cardiomyocytic cells portraying structural and functional properties consistent with early stage cardiac tissue.

Several lines of evidence confirm the cardiomyocytic nature of these embryonic stem cell-derived cells. Ultrastructural analysis showed that these cells were mainly mononuclear and round or rod-shaped, contained different degrees of myofibrillar bundle organization, and exhibited nascent intercalated discs. These myofibrillar structures stained positively with anti human cardiac myosin heavy chain, anti α-actinin, anti desmin, and anti cTnI mAbs. The cells, however, did not exhibit immunoreactivity with anti nebulin mAbs, a specific skeletal muscle sarcomeric protein shown to be expressed early in skeletal myoblast differentiation (Begum, S. et al., 1998. Cell Tissue Res. 293:305).

These results are consistent with ultrastructural properties of early stage cardiomyocytes and with the developmental process of myofibrillar assembly. Previous studies demonstrated that during in-vivo cardiomyogenesis, myofibrils are initially distributed in sparse, irregular myofibrillar arrays, which gradually mature into parallel arrays of myofibrils and ultimately align into densely packed sarcomeres (Manasek, F. J., 1970. Am. J. Cardiol. 25:149, 22).

Embryonic stem cell-derived cardiomyocytic cells have previously been found to express a number of critical cardiomyocyte-specific genes, including those encoding the transcription factors GATA-4 and Nkx2.5 (Stainier DYR., 2001. Nat Rev Gen. 2:39). In addition to these, the genes for the cardiac specific proteins atrial natriuretic peptide, cTnI, cTnT, MLC-2A, MLC-2V, and α-myosin heavy chain were also found to be expressed in the embryonic stem cell-derived cardiomyocytic cells of the present invention. The presence of both MLC-2A and MLC-2V may suggest the presence of a number of cardiomyocytic cell types within the contracting areas.

The extracellular recordings, the [Ca²⁺]ᵢ transients, and the pharmacological studies clearly demonstrated that the contracting areas within the embryoid bodies displayed physiological properties consistent with cardiomyocytic tissue and significantly differed from noncardiac (skeletal or smooth) muscle. All components of normal cardiac excitation-contraction coupling were demonstrated within this tissue, namely electrical activation, increase in [Ca²⁺]ᵢ and the resulting contraction.

Little is known about calcium handling in the normal developing human heart. Intracellular calcium transients in adult human atrial tissue, studied using fura-2 (Brixius, K. et al., 1997. J Appl Physiol. 83:652), were similar to the ones recorded in the present study with respect to total duration of the [Ca²⁺]ᵢ transient and to time of half-peak calcium relaxation. However, the time to peak transient in the embryoid bodies (130±27 ms) was longer then the one obtained in human atrial myocytes (52.5±3.1 ms). This difference may have been the result of a number of causes. In the murine embryonic stem cell model, the contracting areas within embryoid bodies were shown to be a mixture of atrial, ventricular, and sinus nodal cells (Maltsev, V A. Et al., 1993. Mech Dev. 44:41). Since different cardiomyocyte types display different calcium characteristics (Kolossov E. et al., 1998. J Cell Biol. 143:2045), it is possible that the [Ca²⁺]ᵢ transients measured in whole beating embryoid bodies, described in the present invention, represent a superposition of a mixed population of myocytes with fast and slow cell characteristics. Alternatively, the lower rate in [Ca²⁺]ᵢ elevation may reflect the suboptimal efficiency and immaturity of the calcium machinery in early developing cardiac cells.

The extracellular recordings demonstrated a sharp and a slow component, consistent with the relatively long action potential duration characteristic of cardiomyocytes. The positive and negative chronotropic responses to isoproterenol and carbamylcholine demonstrated the presence of functional adrenergic and cholinergic receptors, respectively, in pacemaker cells. A major pathway of the β-adrenoreceptor-dependent chronotropic response is the activation of adenylate cyclase and the consequent rise in cytosolic cAMP and stimulation of protein kinase. The positive chronotropic effect exerted by forskolin, a direct activator of adenylate cyclase, and by IBMX, a phosphodiesterase inhibitor, suggests that this signaling pathway is already present early in human cardiomyocytic differentiation.

There are numerous differences between human and mouse developing cardiomyocytes. In the normal embryo, heart formation begins with the initiation of differentiation by myocardial and endocardial precursors and leads up to the formation of the cardiac valves. These events cover the first 12 days in the life of a mouse embryo and the first 35 days in the life of a human embryo (Stainier, DYR., 2001. Nat Rev Gen. 2:39). It is not surprising, therefore, that differentiation of human embryonic stem cells into cardiomyocytes proceeds at a slower rate than in mouse embryonic stem cells. In mouse models, embryonic stem cells are cultivated in hanging drops for 2 days, and further cultivated as embryoid bodies in suspension for 5 days. Spontaneously contracting areas appear 1 day postplating, and within 2-10 days 80-90% of embryoid bodies reveal pulsating areas (Wobus, A. M. et al., 1991. Differentiation 48:173). In the human embryonic stem cell differentiation system described herein, cells were grown in suspension for 10 days, and spontaneous contractions did not commence before day 4 postplating, with the median value being 11 days. Furthermore, only 8.1% of embryoid bodies revealed pulsating areas. These variations may represent differences between the species, differences between the cell lines, or some yet undetermined factor required in the in-vitro differentiation of human embryonic stem cells. Several factors that may be optimized in the future in order to increase cardiomyocytic cell yield include different serum content, length of suspension period, the use of growth factors, or the use of supporting stroma.

Morphologically, in-vitro differentiation of human and mouse embryonic stem cells appears to follow parallel pathways. The assembly of the Z-line from periodically aligned Z-bodies and the transition from disorganized myofibrils to the more organized sarcomeric pattern described here have also been noted in the mouse model (Hescheler J. et al., 1997. Cardiovasc Res. 36:149). In the two models, different degrees of myofibrillar assembly coexist within adjacent cardiomyocytic cells in the same embryoid body and within the same cell. Nevertheless, in the human model ultrastructural maturation proceeded much more slowly, seemed more heterogeneous, and did not reach the fully mature adult phenotype during the observation period.

### Conclusion:

These results therefore indicate that the present invention provides for the first time a method of generating unlimited numbers of human cardiomyocytic cells, including developing cardiomyocytic cells. The cardiomyocytic cells of the present invention display a broad range of cardiac tissue specific structural and functional characteristics, including: rhythmic synchronous contraction; formation of sarcomeres, Z-bands, intercalated discs, gap junctions, desmosomes and fibrillar bundles; expression of numerous cardiomyocyte specific, but not skeletal myocyte specific mRNAs and proteins; and cardiomyocyte specific electrophysiology, including cardiac specific chronotropic responses to pharmacological agents. Furthermore, the cardiomyocytic cells and tissues of the present invention display a sustained cardiomyocytic phenotype for at least 60 days in-vitro. Thus, the cardiomyocytic cells of the present invention are optimal, for example, for pharmacological and toxicological testing, functional genomics, early cardiomyogenesis, cell therapy, and tissue engineering. As such, the method of the present invention represents a dramatic and quantum improvement over all prior art methods of providing human cardiomyocytic cells.

### Example 2

### Generation of Highly Differentiated, Highly Functional Human Cardiomyocytic Tissue Via In-Vitro Culture of Human Embryonic Stem Cells

The ability to generate human cardiac tissue in-vitro would be of enormous benefit for therapy of heart diseases, for testing the therapeutic and toxic effects of pharmacological and electrical treatments of human cardiac tissue, and for modeling aspects of the biology of human cardiac tissue, such as cardiac tissue development and cardiac tissue physiology. However, no prior art methods of generating human cardiac tissue in-vitro exist. Thus, in order to fulfill these important needs the present inventors have generated, for the first time, highly differentiated, highly functional human cardiomyocytic tissues by in-vitro culture of human embryonic stem cells, as follows.

### Materials and Methods:

### Generation of Human Embryonic Stem Cell-Derived Cardiomyocytic Cells and Tissues:

Performed essentially as described in Example 1, hereinabove, for generation of human embryonic stem cell-derived cardiomyocytic cells. For generation of cardiomyocytic tissue, embryoid bodies were isolated at a stage of 24±9 days postplating (range, 12-54 days).

### Immunohistochemistry and Confocal Microscopy:

Embryoid bodies were fixed in 4% paraformaldehyde and then blocked with PBS containing 1% Triton X-100 and 2% normal goat serum for 15 minutes at room temperature. Immunostaining was performed using anti cardiac troponin I antibody (anti cTnI) at a dilution of 1:5000 and anti connexin-43 antibody (anti Cx43) at a dilution of 1:100 in the blocking buffer for 24 hours at 4° C. Preparations were then incubated with FITC-conjugated anti mouse and rhodamine-conjugated anti rabbit antibody at a dilution of 1:100 for 1 hour at room temperature (all antibodies from Chemicon, Temecula, Calif.). Confocal microscopy was performed using a Nikon Eclipse E600 microscope and Bio-Rad Radiance 2000 scanning system. These preparations were also used for morphometric studies in which the length, width, and length/width ratio of individual cultured human embryonic stem cell-derived cardiomyocytic cells were analyzed.

Quantitative analysis of the immunoreactive signal by confocal microscopy was performed as previously described (Saffitz J E. et al., 2000. Am J Physiol Heart Circ Physiol. 278:H1662; Thomas S P. et al., 2000. Circ Res. 87:467; Zhuang J. et al., 2000. Circ Res. 87:316) with some modifications. Briefly, 3-5 high-power fields in each embryoid body studied were examined by fluorescence microscopy at a magnification of ×60. Each test area, analyzed digitally, consisted of a matrix of 512×512 pixels. The immunoreactive signal for anti Cx43 was concentrated as discrete spots of high-intensity signal along the cell membrane, allowing automatic detection of gap junctions by use of an arbitrary threshold value. The proportion of total cell area occupied by the Cx43 immunoreactive signal was defined as the percentage of high-intensity signal pixels divided by the total number of pixels in the field. The total number and average size of gap junction, identified as two or more contiguous high-intensity pixels (≧0.32 μm²), were also analyzed from the immunostained specimens.

### Multielectrode Array Mapping Technique:

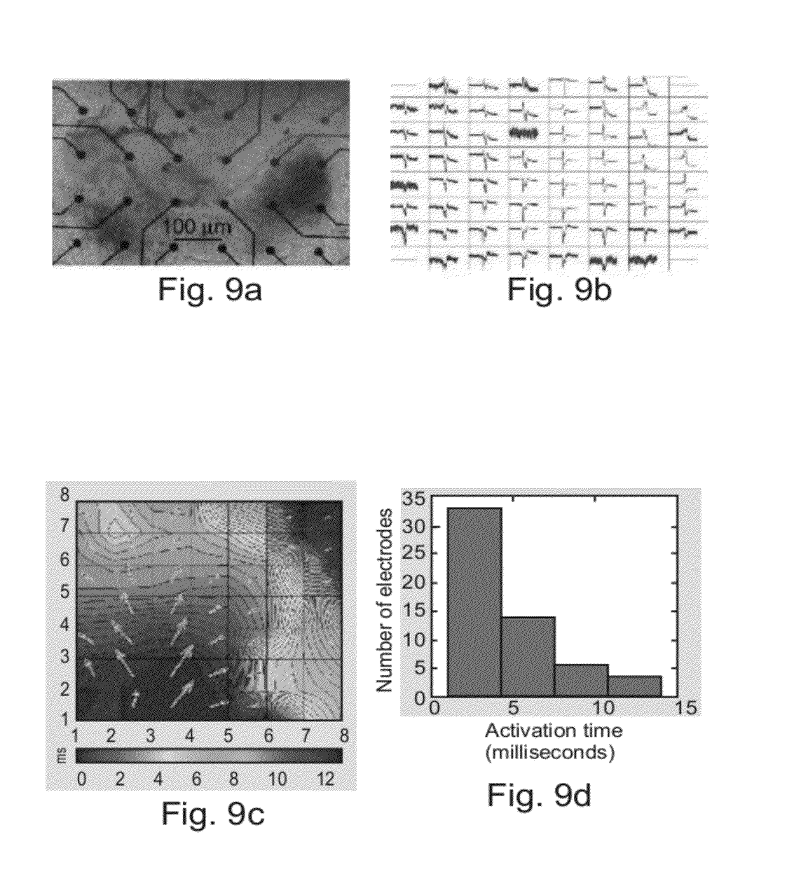

Extracellular electrophysiological recordings from the embryoid bodies were performed on a PC-based multielectrode array data acquisition system (Multi Channel Systems, Reutlingen, Germany). The microelectrode array consisted of a 50×50 mm glass substrate, in the center of which was an embedded 1.4×1.4 mm matrix of 60 titanium nitride-gold contact (30 μm) electrodes with an interelectrode distance of 100 or 200 μm (FIG. 7). This system allowed simultaneous recording of extracellular potentials from all electrodes for prolonged periods. Data were recorded at 10 kHz. During the recording sessions, the microelectrode array was constantly perfused with a gas mixture consisting of 5% CO₂ and 95% air. Temperature was kept at 37.0±0.1° C.

Local activation time (LAT) at each electrode was determined, as previously described (Spach M S. & Dolber P C. 1986. Circ Res. 58:356), by the timing of the maximal negative intrinsic deflection (dV/dtₘᵢₙ) of the electrogram recorded at each electrode. The measured LATs at all electrodes were then used for the generation of color-coded activation maps by interpolating the local activation time values between the electrodes using the standard two dimensional plotting function of MATLAB software (MATLAB 5.3.0, The MathWorks, Natick, Mass.).

Local conduction velocity vector fields were estimated as previously described (Bayly PV. et al., 1998. IEEE Trans Biomed Eng. 45:563), with some modifications. Briefly, each electrode was denoted by spatial and temporal (LAT) coordinates Zᵢ=[x, y, t]^{T}. Each sample point Zᵢ and its neighbors in each direction within a spatial window of Δxₘₐₓ, Δyₘₐₓ, and temporal window of Δtₘₐₓ were fitted using a least-square algorithm to a second-degree polynomial surface. The fitting spatial window size was chosen to be five times interelectrode distance, and the temporal window was five times the total activation time. The velocity vectors of each point on the wave front, v=[dx/dt, dy/dt]^{T}, were derived from the fitted surface. Calculations and plotting were performed using MATLAB.

### Pharmacological Analysis of Conduction:

Following baseline recording, 20 μl of stock solution of the test drug were added to the 2 ml of culture medium in the microelectrode array and stirred gently. The pharmacological agents used included tetrodotoxin (TTX; Alomone Labs, Jerusalem, Israel) at a final concentration of 10 μM, and diltiazem hydrochloride (1 μM, Sigma Chemical Co., St. Louis, Mo.). Extracellular recordings were performed for 30 seconds, at baseline and 5 minutes following drug application. 1-Heptanol (at 0.3, 0.6, and 1 mM; Sigma) was dissolved in the medium by vigorous shaking, and extracellular recordings were performed immediately prior to and 30 minutes following the application of the drug.

The effects of these drugs on conduction were evaluated by examining possible changes in the culture's global velocity (measured as the distance between earliest and latest activation divided by the total microelectrode array activation time), in the mean magnitude of the local velocity vector, and in the maximal absolute value of the first time derivative of the extracellular signal

In order to study the effects of elevated extracellular potassium concentration on conduction properties, a baseline recording was performed in a modified Tyrode' s solution (140 mM NaC1, 5.4 mM KC1, 10 mM glucose, 1 mM MgCl₂, 2 mM pyruvate, 2 mM CaCl₂, and 10 mM HEPES, titrated to pH 7.4 with NaOH). Extracellular recordings were then performed for 30 seconds, at baseline and 5 minutes after the solution was changed to three balanced high-concentration potassium solutions (10, 15, and 20 mM KC1).

### Statistical Analysis:

Values are mean±standard deviation. Changes in mean conduction velocity, local velocity vectors, and maximal absolute value of the first time derivative during elevation of the extracellular potassium were evaluated using Friedman's repeated measure ANOVA; comparisons with control were performed using Dunnett' s method. Changes during tetrodotoxin, diltiazem, and 1-heptanol treatments were assessed using Wilcoxon signed rank test. Differences with a P value of <0.05 were considered statistically significant.

### Experimental Results:

### Spontaneously Contracting Foci Express Cardiac Troponin I and Display Gap Junctions:

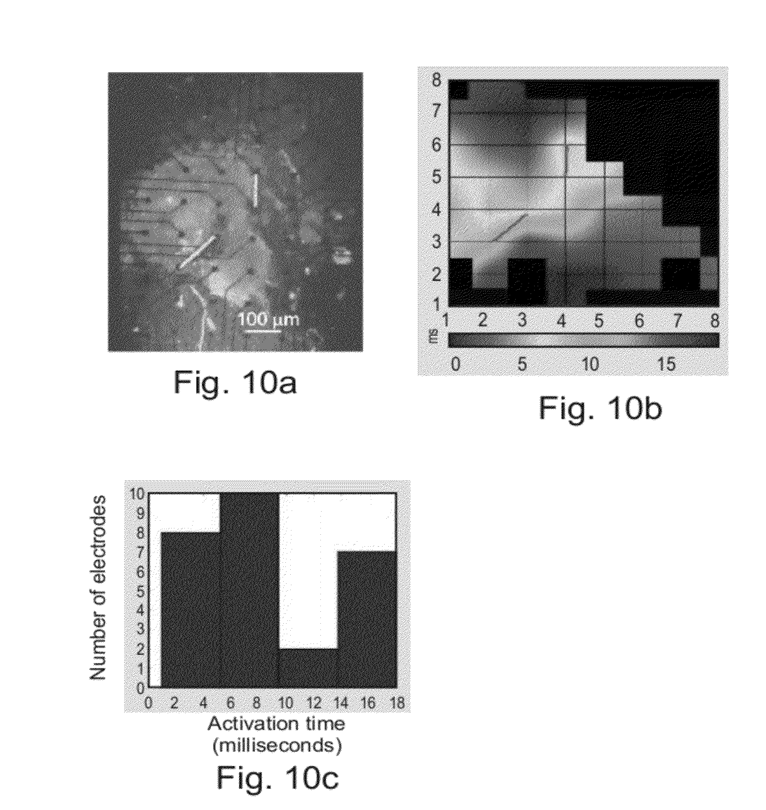

Spontaneously contracting human embryonic stem cell-derived cardiomyocytic foci were identified in the outgrowth of the embryoid bodies. The diameter of these contracting foci varied between 0.3 and 2 mm and their thickness was 0.04 to 0.1 mm (4 to 10 cell layers). The contracting areas were mechanically dissected and assessed morphologically. Immunostaining with anti-cardiac troponin I (cTnI) antibody (FIG. 8a) demonstrated the presence of an isotropic tissue consisting of early-stage cardiomyocytic cells. In some of the embryoid bodies, more than one contracting area could be noted, usually connected through a narrow strand of cardiomyocytic tissue. The cardiomyocytic cells within the contracting areas were relatively small; mononuclear; round, triangular, or rod-shaped; and arranged in various orientations. The cells displayed variable degrees of sarcomeric organization, ranging from a relatively homogenous cytoplasmic staining to a more developed early-striated pattern. Average cell length and width were 44.2±10.9 and 16.0±4.6 μm, respectively, with a mean length/width ratio of 2.9±0.9 (n=67). Noncardiomyocytes were also identified in the contracting areas (FIG. 8a) mainly at the periphery and accounted for 41±6% of all cells.

The presence of gap junctions between the cells was demonstrated by the positive punctate staining with anti connexin-43 (Cx43) and connexin-45 (Cx45) antibodies (FIGS. 8b-d). In contrast, connexin-40 (Cx40) was not identified in the cardiomyocytes and was seen, rarely, only in nonmyocytes. Based on Cx43 immunostaining, the average gap junction size was 0.58±0.08 μm². The number of gap junctions per 100 μm² tissue area was 0.45±0.18 and the proportion of tissue area occupied by high intensity immunoreactive signal was 0.27±0.13%. By using the Cx45 immunosignal, the same parameters were 0.57±0.12 μm², 0.88±0.33 and 0.48±0.13%, respectively. Double staining experiments demonstrated that most of the Cx43 and Cx45 immunosignals were colocalized to the exact same spots (same gap junctions).

Thus, these results demonstrate that ES cell-derived cardiomyocytes form relatively small gap junctions which are distributed homogeneously along the cell circumference with no preferential polar orientation. In addition, although Cx45 is almost absent in adult ventricular myocardium the positive immunostaining of Cx45 in this model is in line with the prior art data suggesting that Cx45 plays a major role in early cardiac embryonic development (Alcolea, S., et al., 1999, Circ. Res. 84: 1365-1379).

### High-Resolution Activation Mapping of Contractile Cardiomyocytic Foci:

Spontaneously contracting areas of cardiomyocytic syncytia were mechanically dissected and cultured on microelectrode array plates (FIG. 9a). This allowed long-term, high-resolution extracellular electrophysiological recordings from all 60 electrodes (FIG. 9b). The signals were characterized by a sharp component, a return to baseline, and a slow component resembling the T wave.

The detailed activation maps constructed using these recordings demonstrated the development of a functional syncytium with synchronized action potential propagation. FIG. 9c presents a typical activation map, with the propagation wave proceeding from the lower left corner to the top right corner of the microelectrode array and with a total activation time of 13 milliseconds. Impulse propagation can also be appreciated in the superimposed conduction velocity vectorial map.

Interestingly, both pacemaker position and conduction properties within each embryoid body were relatively reproducible during both short-term and long-term recordings. Thus, the average standard deviation of pacemaker position (identified by the site of earliest activation) was 103±100 μm during relatively short-term (3-hour) recordings and was also relatively stable (264±146 μm) during long-term recording (an average recording period of 9.5±5.4 days). Similarly, the average deviation in total activation time, global velocity (measured as the distance between earliest and latest activation divided by total activation time), and the mean magnitude of the local velocity vector were 8±3, 8±5, and 6±3%, respectively, during the short-term recordings and 18±13, 20±8, 21±14%, respectively, during long-term recordings.

In contrast with the relatively reproducible measurements within each embryoid body, the conduction properties observed in different embryoid bodies were more heterogeneous. In general, two conduction types were noted. In the first type (n=6), a single, relatively broad area of cardiomyocytic tissue was present, resulting in relatively fast conduction (FIG. 9c) throughout the measured area. This is also evident in the activation time histogram (FIG. 9d), which demonstrates a single cluster of continuous activation times from early to late LATs. Total activation time in these embryoid bodies averaged 10.3±3.8 milliseconds, and the average magnitude of the local conduction velocity vector was 14.2±9.5 cm/second.

The second type of conduction was observed in embryoid bodies, in which a relatively narrow strand of conducting tissue interconnected two or more contracting areas. A typical example is shown in the micrograph in FIG. 10a, where the cardiomyocytic tissue is identified by immunostaining with anti cTnI antibody. The corresponding activation map recorded from this embryoid body is shown in FIG. 10b. Note the presence of relatively fast activation within the two contracting areas (with very early or very late activation times), with significant time delay between them due to the narrow slow-conducting connecting strand. This can also be appreciated in the activation time histogram, which demonstrates the presence of very early or very late activated electrodes and paucity of electrodes with intermediate local activation time values (FIG. 10c). This type of conduction pattern resulted in significant longer activation times (30.6±18.9 milliseconds [n=6]) than in the first group (P<0.05) and in a lower magnitude of the average conduction velocity vector (4.4±2.9 cm/second). Nevertheless, fast conduction was still present in the broader parts of these embryoid bodies.

### Pharmacologically Induced Slowing of Conduction in Human Embryonic Stem Cell-Derived Cardiomyocytic Cells:

The suitability of the cardiomyocyte electrophysiology model of the present invention for the study of slow conduction was assessed. It is well established that the three main mechanisms involved in conduction slowing are reduced excitability, reduced cell-to-cell coupling, and abnormalities in tissue architecture. Hence, by using different pharmacological strategies, slowing of conduction via each of these mechanisms was demonstrated, as follows.

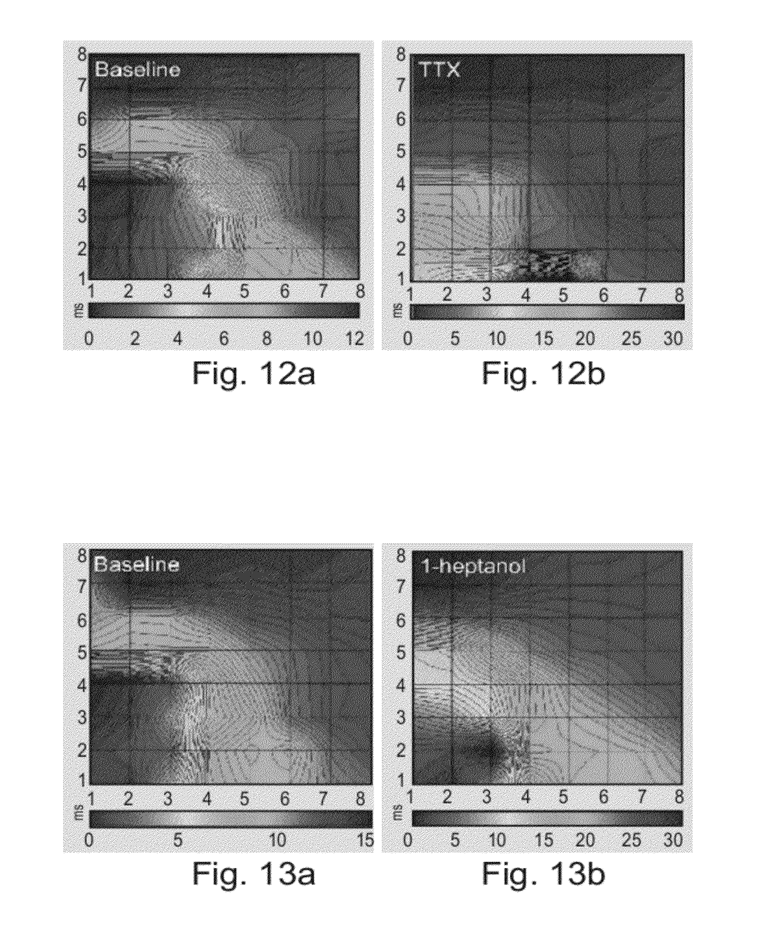

Elevation of the extracellular potassium concentration was found to result in slowing of conduction, as manifested by an increase in total activation times from a baseline value of 25 milliseconds (in 5.3 mM potassium; FIG. 11a) to values of 40, 70, and 120 milliseconds at extracellular potassium concentrations of 10, 15, and 20 mM, respectively (FIGS. 11b-d, respectively). Slowing of conduction during elevation of the extracellular potassium concentration was evident in all embryoid bodies studied (n=7; Table 2) by the significant decrease in global conduction velocity (P<0.01), in the mean magnitude of the local velocity vector (P<0.01), and in the maximal absolute value of the first time derivative of the extracellular signal (−dV/dtₘₐₓ, P<0.01).

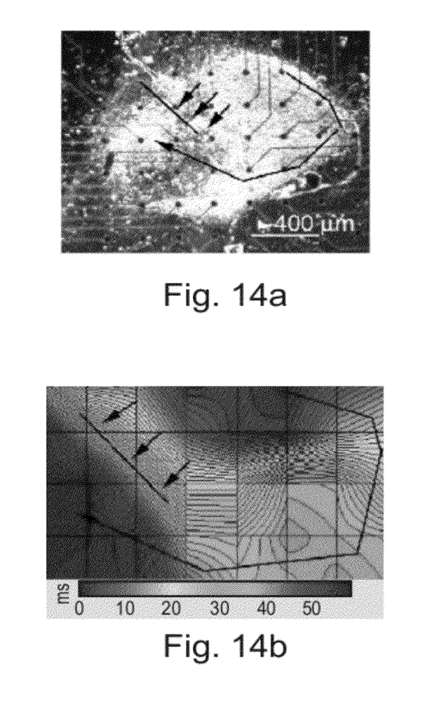

Activation maps of the same embryoid body, during baseline and following application of 10 μM of the fast sodium channel blocker tetrodotoxin (TTX) (FIGS. 12a-b, respectively) demonstrate slowing of conduction, as evidenced by the increase in the total microelectrode array activation time from a baseline value of 15 milliseconds to a value of 35 milliseconds. Blockade of the fast sodium channel resulted in slowing of conduction in all embryoid bodies, as manifested by the significant changes in all the electrophysiological parameters (n=6; Table 2) that were assessed. In contrast to these results, blockade of the L-type calcium channel by diltiazem (1 μM) did not result in significant changes in any of the parameters studied (n=6; Table 2).

TABLE 2
Pharmacologically induced changes in conduction properties

| | | | Maxi- |
|---|---|---|---|
| | Global | Mean CV | mal − dv/dt |
| Treatment | Velocity | Vector | (mV/sec) |
| Potassium—5.4 mM | 2.1 ± 1.0 | 4.1 ± 4.1 | 3.1 ± 6.4 |
| Potassium—10 mM | 1.6 ± 0.6^{†} | 3.5 ± 4.2 | 2.7 ± 5.5 |
| Potassium—15 mM | 1.0 ± 0.3^{†} | 2.0 ± 2.3^{†} | 1.1 ± 1.4 |
| Potassium—20 mM | 1.0 ± 0.4^{†} | 1.5 ± 1^{†} | 1.1 ± 1.2^{†} |
| TTX—Baseline | 3.2 ± 2.7 | 5.1 ± 6 | 2.1 ± 2 |
| TTX—10 μM | 1.8 ± 0.9* | 3.2 ± 3^{†} | 1.9 ± 1.9^{†} |
| Diltiazem—Baseline | 2.8 ± 1.9 | 6.0 ± 8.2 | 2.3 ± 2.6 |
| Diltiazem—1 μM | 2.8 ± 2.0 | 5.7 ± 4.2 | 2.5 ± 4.2 |
| Heptanol—Baseline | 2.1 ± 0.9 | 4.3 ± 5.3 | 2.8 ± 3.1 |
| Heptanol—0.3 mM | 1.7 ± 0.6* | 3.0 ± 5.7^{†} | 2.0 ± 2.3^{†} |
| Heptanol—0.6 mM | 1.2 ± 0.9 | 3.0 ± 3.8^{†} | 2.1 ± 3.0^{†} |
| Heptanol—1 mM | No conduction | No conduction | No conduction |

*p < 0.05 or
^{†}p < 0.01 when compared to baseline recordings.

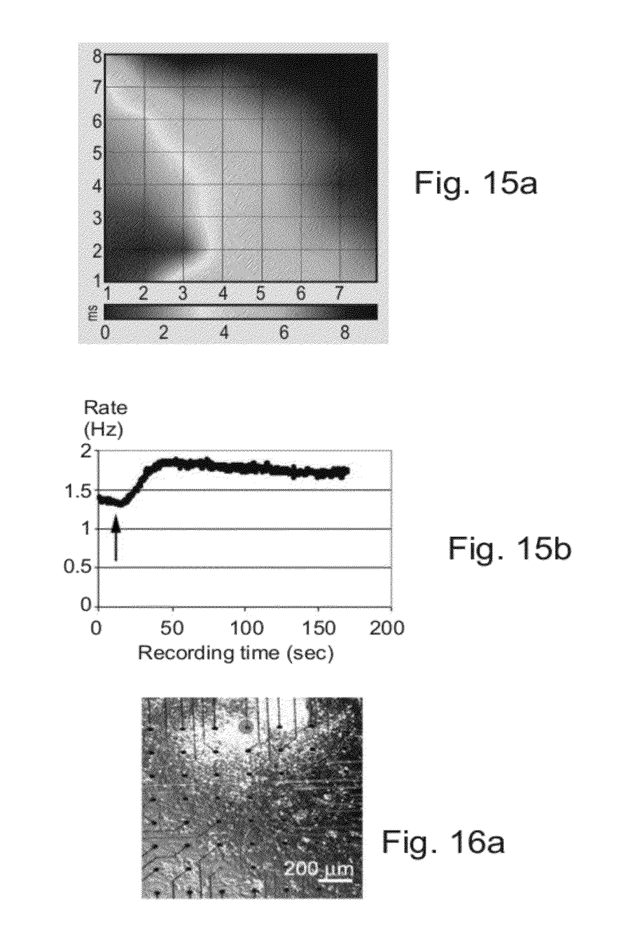

Reduction of cell-to-cell coupling also resulted in slowing of conduction. Activation maps of the same embryoid body, during baseline and following application of 0.3 mM of 1-heptanol (FIGS. 13a-b, respectively) demonstrate an increase in total activation time from a baseline value of 15 milliseconds to a value of 32 milliseconds. The slowing of conduction by 0.3 mM of 1-heptanol was noted in all embryoid bodies studied (n=7; Table 2). Higher doses of 1-heptanol (0.6 and 1 mM) resulted in complete cessation of beating (n=8). In four of the embryoid bodies, conduction was still maintained at 0.6 mM, but was significantly slower.

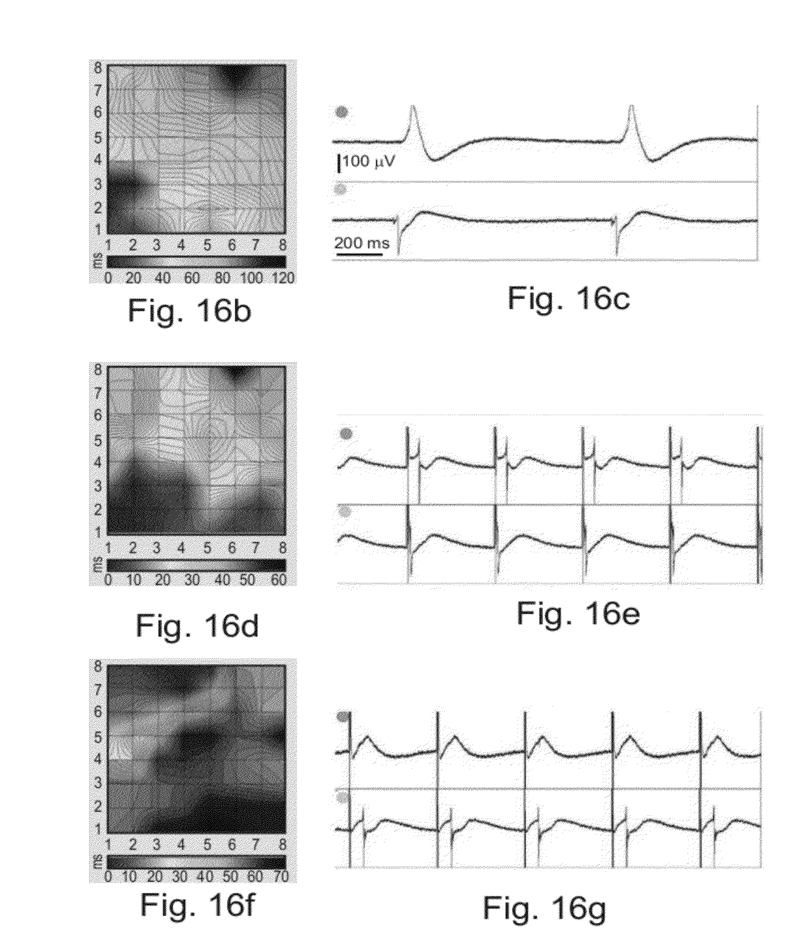

In a few preparations, the presence of strands of noncontracting tissue interspersed within the beating tissue were noted. These structural inhomogeneities resulted in the appearance of relatively slow conduction. One example can be seen in FIGS. 14a-b in which the presence of a noncontracting tissue resulted in alteration of the propagation pathway. The resulting activation wavefront curvature was associated with slowing of conduction, as can be appreciated by the long activation time (70 milliseconds) in this example.

### Analysis:

### Structural Properties:

The human embryonic stem cell-derived cardiomyocyte syncytia of the present invention were composed of one or a number of contracting foci connected by narrow strands, resulting in an inhomogeneous three dimensional aggregate of cardiomyocytic cells. While different from the two dimensional-patterned monolayers of neonatal rat and mouse cardiomyocytes used in other models, the pattern observed in this study was similar to the one described in the murine embryonic stem cell-derived cardiomyocytic model (Igelmund et al., 1999. Flugers Arch. 437:669). The cardiomyocytic cells within the embryoid bodies varied in size and shape and were composed of relatively small, early-stage cardiomyocytic cells oriented in various directions. Again, this pattern was different from the above-mentioned patterned monolayers and from the adult human heart.

This pattern of gap junction distribution was similar to the one observed by (Peters N S. et al., 1994. Circulation 90:713) in human fetal and neonatal tissue, in which gap junctions and fascia adherens junctions in infantile ventricular tissue were distributed as small punctate spots of Cx43 and N-cadherin immunoreactive signals along the entire surface of the cells. The pattern of gap junction number, size, and distribution is an important determinant of conduction and has been shown to change in several physiological and pathological conditions (Saffitz J E. et al., 2000. Am J Physiol Heart Circ Physiol. 278:H1662; Kanno S. and Saffitz J E. 2001. Cardiovasc Pathol. 10:169; Peters N S. et al., 1997. Circulation 95:988; Peters N S. and Wit A L. 1998. Circulation 97:1746; Saffitz J E. et al., 2000. Circ Res. 86:723; Saffitz J E. et al., 1999. Cardiovasc Res. 42:309; Patel P M. et al., 2001. J Cardiovasc Electrophysiol. 12:570).

### Conduction Properties:

The gross structural morphology of the cardiomyocytic tissue within the embryoid bodies had a significant effect on conduction properties.

Significantly slower conduction velocities were observed in embryoid bodies in which narrow strands of conducting tissue interconnected two or more contracting areas relative to embryoid bodies containing a single, relatively broad area of cardiomyocytic tissue. A similar structural pattern was also described in a murine embryonic stem cell model (Igelmund P. et al., 1999. Pflugers Arch. 437:669). Long-term electrophysiological recordings of the contracting embryoid bodies in that model demonstrated complex activity patterns due to intermittent blocks of action potential propagation in these narrow strands. Significantly, in the human model, these narrow bands, while significantly slowing conduction, did not cause similar conduction blocks.

The conduction velocity values observed in the current study, even in the relatively broad areas (14.2±9.5 cm/second), were lower then the values reported in neonatal rat and mouse monolayers, which averaged 20-50 cm/second (Thomas S P. et al., 2000. Circ Res. 87:467; Zhuang J. et al., 2000. Circ Res. 87:316). The slower conduction observed here might have been the result of the relatively small cellular dimensions, the isotropic nature of the tissue, the heterogeneous distribution and lower density of the cells within the cellular network, and the lower gap junction size and density. It is important to note, however, that the measured velocity represents the two dimensional projection of the velocity vector within the three dimensional embryoid body.

Although significant differences in the conduction properties were noted between different embryoid bodies, most probably due to variability in the structural properties of the tissues, the electrophysiological parameters measured within each embryoid body (pacemaker location and conduction velocity parameters) were reproducible during both short-term and long-term recordings. This is important since it allows assessing the effects of different pharmacological interventions as well as acute and long-term effects of remodeling processes using each embryoid body as its own control.

### Modeling of Slow Conduction:

The effects of reduction in excitability, decrement in cell-to-cell coupling, and alterations in tissue architecture, three key ingredients in the development of slow conduction (Rohr S. et al., 1999. Trends Cardiovasc Med. 9:173; Rohr S. et al., 1998. Circ Res. 83:781), an important factor associated with onset of cardiac arrhythmia were modeled using the method of the present invention. The results obtained were genetically consistent with the established role of these factors in the development of slow conduction. Analysis of the effects of decreased excitability on conduction using the fast sodium channel blocker TTX and elevation of extracellular potassium concentration demonstrated that depressed excitability resulted in a significant reduction in conduction velocity. The ability of TTX to decrease conduction and the absence of a similar effect by application of a selective calcium blocker demonstrated the presence and important role of the fast sodium current in impulse conduction within the embryonic stem cell-derived cardiomyocytic tissue during normal conduction. Nevertheless, persistence of conduction following TTX application (known to cause almost complete block of sodium channel at this concentration) and also during the presence of very high extracellular potassium concentration indicated that conduction, although significant slowed, could presumably be maintained under such conditions mainly via calcium channels. Similar results were obtained in a rat monolayer model (Rohr S. et al., 1998. Circ Res. 83:781).

Reduction of cell-to-cell coupling induced by 1-heptanol administration also resulted in significant slowing of conduction, while higher doses of this agent resulted in conduction block. Significantly, the conduction velocity values obtained prior to the appearance of conduction block were not lower than those observed following reduction of excitability. This is different from previous observations in the neonatal rat model (Rohr S. et al., 1998. Circ Res. 83:781), which showed a higher safety factor for the uncoupling agents. This difference might have resulted from the limitation of the uncoupler used here, 1-heptanol, which may also affect several membrane currents. In addition to the above-mentioned mechanisms, it was also noted that alterations in tissue architecture also generated significant slowing of conduction. This was evidenced by the development of slow conduction in embryoid bodies containing narrow strands of conducting tissue, most probably due to sink-source mismatches. A similar slowing of conduction was noted in embryoid bodies in which the presence of nonconducting tissue resulted in significant curving of the activation wavefront.

### Conclusion:

These results therefore indicate that the present invention provides, for the first time, a means of generating an excitable human cardiac syncytium with synchronized action potential propagation. Such a syncytium can be used to investigate the structural and functional properties of cardiac tissue as well as to assess physiological and pathophysiological phenomena such as altered and slow conduction. The present invention furthermore provides, for the first time, a long-term human cardiomyocyte culture which can be used to study phenomena, such as cardiac electro-mechanical activity, on a long-term scale in cardiomyocytic tissue in-vitro. As such, the present invention is unique and far superior to all prior art methods of generating human cardiac tissue in-vitro suitable for treatment of cardiac diseases, testing the effect of compounds on cardiac cells and tissues, and for elucidation of cardiac tissue development and function.

### Example 3

### Pacing of the Ventricle in a Complete Heart Block Model using Cardiomyocytic Cells Generated by Cultured Human Embryonic Stem Cells

Adult heart tissue has very poor regenerative capacity and therefore any significant cell loss or dysfunction of such tissue is essentially irreversible. Many types of cardiac diseases, such as myocardial infarction, are associated with loss of cardiac tissue function, and may lead to the development of progressive heart failure. Tissue loss or dysfunction, occurring at critical sites in the conduction system of the heart, may also lead to inefficient rhythm initiation or impulse conduction. Consequentially, these processes may result in abnormally low heart rate (bradyarrhythmias) requiring the implantation of a permanent pacemaker. Cell therapy has been suggested as a novel therapy for restoration of the myocardial electromechanical functions. However, this approach is hampered by the lack of a human source for cardiac tissue, and by the absence of evidence for functional integration between host and donor tissue. The present inventors have therefore demonstrated the feasibility of using human embryonic stem cells to generate human cardiac tissue capable of restoring impaired cardiac function in-vivo, as follows.

### Materials and Methods:

### Generation of Human Embryonic Stem Cell-Derived Cardiomyocytic Cells and Tissues:

Performed as described above in Example 2 of the Examples section.

### Multielectrode Array (MEA) Extracellular Electrophysiology Mapping:

Performed as described above in Example 2 of the Examples section.

### Optical-Mechanical Detection:

Mechanical contractions were detected via microscope (Axiovert 135, Zeiss, Oberkochen, Germany) using a photodiode (UV100BG, EG&G, Vaudreuil, Canada). A 100-Watt Phillips projection lamp was used as a light source. Scattered light from the cultures was magnified with a 40× objective (Zeiss 440865). The emission field was limited to a contracting zone by placement of an adjustable rectangular diaphragm (Nikon Instruments Inc., Melville, N.Y.). The photodiode was positioned on top of a Microflex PFX (Nikon). The ocular finder was used for framing and focusing the desired area of the specimen. Signals were filtered using a bidirectional Butterworth fourth order low-pass digital filter to obtain zero phase distortion with a cut-off frequency of 3 kHz.

### Generation of Primary Cardiomyocyte-Human Embryonic Stem Cell-Derived Cardiomyocytic Hybrid Tissue In-Vitro:

Primary monolayer cultures of neonatal rat (Sprague Dawley) ventricular myocytes were prepared as previously described (Fast V G. and Kleber A G., 1993. Circ Res. 73:914). Briefly, following excision, the ventricles were minced and treated with RDB (IIBR, Ness-Ziona, Israel). Dispersed cells were then cultured on gelatin coated (0.1%) microelectrode array plates or on glass cover slips at a density of 1.5×10⁶ cells/ml. Spontaneously contracting areas within human embryonic stem cell-derived embryoid bodies were mechanically dissected and grafted to the neonatal rat cultures once these displayed well synchronous activities (3-5 days postplating).

Pacing studies in co-cultures were performed by electrically stimulating the human or rat tissues with one of the electrodes of the multielectrode array.

### Immunohistochemistry:

Immunostaining of human cells and gap junctions in rat-human co-cultures was performed using anti human HLA class I (Dako, Copenhagen, Denmark) and anti connexin-43 (Cx43; Chemicon Int. Inc., Temecula, Calif.) as primary antibodies, respectively, and FITC-conjugated anti rabbit IgG and Cy-3-conjugated anti mouse IgG (Chemicon) as secondary antibodies. Analysis was performed via confocal microscopy (Nikon Eclipse E600, Nikon Europe BV, Badhoevedorp, The Netherlands) using a Bio-Rad Radiance 2000 scanning system (Bio-Rad Laboratories, Hercules, Calif., USA). For identification of human cells in paraffin sections in transplantation studies (described hereinbelow), human embryoid bodies were labeled with the vital fluorescent lipophilic tracer CM-DiI (Molecular Probes, Inc., Eugene, Oreg., USA) prior to grafting, and human cardiomyocytic cells were detected by primary anti cardiac troponin I (cTnI) and Cy-2 conjugated anti mouse IgG antibody.

### Lucifer Yellow Dye Transfer:

In order to detect the presence of gap junctions between primary ventricular cardiomyocytes and cultured human embryonic stem cell-derived cardiomyocytic cells, rat cells growing in monolayer were dyed with lucifer yellow (ICN Biomedicals, Costa-Mesa, Calif.) prior to addition of human cells via scrape-loading with a pulled glass-pipette following addition of lucifer yellow to a concentration of 0.025%. The hybrid cultures were examined via confocal microscopy 24 hours following addition of the cultured human embryonic stem cell-derived cardiomyocytic cells. Lucifer yellow is a low-molecular weight dye that can pass through gap junctions.

Transplantation of Cultured Human Embryonic Stem Cell-Derived Cardiomyocytic Cells into Swine Heart Following Atrioventricular Block:

Seven domestic pigs (20-30 kg) were used in these studies, one of which died immediately following the initial procedure. The experimental protocol was approved by the Animal Study Committee of the Technion Faculty of Medicine. Anesthesia was induced with ketamine (10 mg/kg) and diazepam (1 mg/kg) intravenously and was maintained following intubation and mechanical ventilation with isofluorane 1%. Vascular access was obtained using cutdown of the jugular veins and carotid arteries.

A 7F locatable electrophysiological mapping catheter (Navistar, Boosense-Webster) was introduced under sterile conditions into the heart and was used to generate three dimensional electroanatomical maps of the heart using the Carto mapping technique. The catheter was then navigated to the His position (identified by the presence of clear His potential recording) and radiofrequency current was applied to generate complete atrioventricular block. A specially designed VVI pacemaker (ELA, France) was then implanted subcutaneously and its electrode was positioned at the RV apex to allow ventricular pacing in cases of decrease of ventricular rate to less than 50 beats/minute.

The chest was then opened under sterile conditions by left thoracotomy through the fourth intercostal space, the pericardium was removed and cultured human embryonic stem cell-derived cardiomyocytic cells (20-40 contracting embryoid bodies, each embryoid body contains approximately 20,000 cells) or medium were injected into two different sites in the myocardium at the lateral and anterior walls respectively. A suture was used to mark the exact locations where injections were made. The human embryonic stem cell-derived tissue was labeled prior to transplantation with the vital fluorescent lipophilic tracer CM-DiI (Molecular Probes), according to the manufacturer's instruction, to allow identification of the cells during the pathological examination.

Following the procedure, animals were treated with daily injections of cyclosporin A (15 mg/kg) and methylprednisolone (2 mg/kg) to prevent immune rejection. Body-surface ECG recordings were performed daily to characterize the rate and configuration of the escape rhythm.

One to three weeks posttransplant the animals were re-anesthetized, intubated and mechanically ventilated and detailed three dimensional electroanatomical maps of the (LV) were generated using the Carto three dimensional mapping technique. A catheter was navigated to the site of earliest activity identified on the map, and radiofrequency ablation was performed at a nearby position to allow pathological correlation with the maps.

Hematoxylin and eosin (H&E) staining was employed during pathological examination to visualize human cells engrafted within recipient cardiac tissue.

### Experimental Results:

Functional integration of Cultured Human Embryonic Stem Cell-Derived Cardiomyocytic Cells with Primary Ventricular Cardiomyocytes In-Vitro:

Activation maps of spontaneously contracting areas of cultured human embryonic stem cell-derived cardiomyocytic cells plated onto microelectrode array plates demonstrated the development of an excitable medium with a stable pacemaker. FIG. 15a presents a typical activation map, with the propagation wave proceeding from the lower left corner (earliest activation—red) to the top right corner (latest activation—blue), and with a total activation time of 13 milliseconds. The contraction of these embryoid bodies displayed positive chronotropic responses in the presence of 10 μM isoproterenol (FIG. 15b). Such cells were used to generate spontaneously and synchronously contracting hybrid primary ventricular-human embryonic stem cell-derived cardiomyocyte cultures on microelectrode array plates (FIG. 16a). Significantly, synchronous contraction within the hybrid cultures was detected microscopically as soon as 24 hours postgrafting. Clearly identified synchronous contraction was observed in all 22 hybrid cultures studied. A typical microelectrode array activation map generated during spontaneous rhythmic contraction is shown in FIG. 16b. Electrical activation initiated, in this case, within the rat tissue and then propagated to the rest of the hybrid culture, activating also the human tissue. Electrograms recorded simultaneously from the human and rat cardiomyocytic tissues (FIG. 16c) demonstrated tight temporal coupling. This tight electrophysiological coupling was observed continuously for up to 21 days, the longest period studied. Long-term action-potential propagation between the two tissue types was also observed in pacing studies, in which either the rat (FIGS. 16d-e) or the human tissue (FIGS. 16f-g) was stimulated with an electrode.

In order to assess the presence of electromechanical coupling between the tissues, the contractions in the cultured human embryonic stem cell-derived cardiomyocytic cells, detected by a photodiode, were correlated with the electrical activity. As can be seen in FIG. 16h the mechanical contractions detected in the human embryonic stem cell-derived cardiomyocytic cells were synchronous with the electrical activity in both the human and rat tissues.

### Pharmacological Studies:

The degree of electrical coupling between the two tissue types was also assessed during long term recordings and during adrenergic stimulation and partial gap junction uncoupling. The cycle-lengths of the electrical activity in the ES cell-derived cardiomyocytes and in the rat tissue were measured and a histogram depicting the ratio between these two cycle-lengths during long-term recordings is shown in FIG. 16i. The narrow peak at a ratio of 1 represents equal cycle-lengths and demonstrates that electrical activity in the EB was time-locked to that of the rat tissue. Isoproterenol (10 μM) caused a significant increase in the spontaneous beating rate (from a base line value of 1.6±0.6 to 1.9±0.8 Hz, p<0.05) but electrical coupling between the two cell types was not hindered (FIG. 16j). Similarly, mild gap junction uncoupling using 1-Heptanol (0.5 mM) did not alter this tight coupling in the majority (5 of the 8) of the co-cultures studied (FIG. 16l) while in 3 co-cultures occasional episodes of 2:1 conduction block were noted (FIG. 16k).

### Abundant Gap Junction Formation Between Cultured Human Embryonic Stem Cell-Derived Cardiomyocytic Cells and Primary Ventricular Cardiomyocytes:

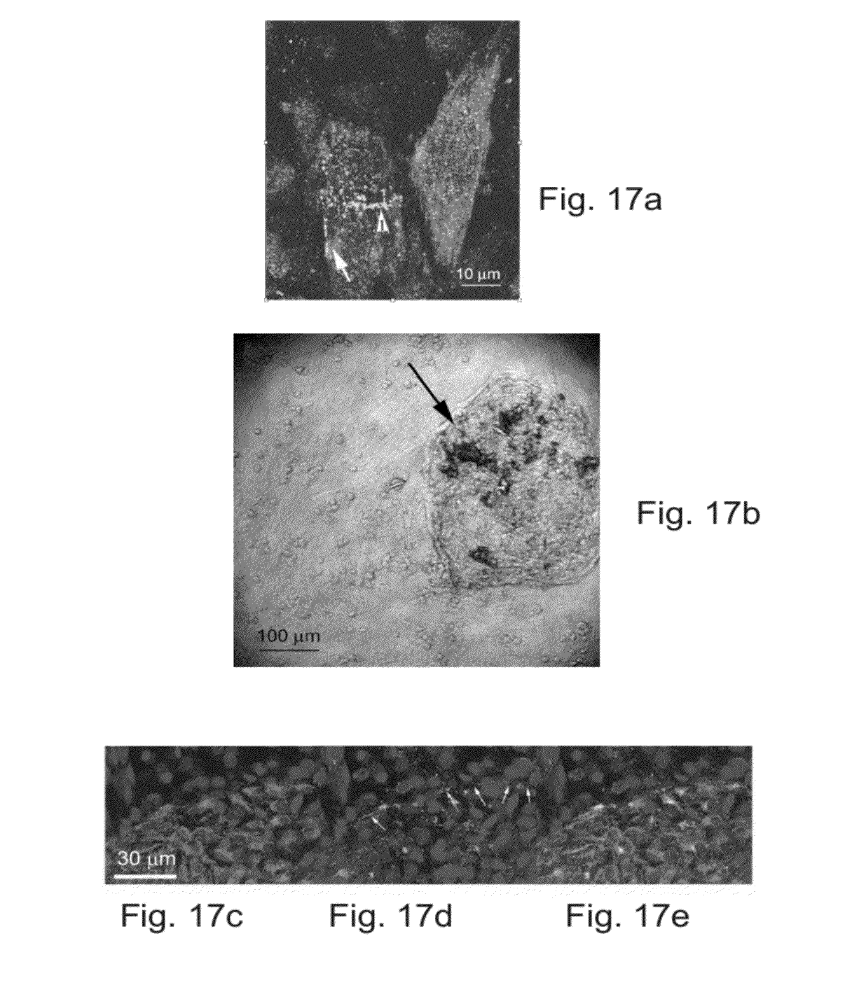

For electromechanical coupling to occur between primary cardiomyocytes and cultured human embryonic stem cell-derived cardiomyocytic cells, gap junctions must develop at the interface between these tissues. In order to detect such gap junctions, hybrid tissues were analyzed via immunofluorescent confocal microscopy for the presence of connexin-43, the major gap junction protein. The human embryonic stem cell-derived tissue was identified by labeling the cells with anti human HLA class I antibody. The results demonstrate abundant expression of connexin-43 in the rat cardiomyocytes, in the cultured human embryonic stem cell-derived cardiomyocytic cells and at the junction between the two tissues (FIG. 17a).

The presence of functional gap junctions was also demonstrated by the ability of lucifer yellow, which was scrape-loaded into the primary ventricular cardiomyocytes prior to grafting of human cells, to pass between the two cell types (FIG. 17b).

In addition, using a confocal microscope and double-staining with anti-human mitochondrial antibodies (FIG. 17c, red) and anti connexin-43 antibodies (FIG. 17d, green) the presence of connexin-43 was demonstrated at the interphase between the two tissue types (FIG. 17e).

### Restoration of Impaired Cardiac Function In-Vivo Following Transplantation of Cultured Human Embryonic Stem Cell-Derived Cardiomyocytic Cells:

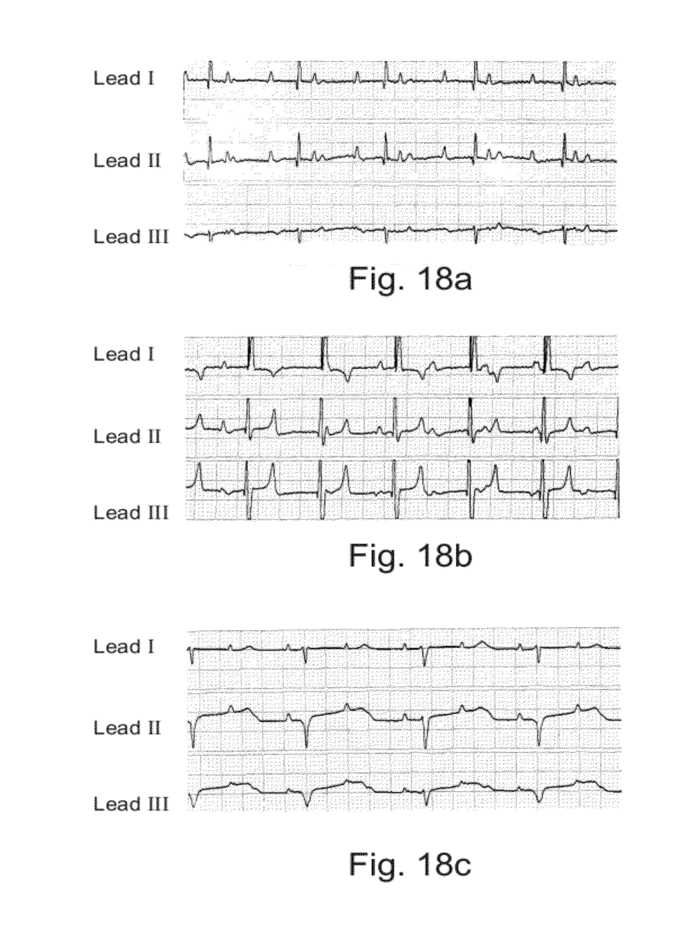

To demonstrate functional integration of cultured human embryonic stem cell-derived cardiomyocytic cells in heart tissue in-vivo, a swine model of complete atrioventricular block was used. A typical ECG recording following the creation of atrioventricular block is shown in FIG. 18a demonstrating complete atrioventricular dissociation with a typical nodal escape rhythm. In the initial days following creation of the atrioventricular block the nodal escape rhythm was occasionally very slow. In these cases episodes of overriding ventricular pacing originating from the VVI pacemaker occurred (FIG. 18b).

Following creation of atrioventricular block, spontaneously contracting human embryoid bodies were injected into the posterolateral LV wall using an epicardial approach, and the location of the injection was marked with a suture. A control injection of medium in the anterior wall in each animal was also performed and its location was marked by a different suture.

Several days following cell transplantation, episodes of a ventricular rhythm different from that of nodal or paced rhythms were observed (FIG. 18c). The morphology of the QRS of this new rhythm (negative axis in leads I, II, II) was completely different than that of nodal or paced rhythms, indicating its origin from a completely different focus. The average heart rate of this new rhythm was 68±4 beats/minute and was very similar to the swine's escape nodal rhythm (65±2 beats/minute) explaining the competition between the two rhythms. Significantly, the new rhythm was sensitive to catecholamines with the heart rate increasing to 105±10 beats/minute following administration of isoproterenol (20 μM).

In order to determine whether this new rhythm originates from the pacemaking action of the transplanted cells, the animals were subjected to an additional electrophysiological mapping procedure one to three weeks (average: 12 days) following human embryonic stem cell-derived cardiomyocyte transplantation. FIGS. 19a-b show the anteroposterior (AP) view of two maps obtained during the two different rhythms. In FIG. 19a, note the presence of earliest activation (red area) along the superior septum representing the origin of the nodal rhythm as identified immediately following generation of the complete atrioventricular block. In FIGS. 19c-d one can note that the earliest activation shifted from this area to the posterolateral wall during mapping of the new ventricular rhythm, two weeks following cell transplantation. This shift of the earliest activation site to the lateral wall can also be viewed on the left lateral view (FIG. 19d).

In order to determine whether this new rhythm actually originated from the transplanted cells the catheter was navigated to the earliest activation site and a focal ablation was performed by applying radiofrequency energy at a site about 2 cm away from the earliest activation point. FIGS. 19e-f show the pathological correlation in the same animal. In FIG. 19e, note that the distance between the ablation site (marked by the pink needle) and the injection site (blue suture) was 2.1 cm. This excellent spatial correlation was noted in all animals studied (n=8) with the average distance between the sites of earliest activation and ablation in the maps (19±5 mm) highly correlating (r²=0.93) with the average distance between the ablation and injection sites (20±5 mm) in pathology.

To verify the reproducibility of the location of the source of the new ectopic activity, the mapping procedure was repeated at two different days in some of the animals. As can be seen in FIGS. 19g-h, the maps, generated in the same animal, were highly reproducible with the earliest activation site located at the posterolateral region in both maps (arrows). An RF point ablation was created during each of these mapping procedures on opposite sides of the earliest site of activity. Note in FIG. 19i the excellent correlation in pathology with the site of cell injection (marked by a blue suture) located exactly between the two ablation sites (marked by the green needles).

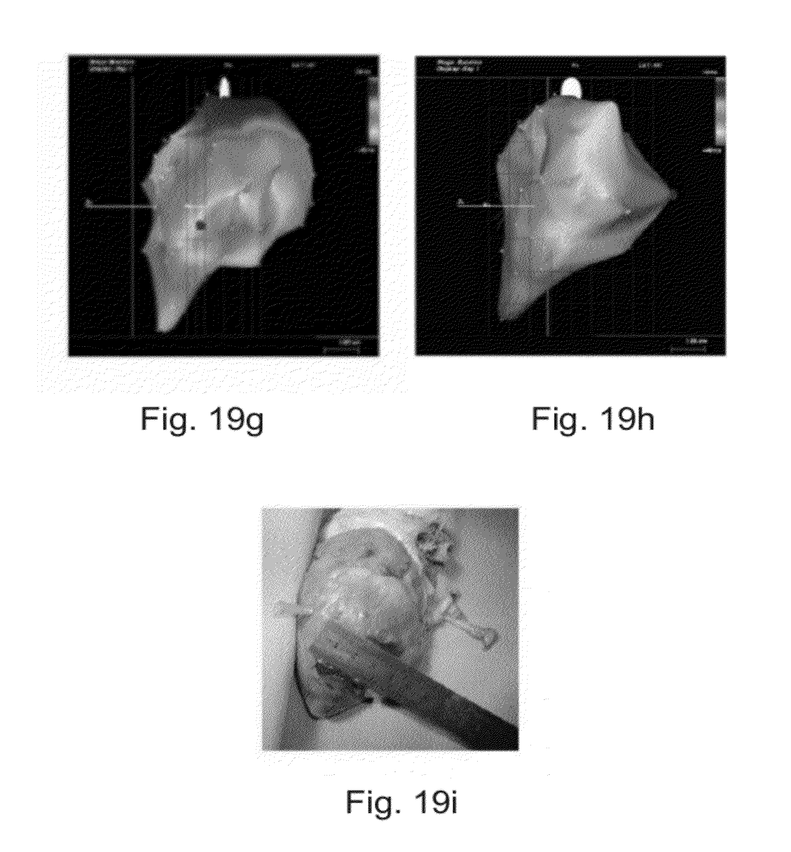

After harvesting the hearts, the presence of the grafted cells at the site of earliest electrical activation was further validated. Histological sections derived from the area of the previous electrical activation demonstrated the presence of the transplanted cells within the host myocardium (FIG. 20a). The transplanted cells, labeled before injection with the vital fluorescence tracer, CM-Dil, were further identified using fluorescent microscopy (FIG. 20b). The human phenotype of the transplanted cells was verified using immunostaining with anti-human mitochondrial antibodies (FIG. 20c). Finally, connexin-43 immunostaining confirmed the presence of gap junctions between the transplanted and host cells (FIG. 20d). In contrast to the above results, sections taken from distant myocardial areas failed to detect any of the transplanted cells.

### Conclusion:

These results demonstrate that the method of the present invention can be used to reproducibly derive cardiomyocytic cells from human embryonic stem cells having the capacity to form a cardiomyocytic syncytium displaying long term electromechanical activity, and that this tissue is able to form highly functional electromechanical connections with preexisting primary cardiac tissue whether in-vitro or in-vivo. Critically, when grafted in-vivo into heart tissue in a complete heart block model, the cultured human embryonic stem cell-derived cardiomyocytic cells of the present invention created functional connections and electrically activated the impaired host tissue. The method of the present invention is therefore unique and far superior to all prior art methods of providing embryonic stem cell derived human cardiac tissue suitable for transplantation and concomitant restitution of impaired cardiac function in-vivo.

### Example 4

### Cardiomyocytic Cells Generated by Culturing Human Embryonic Stem Cells have the Capacity to Proliferate

The extremely low capacity of adult cardiomyocytes to proliferate has to date represented an obstacle to approaches attempting to utilize in-vitro culture adult cardiomyocytes to generate cells and tissues suitable for repairing cardiac tissue. Thus, methods of generating cardiomyocytic cells and tissues capable of significant proliferation would be of great biomedical benefit. In order to fulfill this important need, the present inventors have uncovered that culturing of human embryonic stem cells according to the method of the present invention can be used to generate cardiomyocytic cells and tissues capable of significant proliferation, as follows.

### Materials and Methods:

### Generation of Human Embryonic Stem Cell-Derived Cardiomyocytic Cells and Tissues:

Performed essentially as described in Example 2, hereinabove.

### Cell Cycle Activity:

Whole EBs were fixed in 4% paraformaldehyde and blocked for 60 minutes with PBS containing 3% normal goat serum. Immunohistochemistry was performed using mouse anti-cTnI antibody (Chemicon, International, Inc. Temecula, Calif., USA) at a dilution of 1:5,000 and rabbit polyclonal anti-human Ki-67 antibody (Santa Cruz, Biotechnology, Inc., Santa Cruz, Calif., USA) at a dilution of 1:100 overnight at 4° C. Preparations were then incubated with Cy-2 conjugated anti-rabbit IgG and Cy-3-conjugated anti-mouse IgG (both from Chemicon) at a dilution of 1:100 for 1 hour at room temperature. Cell nuclei were counterstained with the DNA dye To-Pro-3 (Molecular Probes, Eugene, Oreg., USA). Confocal microscopy was performed using a Nikon Eclipse E600 microscope and Bio-Rad Radiance 2000 scanning system (Bio-Rad, Laboratories, Hercules, Calif., USA). The cardiomyocyte labeling index was then determined as the percentage of cardiomyocyte nuclei that were positively stained for Ki-67.

### Experimental Results:

### Cell Cycle Activity Decreases During In-Vitro Cardiomyocyte Development:

The expression of Ki-67, a marker of cell cycle activity, was used to evaluate cycling cells during in-vitro development of ES cell-derived cardiomyocytes. As is shown in FIGS. 21a-d, while a high percentage of Ki-67 positively stained nuclei are seen in early-stage cardiomyocytes (FIG. 21a), almost no expression of Ki-67 is seen in late-stage cardiomyocytes (FIG. 21c).

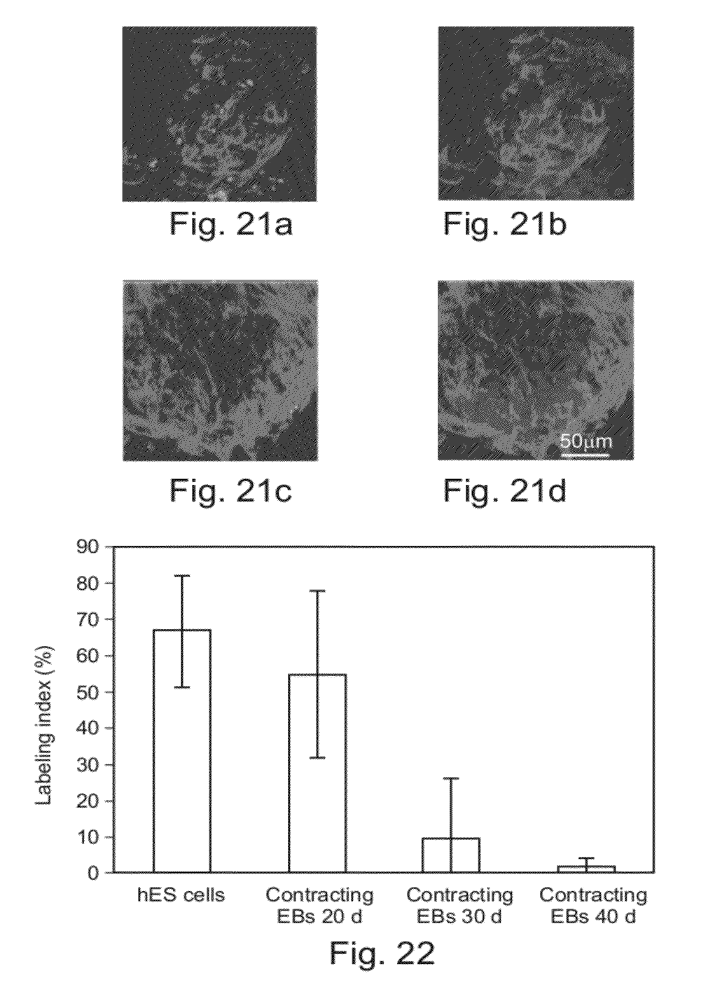

Analysis of the labeling index in ES cells and ES cell-derived cardiomyocytes revealed that undifferentiated stem were actively synthesizing DNA (labeling index >60%, FIG. 22). A similar high-labeling index was also noted in early-stage cardiomyocytes with 55±23% of the cells (FIG. 22). The labeling index gradually declined in intermediate-stage (30 days postplating) contracting EBs (10±16%) and was reduced to 2% in late-stage EBs (40 days postplating, p<0.05, FIG. 22).

### Conclusion:

Thus, the method of the present invention can be used to generate cultured human cardiomyocytic cells and tissues displaying significant capacity to proliferate for at least 30 days. The proliferative capacity of the cells and tissues of the present invention is unique relative to cells and tissues generated according to prior art methods, thus the cells and tissues of the present invention are optimal, for example, for therapeutic use in humans, notably for repair of cardiac tissue, for testing the effects of compounds on cardiac specific cell and tissue growth in-vitro, and for studying aspects of cardiomyogenesis, such as cardiomyogenesis specific gene expression profiles.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

What is claimed is:

1. A method of generating cells having a pacemaker activity and displaying at least one additional characteristic associated with a cardiac phenotype, the method comprising:
(a) culturing embryoid bodies on an adherent surface under culturing conditions that promote generation of cardiac cells having the pacemaker activity and displaying the at least one additional characteristic associate with a cardiac phenotype; and
(b) analyzing a pacemaker activity of said cardiac cells, thereby generating cells having a pacemaker activity and displaying at least one additional characteristic associated with a cardiac phenotype.

2. The method of claim 1, wherein said analyzing is performed by determining expression of functional adrenergic and cholinergic receptors.

3. The method of claim 1, wherein said analyzing comprises determining a β-adrenoreceptor-dependent chronotropic response.

4. The method of claim 3, wherein said response is a result of activation of adenylate cyclase and consequent rise in cytosolic cAMP and stimulation of protein kinase.

5. The method of claim 4, wherein said activation is by an agent selected from the group consisting of forskolin, a direct activator of adenylate cyclase and a phosphodiesterase inhibitor.

6. The method of claim 1, wherein said embryoid bodies are generated by;
(a) generating a cell population comprising aggregates of pluripotent stem cells; and
(b) subjecting said cell aggregates to culturing conditions suitable for generating embryoid bodies.

7. The method of claim 6, further comprising isolating said aggregates from said cell population prior to step (b).

8. The method of claim 6, further comprising isolating said embryoid bodies following step (b).

9. The method of claim 1, wherein said culturing conditions comprise culture medium supplemented with serum.

10. The method of claim 1, further comprising isolating cells which comprise a pacemaker activity.

11. The method of claim 6, wherein said human pluripotent stem cells are embryonic stem cells.

12. The method of claim 6, wherein said human pluripotent stem cells are H9.2 cells.

13. The method of claim 6, wherein step (a) is effected by partially dispersing a confluent cultured population of said human pluripotent stem cells.

14. The method of claim 13, wherein said partially dispersing is effected via a non-trypsin based method.

15. The method of claim 14, wherein said non-trypsin method comprises treatment with collagenase.

16. The method of claim 6, wherein said culturing in step (b) is effected for a time period selected from the range of 7 to 10 days.

17. The method of claim 1, wherein said culturing conditions in step (b) include culture medium supplemented with serum.

18. The method of claim 1, wherein said culturing is effected for at least as long as a time period selected from the range of 1-60 days.

19. The method of claim 1, wherein said culturing is effected in the presence of dimethyl sulfoxide.

20. The method of claim 1, wherein said adherent surface comprises a surface coated with gelatin.

21. The method of claim 1, wherein said at least one additional characteristic associated with a cardiac phenotype is selected from the group consisting of a cardiac specific structure, expression of a cardiac specific RNA, expression of a cardiac specific protein and cardiac specific changes in the intracellular concentration of a physiological ion.

22. A method of treating a cardiac disorder associated with cardiac arrhythmia in a subject in need thereof, comprising transplanting in the subject a therapeutically effective amount of cardiac cells having a pacemaker activity and at least one additional characteristic associate with a cardiac phenotype, said cardiac cells having been generated by culturing embryoid bodies on an adherent surface, thereby treating the cardiac disorder associated with cardiac arrhythmia.

23. The method of claim 22, wherein said cardiac arrhythmia is selected from the group consisting of sinus arrhythmia, premature beat, heart block, atrial fibrillation, atrial flutter, pulsus alternans and paroxysmal tachycardia.

24. The method of claim 22, wherein said transplanting comprises intramyocardial transplanting.

25. The method of claim 22, wherein said embryoid bodies are generated from embryonic stem cells.

26. The method of claim 22, wherein said at least one additional characteristic associated with a cardiac phenotype is selected from the group consisting of a cardiac specific structure, expression of a cardiac specific RNA, expression of a cardiac specific protein and cardiac specific changes in the intracellular concentration of a physiological ion.

27. The method of claim 22, further comprising isolating cells which comprise a pacemaker activity prior to said transplanting.
